(19) United States
(12) Patent Application Publication
(10) Pub. No.: US 2009/0042874 A1
(43) Pub. Date: Feb. 12, 2009

(54) ANTIDEPRESSANT HETEROARYL DERIVATIVES OF HETEROCYCLE-FUSED BENZODIOXANS

(75) Inventors: Amedeo A. Failli, Princeton Junction, NJ (US); Deborah Evrard, Hamilton Square, NJ (US); Yanfang Li, Lawrenceville, NJ (US); Nicole T. Hatzenbuhler, Bridgewater, NJ (US); Juliann Mogish, Morrisville, PA (US); Iwan S. Gunawan, Somerset, NJ (US); Antonina Nikitenko, Tarrytown, NY (US); Ping Zhou, Plainsboro, NJ (US)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 12/128,930

(22) Filed: May 29, 2008

Related U.S. Application Data
(60) Provisional application No. 60/940,765, filed on May 30, 2007.

(51) Int. Cl. C07D 491/22; C07D 413/14; A61K 31/536; A61K 31/436; A61P 25/30; A61P 25/18; A61P 25/00

(52) U.S. Cl. 514/230.5; 544/105; 514/291; 546/90

(57) ABSTRACT

The invention provides compounds of the Formula:

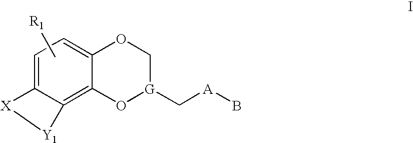

that are useful for the treatment of depression (including but not limited to major depressive disorder, childhood depression and dysthymia), anxiety, panic disorder, post-traumatic stress disorder, premenstrual dysphoric disorder (also known as pre-menstrual syndrome), attention deficit disorder (with and without hyperactivity), obsessive compulsive disorder, social anxiety disorder, generalized anxiety disorder, obesity, eating disorders such as anorexia nervosa, bulimia nervosa, vasomotor flushing, cocaine and alcohol addiction, sexual dysfunction and related illnesses.

This application claims benefit of priority to U.S. provisional patent application No. 60/940,765, filed on May 30, 2007, which is hereby incorporated by reference in its entirety.

### FIELD OF THE INVENTION

This invention relates to antidepressant heteroaryl derivatives of heterocycle-fused benzodioxans, to processes for preparing them, methods of using them and to pharmaceutical compositions containing them.

### BACKGROUND

Major depressive disorder affects more than 5% of the population, with a lifetime prevalence of 15-20%. Depression is the most frequently diagnosed psychiatric disorder and, according to the World Health Organization, is the fourth greatest public health problem. If left untreated, the effects of depression can be devastating, robbing people of the energy or motivation to perform everyday activities and, in some cases, leading to suicide. Symptoms of the disorder include feelings of sadness or emptiness, lack of interest or pleasure in nearly all activities, and feelings of worthlessness or inappropriate guilt. In addition to the personal costs of depression, the disorder also has been estimated to result in more than $40 billion in annual costs in the United States alone, due to premature death, lost productivity, and absenteeism.

Selective serotonin reuptake inhibitors (SSRIs) have had significant success in treating depression and related illnesses and have become among the most prescribed drugs. Some of the most widely known SSRIs are fluoxetine, sertraline, paroxetine, fluvoxamine and citalopram. Although they have a favorable side effect profile compared to tricyclic antidepressants (TCAs), they have their own particular set of side effects due to the non-selective stimulation of serotonergic sites. They typically have a slow onset of action, often taking several weeks to produce their full therapeutic effect. Furthermore, they have generally been found to be effective in less than two-thirds of patients.

SSRIs are believed to work by blocking the neuronal reuptake of serotonin, increasing the concentration of serotonin in the synaptic space, and thus increasing the activation of postsynaptic serotonin receptors. Although a single dose of a SSRI can inhibit the neuronal serotonin transporter, and thus would be expected to increase synaptic serotonin, clinical improvement has generally been observed only after long-term treatment. It has been suggested that the delay in onset of antidepressant action of the SSRIs is the result of an increase in serotonin levels in the vicinity of the serotonergic cell bodies. This excess serotonin is believed to activate somatodendritic autoreceptors, i.e., 5-HT_{1A} receptors, reduce cell firing activity and, in turn, decrease serotonin release in major forebrain areas. This negative feedback limits the increment of synaptic serotonin that can be induced by antidepressants acutely. Over time, the somatodendritic autoreceptors become desensitized, allowing the full effect of the SSRIs to be expressed in the forebrain. This time period has been found to correspond to the latency for the onset of antidepressant activity [Perez, V., et al., The Lancet, 1997, 349: 1594-1597].

Much information concerning the serotonin 5-HT_{1A} receptor subtype has been generated since its discovery in 1981 (Pedigo et al., J. Neurochem. 1981, 36, 220) and subsequent cloning in 1988 (Fargin et al., Nature 1988, 335, 358). In contrast to the SSRIs, a 5-HT_{1A} agonist or partial agonist acts directly on postsynaptic serotonin receptors to increase serotonergic neurotransmission during the latency period for the SSRI effect. Accordingly, the 5-HT_{1A} partial agonists, buspirone and gepirone [Feiger, A., Psychopharmacol. Bull., 1996, 32(4): 659-665; Wilcox, C., Psychopharmacol. Bull., 1996, 32(93): 335-342], and the 5-HT_{1A} agonist, flesinoxan [Grof, P., International Clinical Psychopharmacology, 1993, 8(3): 167-172], have shown efficacy in clinical trials for the treatment of depression. Furthermore, such agents are believed to stimulate the somatodendritic autoreceptors, thus hastening their desensitization and decreasing the SSRI latency period.

Numerous preclinical studies suggest the potential usefulness of 5-HT_{1A} antagonists in the treatment of various diseases and disorders of the central nervous system (CNS), particularly anxiety and depression. Preclinical and clinical data now indicate that compounds that antagonize 5-HT_{1A} receptors may find use in the treatment, prevention and amelioration of central nervous system diseases and disorders, including anxiety, depression, schizophrenia and cognitive deficits resulting from neurodegenerative disorders like Alzheimer's disease; the enhancement of antidepressant activity; the treatment and amelioration of prostate cancer; and the treatment for smoking cessation and nicotine withdrawal [K. Rasmussen and V. P. Rocco, “Recent Progress in Serotonin (5-HT)_{1A} Receptor Modulators,” Annual Reports in Medicinal Chemistry, vol. 30, J. A. Bristol, ed., 1-9 (1995); L. E. Schechter and M. G. Kelly, “An Overview of 5-HT_{1A} Receptor Antagonists: Historical Perspective and Therapeutic Targets,” Current Drugs Serotonin ID Research Alert 1997, 2, 299-309]. A 5HT_{1A} antagonist would limit the negative feedback and should improve the efficacy of the serotonin reuptake mechanism [Perez, V., et al., The Lancet, 349:1594-1597 (1997)]. Accordingly, it would be desirable to develop new compounds that are capable of binding to 5-HT_{1A} receptors and antagonizing their activity.

An agent with a dual mechanism of antidepressant action would be expected to have greater efficacy and thus reduce the number of patients refractory to treatment. Indeed, buspirone augmentation to standard SSRI therapy has been shown to produce marked clinical improvement in patients initially unresponsive to standard antidepressant therapy [Dimitriou, E., J. Clinical Psychopharmacol., 1998, 18(6): 465-469].

There is still an unfilled need for a single agent with a dual mechanism of antidepressant action, i.e., one that not only inhibits or blocks serotonin reuptake (to increase levels of serotonin in the synapse) but also antagonizes the 5-HT_{1A} receptors (to reduce the latency period). The present invention is directed to these, as well as other important ends.

### SUMMARY OF THE INVENTION

In one aspect, the invention provides compounds having the Formula I:

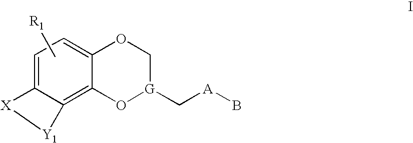

or pharmaceutically-acceptable salt thereof;

wherein:

G is a carbon atom having a hydrogen atom attached thereto, wherein the carbon atom can have either the R or S configuration;
R₁ is hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;

the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, hydroxy, C₁₋₁₂ alkoxy, halo, C₁₋₆ alkyl, amino, or mono- or di-alkylamino in which each alkyl group has 1 to 6 carbon atoms;
R₄ is hydrogen or C₁₋₆ alkyl;
A is -Q₁-Q₂-;
Q₁ is —N(R₈)—,

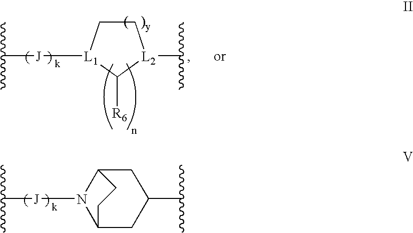

wherein:
J is NH;
k is 0 or 1;
L₁ is N or CH;
L₂ is N or CH;
R₆ is hydrogen or C₁₋₁₂ alkyl;
R₈ is hydrogen, C₁₋₁₂ alkyl C₄₋₂₀ cycloalkylalkyl, or C₃₋₈ cycloalkyl;
n is 1, 2 or 3;
y is 1 or 2, provided that when y is 2, then n is 1;
provided that when k is 1, then L₁ is CH;

Q₂ has the Formula III or IV:

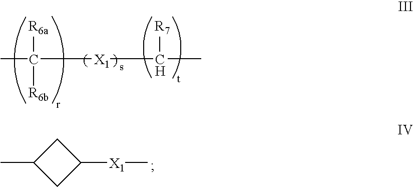

wherein:
each R₆ₐ and each R_{6b} is independently hydrogen or C₁₋₁₂ alkyl;
each R₇ is independently hydrogen or C₁₋₁₂ alkyl;
X₁ is O, NH or —(CH₂)ᵥ— where v is 1, 2, 3 or 4;
r is 0, 1, 2, 3 or 4;
s is 0 or 1;
t is 0, 1, 2 or 3;
provided that when Q₂ has the Formula IV where X₁ is O or (CH₂)ᵥ then Q₁ is N(R₈)—;

B has the Formula:

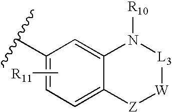

wherein:
R₁₀ is hydrogen, C₁₋₁₂ alkyl or (CH₂)ₓ-cycloalkyl, where x is 4-20;
L₃ is —C(═W′)— or CH₂;
W′ is O, or S ;
W is O, CH₂ or

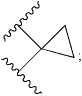

Z is O, CH₂, S, or SO₂;
R₁₁ is hydrogen, C₁₋₁₂ alkyl, cyano or halogen;
provided that Z and W are not both O; and
provided that W is not O when Z is S or SO₂,

In some embodiments, W is CH₂. In some embodiments, W is CH₂ and Z is O. In some embodiments, L₃ is —C(═W′)— wherein W′ is O. In some embodiments, R₁₀ is H or C₁₋₆ alkyl. In some embodiments, R₁₀ is H, CH₃ or C₂H₅. In some embodiments, Z is O. In some embodiments, Z is S. In some embodiments, Z is SO₂. In some embodiments, Z is CH₂. In some embodiments, Z is O; W is CH₂; and L₃ is —C(═W′)— wherein W′ is O.

In some embodiments, Z is O; W is CH₂; L₃ is —C(═W′)— wherein W′ is O; and R₁₀ is hydrogen or C₁₋₆ alkyl. In some of these embodiments, wherein R₁₁ is hydrogen.

In some embodiments, the group X—Y₁ is —N═C(R₂)—C(R₃)═N—.

In some embodiments, the group X—Y₁ is —N═C(R₂)—C(R₄)═CH—.

In some embodiments, the group X—Y₁ is —N═C(R₂)—N═CH—.

In some embodiments, the group X—Y₁ is —N═C(R₂)—O—.

In some embodiments, Z is O; W is CH₂; L₃ is —C(═W′)— wherein W′ is O; and the group X—Y₁ is —N═C(R₂)—C(R₄)═CH—. In some of these embodiments, R₁₀ is hydrogen or C₁₋₆ alkyl; and R₁₁ is hydrogen. In some embodiments, R₂ is methyl, and R₄ is hydrogen. In some embodiments, R₁₀ is hydrogen or C₁₋₆ alkyl; R₁₁ is hydrogen; R₂ is methyl; and R₄ is hydrogen. In some embodiments, Q₁ has the Formula II; and Q₂ has the Formula III. In some embodiments, k is 0; L₁ is N; L₂ is CH—; each R₆ is hydrogen; r and t are each 0; s is 1; and X₁ is O or CH₂. In some embodiments, k is 0; L₁ is N; L₂ is CH; each R₆ is hydrogen; r is 1; t is 0; s is 1; and X₁ is O or CH₂. In some embodiments, k is 0; L₁ is N; L₂ is N; n is 1 or 2; each R₆ is hydrogen; r is 1; s is 0; and t is 0. In some embodiments, k is 0; L₁ is N; L₂ is N; n is 2; r is 1; s is 0; and t is 0. In some embodiments, each of said R_{6b} is hydrogen; one of said R₆ₐ is hydrogen, and the other of said R₆ₐ is C₁₋₆ alkyl. In some embodiments, k is 0; L₁ is N; L₂ is N; n is 1 or 2; each R₆ is hydrogen; and r is 0; s is 0; and t is 0. In some embodiments, k is 1; L₁ is CH; L₂ is N; n is 1; R₆ is hydrogen; r is 1, 2 or 3; each R₆ₐ and each R_{6b} is hydrogen; s is 0; and t is 0. In some embodiments, k is 0; L₁ is N; L₂ is CH; n is 1; R₆ is hydrogen; r is 0; s is 1; X₁ is NH; t is 1, 2 or 3; and each R₇ is hydrogen.

In some embodiments, wherein Q₁ is —N(R₈)—. In some of these embodiments, Q₂ has the Formula IV, wherein X₁ is O or CH₂. In some embodiments, Q₂ has the Formula III, wherein r is 2, 3, or 4; s is 1; X₁ is NH; and t is 1. In some embodiments, Q₂ has the Formula III, wherein r is 3; s is 1; X₁ is O; and t is 0. In some embodiments, Q₂ has the Formula III, wherein r is 2; s is 1; X₁ is O; and t is 0. In some embodiments, Q₂ has the Formula III, wherein r is 3; s is 0; and t is 0. In some embodiments, Q₂ has the Formula III, wherein r is 2, 3 or 4; s is 0; and t is 0. In some embodiments, Q₂ has the Formula III, wherein r is 3 or 4; s is 1; X₁ is O; and t is 0.

In some embodiments, wherein Q₁ has the Formula

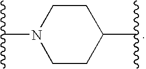

In some embodiments, Q₂ is O, CH₂ or CH₂CH₂. In some embodiments, R₁₀ is hydrogen or C₁₋₆ alkyl. In some embodiments, R₂ is methyl, and R₄ is hydrogen.

In some embodiments, wherein Q₁ has the formula

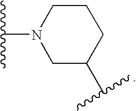

In some embodiments, Q₂ is O, CH₂ or CH₂CH₂. In some embodiments, R₁₀ is hydrogen or C₁₋₆ alkyl. In some embodiments, R₂ is methyl, and R₄ is hydrogen.

In some embodiments, Q₁ has the formula

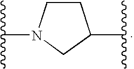

In some embodiments, Q₂ is O, CH₂ or CH₂CH₂. In some embodiments, R₁₀ is hydrogen or C₁₋₆ alkyl. In some embodiments, R₂ is methyl, and R₄ is hydrogen.

In some embodiments, Q₂ has the Formula III, wherein r is 3; s is 1; X₁ is O; and t is 0. In some embodiments, R₁₀ is hydrogen or C₁₋₆ alkyl. The compound of claim 52, wherein R₂ is methyl, and R₄ is hydrogen.

In some embodiments, Q₂ has the Formula III, wherein r is 4; s is 1; X₁ is O; and t is 0. In some embodiments, R₁₀ is hydrogen or C₁₋₆ alkyl. In some embodiments, R₂ is methyl, and R₄ is hydrogen.

In some embodiments, the carbon atom of said G has the S configuration. In some embodiments, the compounds is substantially free of the R enantiomer of said compound.

In some embodiments, compounds of the invention have one of the following Formulas:

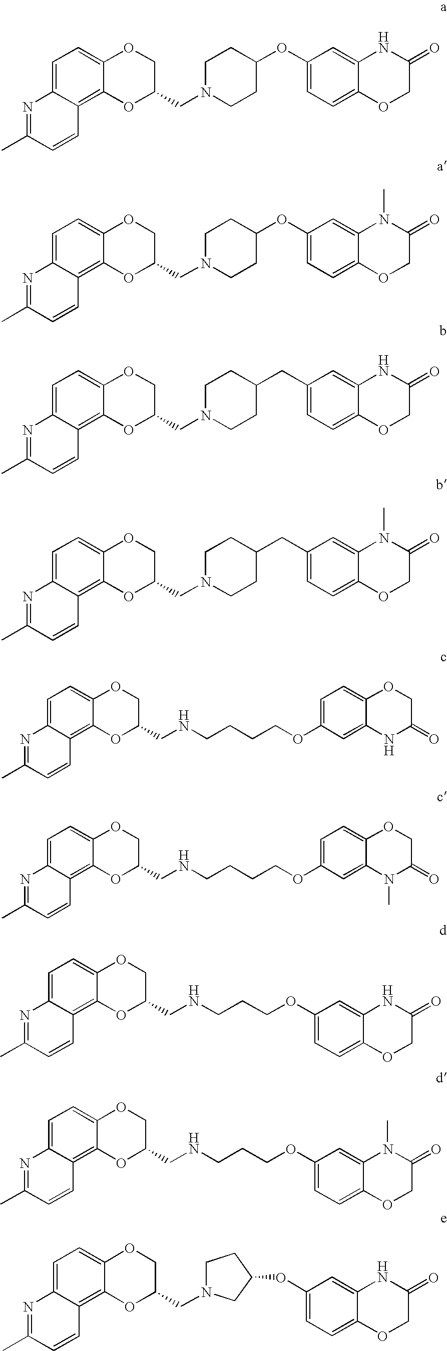

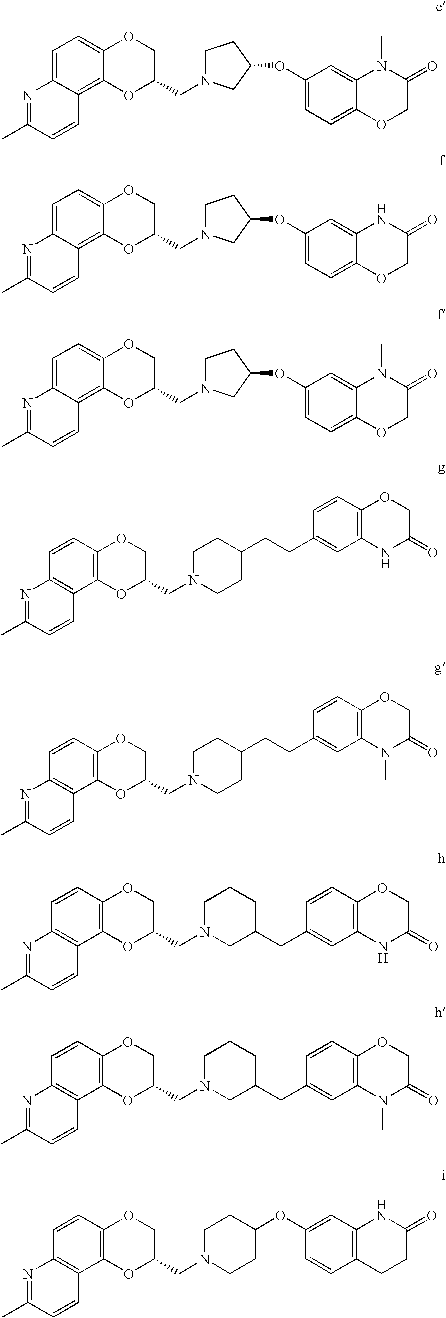

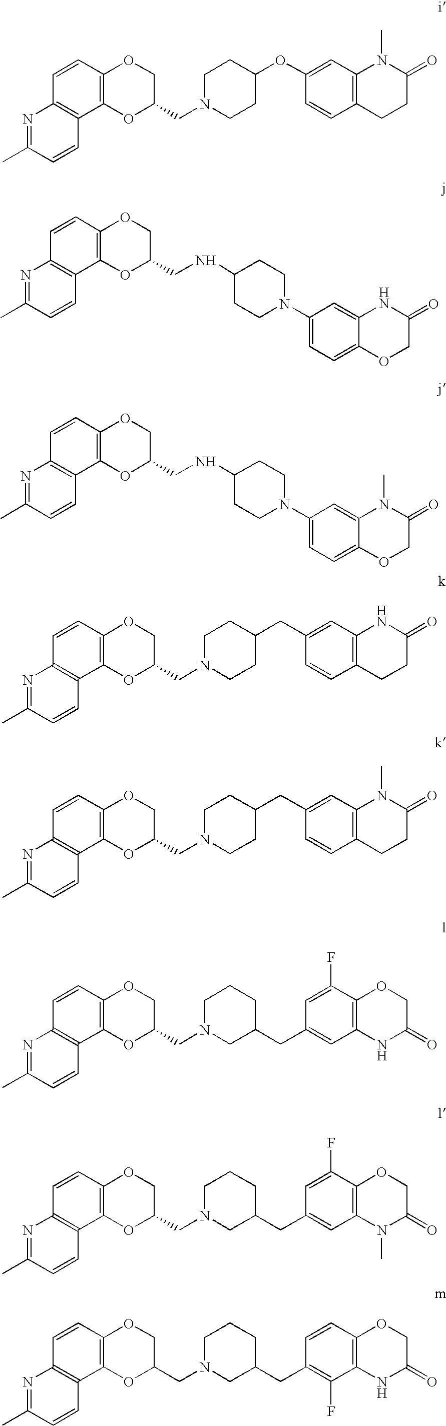

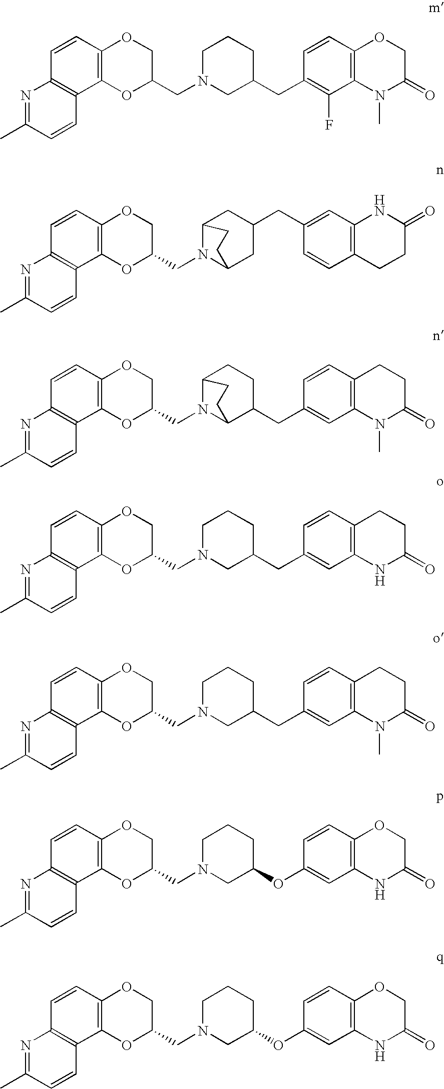

In some embodiments, a method is disclosed for treating a subject suffering from a condition selected from depression, anxiety, panic disorder, post-traumatic stress disorder, premenstrual dysphoric disorder, attention deficit disorder, obsessive compulsive disorder, social anxiety disorder, generalized anxiety disorder, obesity, eating disorders, vasomotor flushing, cocaine and alcohol addiction, and sexual dysfunction, comprising the step of:
administering to said subject suffering from said condition, a therapeutically effective amount of a compound according to claim 1, or a pharmaceutically acceptable salt thereof.

In some embodiments, the condition is depression. In some embodiments, the condition is selected from the group consisting of obsessive-compulsive disorder, panic attacks, generalized anxiety disorder, and social anxiety disorder.

In some embodiments, a pharmaceutical composition, comprises an effective amount of a compound as described herein, or a pharmaceutically acceptable salt thereof; and a pharmaceutically acceptable carrier or excipient.

In some embodiments, a method of making compounds of the invention comprises the step of:
a) reacting a compound of Formula S1:

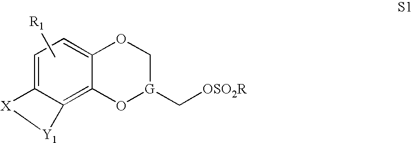

wherein:

G is a carbon atom having a hydrogen atom attached thereto, wherein the carbon atom can have either the R or S configuration;
R is a an appropriately substituted phenyl moiety;
R₁ is hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;
the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, halo, C₁₋₆ alkyl, amino, or mono- or di-alkylamino in which each alkyl group has 1 to 6 carbon atoms;
R₄ is hydrogen or C₁₋₆ alkyl;
   with a compound of Formula S2:

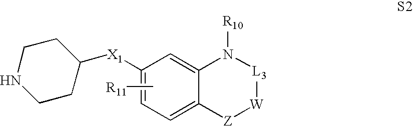

wherein:
R₁₀ is hydrogen, C₁₋₁₂ alkyl or (CH₂)ₓcycloalkyl, where x is 4-20;
L₃ is —C(═W′)— or CH₂;
W′ is O or S;
W is O, CH₂ or

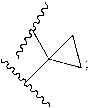

Z is O, CH₂ S, or SO₂;
R₁₁ is hydrogen, C₁₋₁₂ alkyl, cyano or halogen;
provided that Z and W are not both O; and
provided that W is not O when Z is S or SO₂;
   for a time and under conditions effective to form a compound of Formula S3:

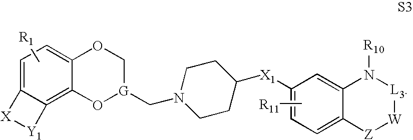

In some embodiments, Z is O, W is CH₂, and L₃ is —C(═W′)— wherein W′ is O. In some embodiments, X—Y₁ is —N═C(R₂)—C(R₄)═CH—. In some embodiments, Z is O, W is CH₂, L₃ is —C(═W′)— wherein W′ is O, and X—Y₁ is —N═C(R₂)—C(R₄)═CH—.

In some embodiments, the compound of Formula S2 is prepared by a method comprising the steps of:

(a1) reacting a compound of Formula S4:

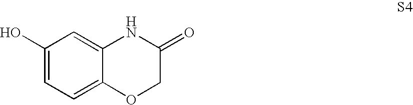

or a salt thereof;
with a compound of Formula S5:

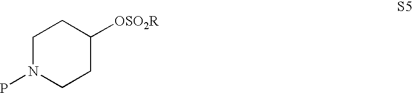

wherein P is an amino protecting group;
for a time and under conditions effective to form a compound of Formula S6:

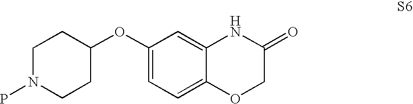

and

(a2) removing the protecting group P to provide the compound of Formula S2.

In some embodiments, the sodium salt of the compound of Formula S4 is reacted with the compound of Formula S5 in an aprotic solvent. In some embodiments, the reaction of the compound of Formula S4 and the compound of Formula S5 are reacted in an aprotic solvent, in the presence of an inorganic base (e.g., cesium carbonate). In some embodiments, the compound of Formula S4 is prepared by reacting a compound of Formula S7

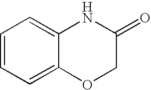

with PIFA in TFA.

In some embodiments, the compound of Formula S5 is prepared by reacting a compound of Formula S8

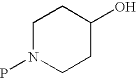

with tosyl chloride or nosyl chloride.

In some embodiments, the compound of Formula S2 is prepared by a method comprising the steps of:

(b2) reacting a compound of Formula S9

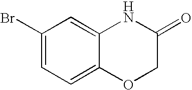

with a compound of Formula S8

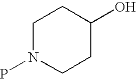

wherein P is an amino protecting group, for a time and under conditions effective to form a compound of Formula S6:

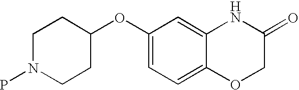

and

(a2) removing the protecting group P to provide the compound of Formula S2.

In some embodiments, the compound of formula S9 is prepared by a method comprising the steps of:

(b1) reacting a compound of Formula S7

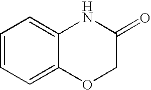

with Br₂ in acetic acid to form a compound of formula S9.

In some embodiments, the compound of formula S9 is prepared by a method comprising the steps of alkylating 4-bromo-2-nitrophenol with methyl bromoacetate; to produce an intermediate ether, and reducing and cyclizing said intermediate ether to the desired compound of formula S9.

In some embodiments, the compound of Formula S2 is prepared by a method comprising the steps of:

(c1) reacting a compound of Formula S4

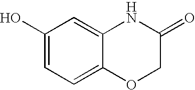

with a compound of Formula S8

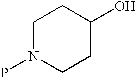

for a time and under conditions effective to form the compound of Formula S6

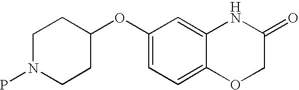

and

(c2) removing the protecting group P to provide the compound of Formula S2.

In some embodiments, the reacting of the compound of Formula S4 and S8 is performed under Mitsunobu conditions.

In some embodiments, reacting of the compound of Formula S4 and S8 is performed under Williamson conditions.

In some embodiments, a method of making compounds of the invention comprises the step of:

a) reacting a compound of Formula S10:

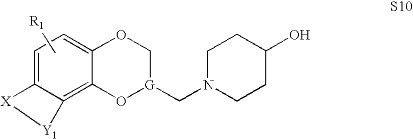

wherein:

G is a carbon atom having a hydrogen atom attached thereto, wherein the carbon atom can have either the R or S configuration;
R₁ is hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;
the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, halo, C₁₋₆ alkyl, amino, or mono- or di-alkylamino in which each alkyl group has 1 to 6 carbon atoms;
R₄ is hydrogen or C₁₋₆ alkyl;
   with a compound of Formula S11:

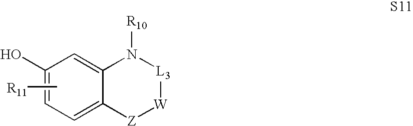

wherein:
R₁₀ is hydrogen, C₁₋₁₂ alkyl or C₄₋₂₀ alkylcycloalkyl;
L₃ is —C(═W′)— or CH₂;
W′ is O or S;
W is O, CH₂ or

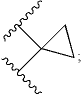

Z is O, CH₂ S or SO₂;
R₁₁ is hydrogen, C₁₋₁₂ alkyl, cyano or halogen;
provided that Z and W are not both O; and
provided that W is not O when Z is S or SO₂;
   for a time and under conditions effective to form a compound of Formula S12:

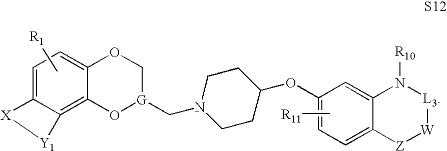

In some embodiments, Z is O, W is CH₂, and L₃ is —C(═W′)— wherein W′ is O. In some embodiments, X—Y₁ is —N═C(R₂)—C(R₄)═CH—. In some embodiments, Z is O, W is CH₂, L₃ is —C(═W′)— wherein W′ is O, and X—Y₁ is —N═C(R₂)—C(R₄)═CH—.

In some embodiments, the compound of Formula S10 is prepared by a method comprising the step of:

(d1) reacting a compound of Formula S1:

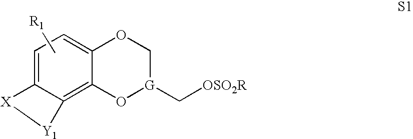

wherein R is a an appropriately substituted phenyl moiety;

with 4-hydroxypiperidine, for a time and under conditions effective to form the compound of Formula S10.

In some embodiments, a method of preparing a compound of formula I

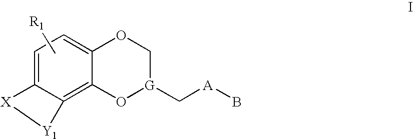

or pharmaceutically-acceptable salt thereof;

wherein:

G is a carbon atom having a hydrogen atom attached thereto, wherein the carbon atom can have either the R or S configuration;
R₁ is hydrogen, hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;

the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, hydroxy, C₁₋₁₂ alkoxy, halo, C₁₋₆ alkyl, amino, or mono- or di-alkylamino in which each alkyl group has 1 to 6 carbon atoms;
R₄ is hydrogen or C₁₋₆ alkyl;
A is -Q₁-Q₂-;

Q₁ is a group of Formula II:

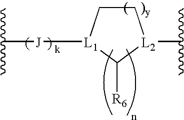

wherein:
J is NH;
k is 1;
; L₁ is CH;
L₂ is N;
R₆ is hydrogen;
R₈ is hydrogen, C₁₋₁₂ alkyl C₄₋₂₀ alkylcycloalkyl, or C₃₋₈ cycloalkyl;
n is 2;
y is 1;

Q₂ has the Formula III

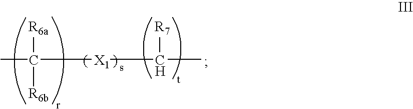

wherein:
each R₆ₐ and each R_{6b} is independently hydrogen or C₁₋₁₂ alkyl;
each R₇ is independently hydrogen or C₁₋₁₂ alkyl;
X₁ is O, NH or —(CH₂)ᵥ— where v is 1, 2, 3 or 4;
r is 0;
s is 0;
t is 0;
provided that when when Q₂ has the Formula IV where X₁ is O or (CH₂)ᵥ then Q₁ is N(R₈);

B has the Formula:

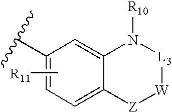

wherein:
R₁₀ is hydrogen, C₁₋₁₂ alkyl or C₄₋₂₀ alkylcycloalkyl, (CH₂)ₓ-cycloalkyl;
L₃ is —C(═W′)— or CH₂;
W′ is O or S;
W is O, CH₂ or

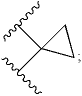

Z is O, CH₂ S or SO₂;
R₁₁ is hydrogen, C₁₋₁₂ alkyl, cyano or halogen;
provided that Z and W are not both O; and
provided that W is not O when Z is S or SO₂;
   comprising the steps of:

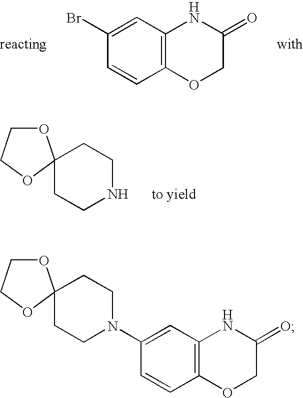

treating the resultant product with acid to yield

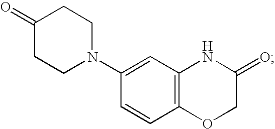

performing reductive amination with a compound of formula

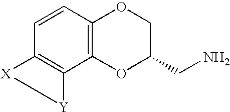

to produce a compound of formula 1.

In some such embodiments, —X—Y— is —N═C(R₂)—C(R₄)═CH—. In some embodiments, —X—Y— is —N═C(R₂)—C(R₄)═CH— and R₂ is methyl.

### DESCRIPTION OF THE INVENTION

Some embodiments of the invention provide novel compounds of Formula I:

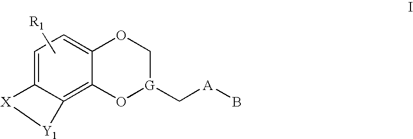

or pharmaceutically-acceptable salts thereof;

wherein:

G is a carbon atom having a hydrogen atom attached thereto, wherein the carbon atom can have either the R or S configuration;
R₁ is hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;
the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, hydroxy, C₁₋₁₂ alkoxy, halo, C₁₋₆ alkyl, amino, or mono- or di-alkylamino in which each alkyl group has 1 to 6 carbon atoms;
R₄ is hydrogen or C₁₋₆ alkyl;
A is -Q₁-Q₂-;
Q₁ is —N(R₈)—,

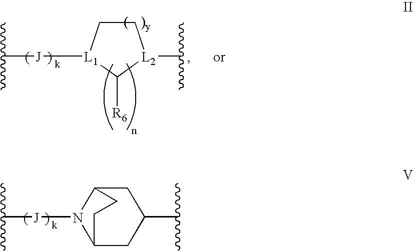

wherein:
J is NH;
k is 0 or 1;
L₁ is N or CH;
L₂ is N or CH;
R₆ is hydrogen or C₁₋₁₂ alkyl;
R₈ is hydrogen, C₁₋₁₂ alkyl C₄₋₂₀ cycloalkylalkly, or C₃₋₈ cycloalkyl;
n is 1, 2 or 3;
y is 1 or 2; provided that when y is 2, n is 1;
provided that when k is 1, then L₁ is CH;

Q₂ has the Formula III or IV:

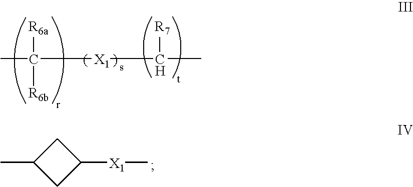

wherein:
each R₆ₐ and each R_{6b} is independently hydrogen or C₁₋₁₂ alkyl;
each R₇ is independently hydrogen or C₁₋₁₂ alkyl;
X₁ is O, NH or —(CH₂)ᵥ— where v is 1, 2, 3 or 4;
r is 0, 1, 2, 3 or 4;
s is 0 or 1;
t is 0, 1, 2 or 3;
provided that when Q₂ has the Formula IV where X₁ is O or (CH₂)ᵥ then Q1 is N(R₈);

B has the Formula:

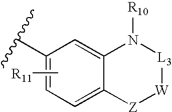

wherein:
R₁₀ is hydrogen, C₁₋₁₂ alkyl or (CH₂)ₓ-cycloalkyl, where x is 4-20;
L₃ is —C(═W′)— or CH₂;
W′ is O or S;
W is O, CH₂ or

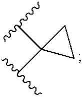

Z is O, CH₂, S or SO₂;
R₁₁ is hydrogen, C₁₋₁₂ alkyl, cyano or halogen;
provided that Z and W are not both O; and
provided that W is not O, when Z is S or SO₂.

In some embodiments, W is CH₂. In some embodiments, W is CH₂ and Z is O. In some embodiments, L₃ is —C(═W′)— wherein W′ is O. In some further embodiments, R₁₀ is H or C₁₋₆ alkyl, for example CH₃ or C₂H₅. In some embodiments, Z is O. In some embodiments, Z is S. In some embodiments, Z is CH₂. In some further embodiments, Z is O, W is CH₂ and L₃ is —C(═W′)— wherein W′ is O. In some further embodiments, Z is O, W is CH₂, L₃ is —C(═W′)— wherein W′ is O, and R₁₀ is hydrogen or C₁₋₆ alkyl. In some of the foregoing embodiments, R₁₁ is hydrogen.

In some embodiments, Z is O, W is CH₂, L₃ is —C(═W′)— wherein W′ is O and the group X—Y₁ is —N═C(R₂)—C(R₄)═CH—. In some such embodiments, R₁₀ is hydrogen or C₁₋₆ alkyl, and R₁₁ is hydrogen. In some such embodiments, R₂ is methyl and R₄ is hydrogen.

In some embodiments, Q₁ has the Formula II, and Q₂ has the Formula III. In some such embodiments, k is 0, L₁ is N, L₂ is CH, each R₆ is hydrogen, r and t are each 0, s is 1 and X₁ is O or CH₂. In some further embodiments, k is 0, L₁ is N, L₂ is CH, each R₆ is hydrogen, r is 1, t is 0, s is 1 and X₁ is O or CH₂. In some further embodiments, k is 0, L₁ is N, L₂ is N, n is 1 or 2, each R₆ is hydrogen, r is 1, s is 0 and t is 0.

In some further embodiments wherein Q₁ has the Formula II and Q₂ has the Formula III, k is 0, L₁ is N, L₂ is N, n is 2, r is 1, s is 0 and t is 0. In some such embodiments, each R_{6b} is hydrogen, and one R₆ₐ is hydrogen and the other R₆ₐ is C₁₋₆ alkyl.

In some further embodiments wherein Q₁ has the Formula II and Q₂ has the Formula III, k is 0, L₁ is N, L₂ is N, n is 1 or 2, each R₆ is hydrogen, and r, s and t are each 0 (that is, Q₂ is a bond). In still further such embodiments, k is 0, L₁ is N, L₂ is CH, n is 1, R₆ is hydrogen, r is 0, s is 1, X₁ is NH, t is 1, 2 or 3, and each R₇ is hydrogen.

In some embodiments, Q₁ is —N(R₈)—, and Q₂ has the Formula IV, wherein X₁ is O or (CH₂)ᵥ.

In some embodiments, Q₁ is —N(R₈)—, and Q₂ has the Formula III. In some such embodiments: r is 2, 3, or 4, s is 1, X₁ is NH, and t is 1; or r is 3, s is 1, X₁ is O and t is 0; or r is 2, s is 1, X₁ is O and t is 0; or r is 3, s is 0 and t is 0; or r is 2, 3 or 4, s is 0,and t is 0; or r is 3 or 4, s is 1, X₁ is O and t is 0.

In some embodiments, Q₁ has one of the Formulas:

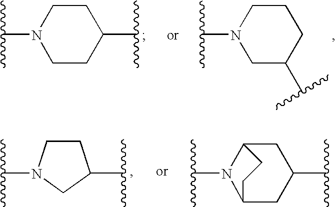

In some such embodiments, Q₂ is O, CH₂ or CH₂CH₂.

In some embodiments, Q₁ is —N(R₈)—, and Q₂ has the Formula III, wherein: r is 3, s is 1, X₁ is O and t is 0; or r is 4, s is 1, X₁ is O and t is 0. In some such embodiments, R₁₀ is hydrogen or C₁₋₆ alkyl. In some further such embodiments, R₂ is methyl, and R₄ is hydrogen.

In some of each of the foregoing embodiments, the carbon atom of G has the S configuration. In some further embodiments, the compound is substantially free of the R enantiomer of the compound.

The compounds of the invention are useful for the treatment of a variety of conditions including depression, anxiety, panic disorder, post-traumatic stress disorder, premenstrual dysphoric disorder, attention deficit disorder, obsessive compulsive disorder, social anxiety disorder, generalized anxiety disorder, obesity, eating disorders, vasomotor flushing, cocaine and alcohol addiction, and sexual dysfunction. Thus, the invention provides methods for the treatment of such conditions, that include the administration of a compound of the invention, or a pharmaceutical composition containing a compound of the invention, to an individual suffering from such condition. In some preferred embodiments, the condition is depression. In other preferred embodiments, the condition is obsessive-compulsive disorder, panic attacks, generalized anxiety disorder or social anxiety disorder.

Some embodiments of the invention further provides pharmaceutical compositions that include an effective amount of a compound of the invention, or a pharmaceutically acceptable salt thereof; and one or more pharmaceutically acceptable carriers or excipients.

Various embodiments of the invention relate to both the individual R and S stereoisomers of compounds of Formula I, as well as to mixtures of the R and S stereoisomers. Throughout this application, unless the absolute configuration of a compound of the invention is specifically indicated, names and structural formulas of compounds of the invention are intended to include both R and S enantiomers, as well as mixtures of the two. In some embodiments of the present invention, compounds wherein the carbon atom designated “G” in Formula I have the S configuration are preferred. Certain of the compounds of this invention can contain two stereogenic centers and thus may exist as diastereomers. This invention relates to both diastereomers, as well as to mixtures of diastereomers.

In some embodiments, the compounds of the invention exist or are provided substantially free of one enantiomer. As used herein, “substantially free” of a given enantiomer means that the desired enantiomer is present in an amount that is at least about 90% by weight of the compound. In some embodiments, the preferred enantiomer is present in an amount that is at least about 95% by weight of the compound; or at least about 98% by weight of the copmpound, or at least about 99% by weight of the compound. Preferred stereoisomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

In some embodiments, the compounds and pharmaceutically acceptable salts of compounds of the present invention exist as tautomers. Such tautomers can be transient or isolable as a stable product. These tautomers are within the scope of the compounds described herein. For example, when W′ is O and R₁₀ is hydrogen, two tautomeric forms of the compounds of Formula I; both are within the scope of the invention, as well as mixtures of the two.

“Alkyl,” as used herein, refers to an aliphatic hydrocarbon chain and includes straight and branched chains such as methyl, ethyl, n-propyl, isopropyl, n-butyl, isobutyl, sec-butyl, t-butyl, n-pentyl, isopentyl, neo-pentyl, n-hexyl, and isohexyl. In some embodiments, alkyl groups have from 1 to 20, or from 1 to 12, or from 1 to 6 carbon atoms. The term “lower alkyl” refers to alkyl groups having 1 to 3 carbon atoms.

“Alkanamido,” as used herein, refers to the group R—C(═O)—NH— where R is an alkyl group of 1 to 5 carbon atoms.

“Alkanoyl,” as used herein, refers to the group R—C(═O)— where R is an alkyl group of 1 to 5 carbon atoms.

—Alkanoyloxy,— as used herein, refers to the group R—C(═O)—O— where R is an alkyl group of 1 to 5 carbon atoms.

“Alkanesulfonamido,” as used herein, refers to the group R—S(O)₂—NH— where R is an alkyl group of 1 to 6 carbon atoms.

“Alkanesulfonyl,” as used herein, refers to the group R—S(O)₂— where R is an alkyl group of 1 to 6 carbon atoms.

“Alkoxy,” as used herein, refers to the group R—O— where R is an alkyl group of 1 to 20, or from 1 to 12, or from 1 to 6 carbon atoms.

“Alkylcycloalkyl” as used herein, refers to a cycloalkyl group that has appended thereto one or more alkyl groups. In some embodiments, alkylcycloalkyl groups have from 4 to 20 carbon atoms, or from 4 to 12 carbon atoms.

“Cycloalkylalkyl” as used herein, refers to an alkyl group that has a cycloalkyl group appended thereto. In some embodiments, cycloalkylalkyl groups have from 4 to 20 carbon atoms, or from 4 to 12 carbon atoms.

“Carboxamido,” as used herein, refers to the group NH₂—C(═O)—.

“Carboalkoxy,” as used herein, refers to the group R—O—C(═O)— where R is an alkyl group of 1 to 5 carbon atoms.

“Cycloalkyl, as used herein, refers to a cyclic alkyl group having from 3 to 8 ring carbon atoms, i.e., cyclopropyl, cyclobutyl, cyclopentyl, cyclohexyl, cycloheptyl and cyclooctyl groups.

“Halogen” (or “halo”), as used herein, refers to chlorine, bromine, fluorine and iodine.

The term “pharmaceutically acceptable salt”, as used herein, refers to salts derived from organic and inorganic acids of a compound described herein. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, cinnamate, glycolate, pyruvate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate. The term “pharmaceutically acceptable salt” also includes hydrates of a compound described herein. In some embodiments, fumarate salts are employed.

The term “Williamson conditions,” as used herein, refers to the conditions set forth in H. C. Aspinall, et al., “An improved Williamson etherification of hindered alcohols promoted by 15-crown-5 and sodium hydroxide,” Tetrahedron Letters, Vol. 38, No. 26, pp. 4679-4682, 1997, which is hereby incorporated by reference in its entirety.

Specific examples of compounds of Formula I include compounds a-q:

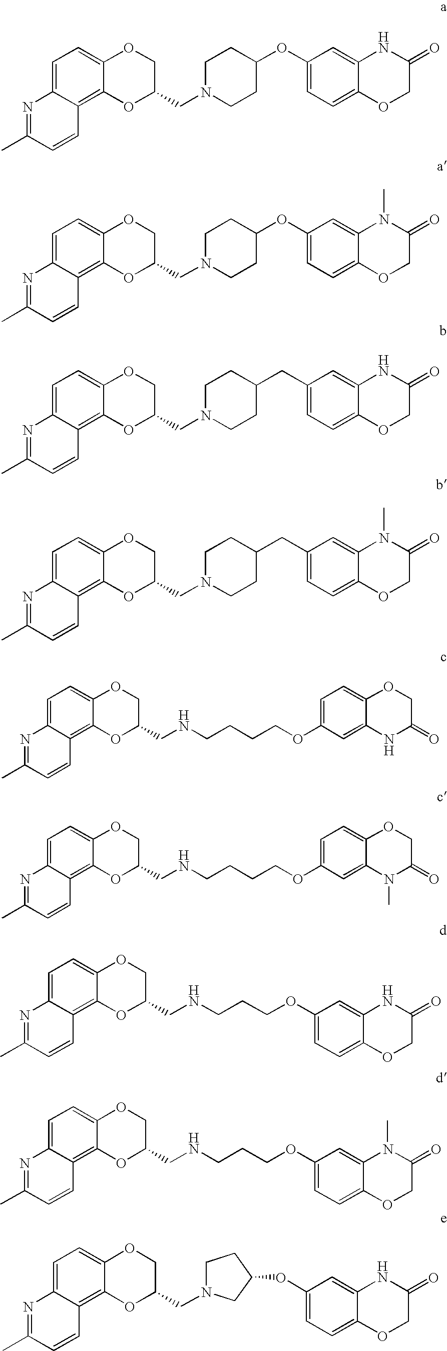

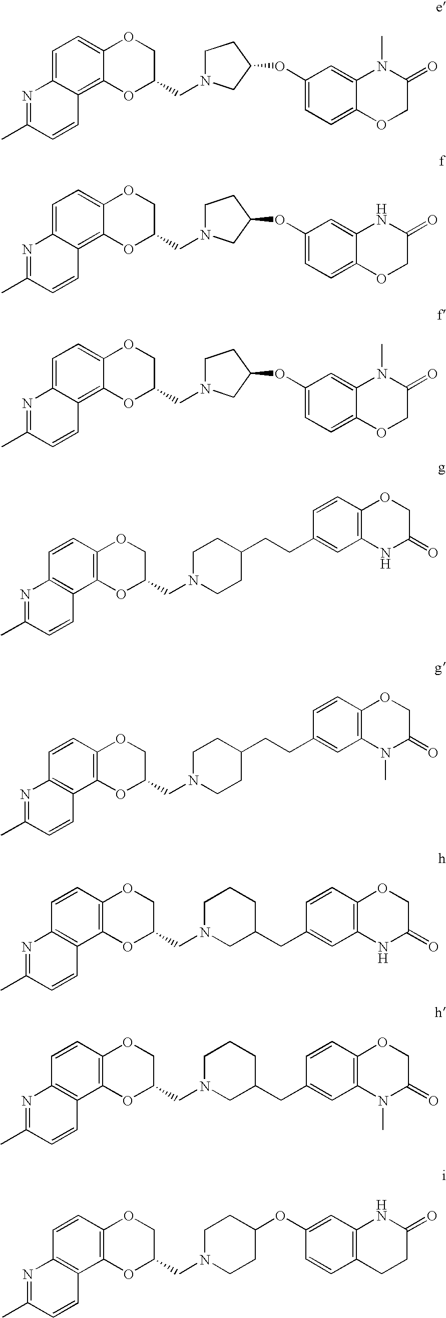

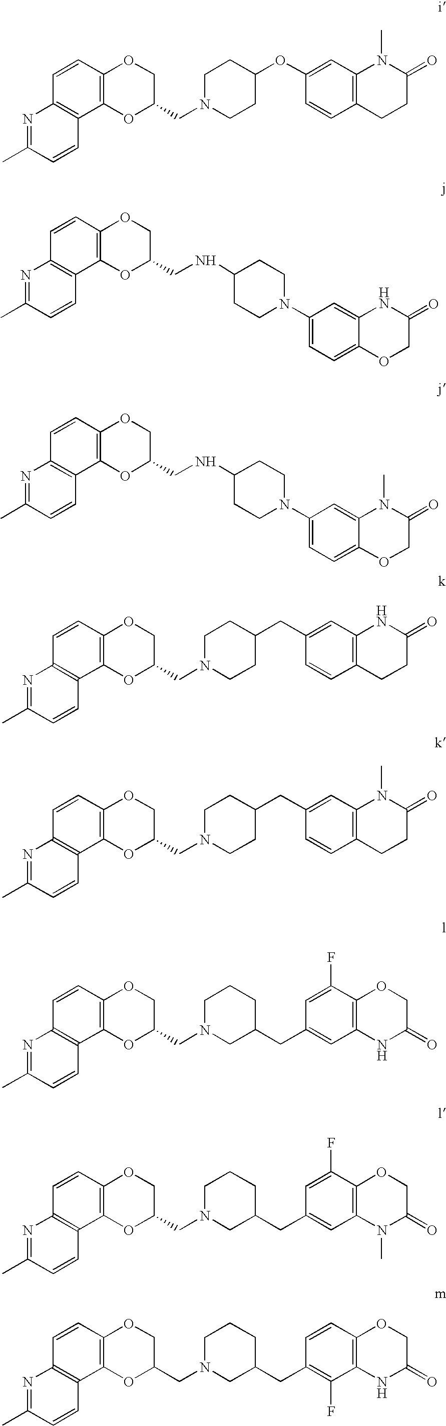

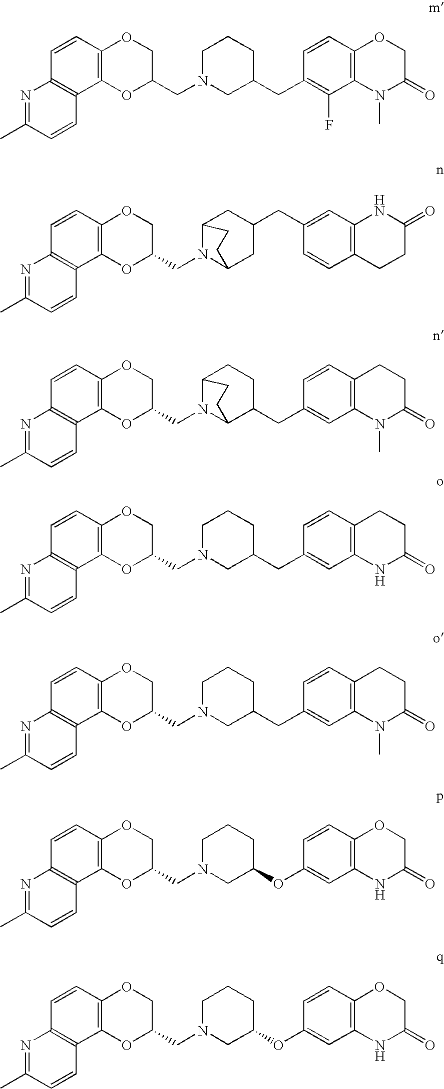

and pharmaceutically acceptable salts thereof.

Like the antidepressants fluoxetine, paroxetine and sertraline, the compounds of this invention have the ability to potently block the reuptake of the brain neurotransmitter serotonin. Some embodiments of the invention are thus useful for the treatment of diseases commonly treated by the administration of serotonin selective reuptake inhibitor (SSRI) antidepressants, such as depression (including but not limited to major depressive disorder, childhood depression and dysthymia), anxiety, panic disorder, post-traumatic stress disorder, premenstrual dysphoric disorder (also known as pre-menstrual syndrome), attention deficit disorder (with and without hyperactivity), obsessive compulsive disorders (including but not limited to trichotillomania), obsessive compulsive spectrum disorders (including but not limited to autism), social anxiety disorder, generalized anxiety disorder, obesity, eating disorders such as anorexia nervosa, bulimia nervosa, vasomotor flushing, cocaine and alcohol addiction, sexual dysfunction (including but not limited to premature ejaculation), incontinence (including, but not limited to fecal incontinence, urge incontinence, overflow incontinence, passive incontinence, reflex incontinence, stress urinary incontinence urinary exertional incontinence and urinary incontinence), and pain (including, but not limited to migraine, chronic back pain, phantom limb pain, neuropathic pain such as diabetic neuropathy, and post herpetic neuropathy) and related illnesses. Moreover, the compounds of this invention have potent affinity for and antagonist activity at brain 5HT_{1A} serotonin receptors. Recent clinical trials employing drug mixtures (e.g., fluoxetine and pindolol) have demonstrated a more rapid onset of antidepressant efficacy for a treatment combining SSRI activity and 5HT_{1A} antagonism (Blier and Bergeron, J. Clin. Psychopharmacol., 1995, 15 (3): 217-222; F. Artigas et. al., Trends Neurosci. 1996, 19 (9); 378-383; M. B. Tome et. al., J. Affec. Disord., 1997, 44(2-3): 101-109). The compounds of the invention are thus exceedingly interesting and useful for treating depressive illnesses.

Thus, some embodiments of the present invention provides methods of treating, preventing, inhibiting or alleviating each of the maladies listed above in a mammal, preferably in a human, the methods comprising providing a pharmaceutically effective amount of a compound of this invention to the mammal in need thereof.

Also encompassed by some embodiments of the present invention are pharmaceutical compositions for treating or controlling disease states or conditions of the central nervous system comprising at least one compound of Formula I, mixtures thereof, and or pharmaceutical salts thereof, and a pharmaceutically acceptable carrier therefore. Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

In some embodiments, the compounds or pharmaceutically acceptable salts of the compounds described herein are a component of a composition that comprises one or more pharmaceutically acceptable vehicles, carriers, excipients, or diluents. Such pharmaceutical compositions can be prepared using a method comprising admixing the compound or pharmaceutically acceptable salt of the compound and a physiologically acceptable carrier, excipient, or diluent. Admixing can be accomplished using methods well known for admixing a compound or a pharmaceutically acceptable salt of a compound and a physiologically acceptable carrier, excipient, or diluent. Examples of such carriers, excipients, and diluents are well known to those skilled in the art and are prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers, excipients, and diluents are those that are compatible with the other ingredients in the formulation and biologically acceptable.

Exemplary physiologically acceptable excipients include, without limitation, liquids such as water and oils, including those of petroleum, animal, vegetable, or synthetic origin, such as peanut oil, soybean oil, mineral oil, sesame oil and the like. Other nonlimiting examples of physiologically acceptable excipients are saline, gum acacia, gelatin, starch paste, talc, keratin, colloidal silica, urea and the like. In addition, auxiliary, stabilizing, thickening, lubricating, and coloring agents can optionally be used. In one embodiment the physiologically acceptable excipients are sterile when administered to an animal. The physiologically acceptable excipient should be stable under the conditions of manufacture and storage and should be preserved against the contaminating action of microorganisms. Water is a particularly useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include, without limitation, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, also optionally contain minor amounts of wetting or emulsifying agents, or pH buffering agents. Other examples of suitable physiologically acceptable excipients are described in Remington's Pharmaceutical Sciences 1447-1676 (Alfonso R. Gennaro, ed., 19th ed. 1995).

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule.

The compounds or pharmaceutically acceptable salts of the compounds described herein may be administered orally or parenterally, neat or in combination with conventional pharmaceutical carriers as described above. The compounds or pharmaceutically acceptable salts of the compounds described herein can also be administered by any convenient route, for example, orally, by infusion or bolus injection, by absorption through epithelial or mucocutaneous linings (e.g., oral, rectal, vaginal, and intestinal mucosa, etc.) and can be administered together with another therapeutic agent. Other methods of administration include, but are not limited to, intradermal, intramuscular, intraperitoneal, intravenous, subcutaneous, intranasal, epidural, sublingual, intracerebral, intravaginal, transdermal, rectal, by inhalation, or topical, particularly to the ears, nose, eyes, or skin. Administration can be systemic or local. Various known delivery systems, including encapsulation in liposomes, microparticles, microcapsules, and capsules, can be used. For example, in one embodiment, the compounds or pharmaceutically acceptable salts of the compounds described herein can be delivered in a vesicle, in particular a liposome (see Langer, Science 249:1527-1533 (1990) and Treat et al., Liposomes in the Therapy of Infectious Disease and Cancer 317-327 and 353-365 (1989)). In some instances, administration will result of release of the compound or a pharmaceutically acceptable salt of the compound into the bloodstream. The mode of administration is left to the discretion of the practitioner.

In one embodiment, the compound or pharmaceutically acceptable salt of the compound is administered orally.

In another embodiment, the compound or pharmaceutically acceptable salt of the compound is administered intravenously.

In another embodiment, it may be desirable to administer the compounds or pharmaceutically acceptable salts of the compounds described herein locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the compounds or pharmaceutically acceptable salts of the compounds described herein into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

In one embodiment, the compound or a pharmaceutically acceptable salt of the compound is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, elixirs, oral liquids, suspensions or solutions, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Oral formulations may utilize standard delay or time release formulations to alter the absorption of the compound or pharmaceutically acceptable salt of the compound. The oral formulation may also consist of administering the compound or pharmaceutically acceptable salt of the compound in water or fruit juice, containing appropriate solubilizers or emulisifiers as needed.

Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. In powders, the carrier is a finely divided solid, which is an admixture with the finely divided compound or pharmaceutically acceptable salt of the compound. In tablets, in some embodiments, the compound or pharmaceutically acceptable salt of the compound is mixed with a carrier having the necessary compression properties in suitable proportions and compacted in the shape and size desired. In some embodiments, the powders and tablets contain up to 99% of the compound or pharmaceutically acceptable salt of the compound.

Capsules may contain mixtures of the compounds or pharmaceutically acceptable salts of the compounds with inert fillers and/or diluents such as pharmaceutically acceptable starches (e.g. corn, potato or tapioca starch), sugars, artificial sweetening agents, powdered celluloses, (such as crystalline and microcrystalline celluloses), flours, gelatins, gums, and other pharmaceutically acceptable fillers and/or diluents known to those of skill in the art.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins). In some embodiments, the surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

The compound or a pharmaceutically acceptable salt of the compound can be administered by controlled-release or sustained-release means or by delivery devices that are known to those of ordinary skill in the art (see, e.g., Goodson, in Medical Applications of Controlled Release, vol. 2, pp. 115-138 (1984)). Such dosage forms can be used to provide controlled- or sustained-release of one or more compounds or pharmaceutically acceptable salts of the compounds using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the compounds or pharmaceutically acceptable salts of the compounds described herein. Thus, in some embodiments, the compounds or pharmaceutically acceptable salts of the compounds are provided as single unit dosage forms suitable for oral administration such as, but not limited to, tablets, capsules, gelcaps, and caplets that are adapted for controlled- or sustained-release. Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et. al., J. Neurosurg. 71:105 (1989)).

Similarly, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

Liquid carriers may be used in preparing solutions, suspensions, emulsions, syrups and elixirs. The compound or pharmaceutically acceptable salt of the compound described herein can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g., cellulose derivatives, such as sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Pulmonary administration can also be employed, e.g., by use of an inhaler or nebulizer, and formulation with an aerosolizing agent, or via perfusion in a fluorocarbon or synthetic pulmonary surfactant. In some embodiments, the compounds or pharmaceutically acceptable salts of the compounds are administered directly to the airways in the form of an aerosol. For administration by intranasal or intrabronchial inhalation, the compounds or pharmaceutically acceptable salts of the compounds may be formulated into an aqueous or partially aqueous solution

The compounds or pharmaceutically acceptable salt of the compounds described herein may be administered parenterally or intraperitoneally. Solutions or suspensions of these compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In some embodiments, the form is sterile and is fluid to the extent that easy syringability exists. Such dosage forms are generally stable under the conditions of manufacture and storage and are preserved against the contaminating action of microorganisms such as bacteria and fungi. In some embodiments, the carrier is a solvent or dispersion medium containing, for example, water, ethanol, polyol (e.g., glycerol, propylene glycol and liquid polyethylene glycol), suitable mixtures thereof, and vegetable oils.

In another embodiment, the compound or a pharmaceutically acceptable salt of the compound can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of the compound or pharmaceutically acceptable salt of the compound. Where the compound or a pharmaceutically acceptable salt of the compound is to be administered by infusion, it can be dispensed, for example, with an infusion bottle containing sterile pharmaceutical grade water or saline. Where the compound or a pharmaceutically acceptable salt of the compound is administered by injection, an ampule of sterile water for injection or saline can be provided so that the ingredients can be mixed prior to administration.

The compounds or pharmaceutically acceptable salts of the compounds described herein can be administered transdermally through the use of a transdermal patch. Transdermal administrations include administrations across the surface of the body and the inner linings of bodily passages including epithelial and mucosal tissues. Such administrations may be carried out using the compounds described herein, or pharmaceutically acceptable salts thereof, in lotions, creams, foams, patches, suspensions, solutions, and suppositories (rectal and vaginal).

Transdermal administration may be accomplished through the use of a transdermal patch containing the compound or pharmaceutically acceptable salt of the compound and a carrier that is inert to the compound, is non-toxic to the skin, and allows delivery of the compound for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the compound or pharmaceutically acceptable salt of the compound may also be suitable. A variety of occlusive devices may be used to release the compound or pharmaceutically acceptable salt of the compound into the blood stream, such as a semi-permeable membrane covering a reservoir containing the compound or pharmaceutically acceptable salt of the compound with or without a carrier, or a matrix containing the compound or pharmaceutically acceptable salt of the compound. Other occlusive devices are known in the literature.

The compounds or pharmaceutically acceptable salts of the compounds described herein may be administered rectally or vaginally in the form of a conventional suppository. Suppository formulations may be made from traditional binders and excipients, including cocoa butter and triglycerides, with or without the addition of waxes to alter the suppository's melting point, and glycerin. Water soluble suppository bases, such as polyethylene glycols of various molecular weights, may also be used.

It is understood that the dosage, regimen and mode of administration of these compounds will vary according to the malady and the individual being treated and will be subject to the judgment of the medical practitioner involved. In some embodiments, administration of one or more of the compounds or pharmaceutically acceptable salts of the compounds described herein begins at a low dose and is increased until the desired effects are achieved.

When administered for the treatment or inhibition of a particular disease state or disorder, it is understood that the effective dosage may vary depending upon the particular compound or pharmaceutically acceptable salt of the compound utilized, the mode of administration, the condition, and severity thereof, of the condition being treated, as well as the various physical factors related to the individual being treated. In therapeutic application, the compounds and pharmaceutically acceptable salts of the compounds described herein are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient.

In one embodiment a controlled- or sustained-release composition comprises a minimal amount of the compound or a pharmaceutically acceptable salt of the compound to treat or prevent a central nervous system disorder in a minimal amount of time. Advantages of controlled- or sustained-release compositions include extended activity of the drug, reduced dosage frequency, and increased compliance by the animal being treated. In addition, controlled or sustained release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of the compound or a pharmaceutically acceptable salt of the compound that promptly produces the desired therapeutic or prophylactic effect, and gradually and continually release other amounts of the compound or a pharmaceutically acceptable salt of the compound to maintain this level of therapeutic or prophylactic effect over an extended period of time. To maintain a constant level of the compound or a pharmaceutically acceptable salt of the compound in the body, the compound or a pharmaceutically acceptable salt of the compound can be released from the dosage form at a rate that will replace the amount of the compound or a pharmaceutically acceptable salt of the compound being metabolized and excreted from the body. Controlled or sustained release of the compound or pharmaceutically acceptable salt of the compound can be stimulated by various conditions, including but not limited to, changes in pH, changes in temperature, concentration or availability of enzymes, concentration or availability of water, or other physiological conditions or compounds.

In certain embodiments, the present invention is directed to prodrugs of the compounds or pharmaceutically acceptable salts of compounds described herein. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

The amount of the compound or a pharmaceutically acceptable salt of the compound delivered is an amount that is effective for treating or preventing a central nervous system disorder. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound or a pharmaceutically acceptable salt of the compound is administered, the effective dosage amounts correspond to the total amount administered.

The amount of the compound or a pharmaceutically acceptable salt of the compound that is effective for treating or preventing a central nervous system disorder will typically range from about 0.001 mg/kg to about 600 mg/kg of body weight per day, in one embodiment, from about 1 mg/kg to about 600 mg/kg body weight per day, in one embodiment, from about 1 mg/kg to about 250 mg/kg body weight per day, in another embodiment, from about 10 mg/kg to about 400 mg/kg body weight per day, in another embodiment, from about 10 mg/kg to about 200 mg/kg of body weight per day, in another embodiment, from about 10 mg/kg to about 100 mg/kg of body weight per day, in one embodiment, from about 10 mg/kg to about 25 mg/kg body weight per day, in another embodiment, from about 1 mg/kg to about 10 mg/kg body weight per day, in another embodiment, from about 0.001 mg/kg to about 100 mg/kg of body weight per day, in another embodiment, from about 0.001 mg/kg to about 10 mg/kg of body weight per day, and in another embodiment, from about 0.001 mg/kg to about 1 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the compound or pharmaceutically acceptable salt of the compound; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 0.01 mg/kg to about 250 mg/kg, in one embodiment from about 1 mg/kg to about 250 mg/kg, in another embodiment from about 10 mg/kg to about 25 mg/kg, and may be given in a single dose or in two or more divided doses. Variations in the dosage will necessarily occur depending upon the species, weight and condition of the patient being treated and the patient's individual response to the medicament.

In one embodiment, the unit dosage form is about 0.01 to about 1000 mg. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg;

The compound or a pharmaceutically acceptable salt of the compound can be assayed in vitro or in vivo for the desired therapeutic or prophylactic activity prior to use in humans. Animal model systems can be used to demonstrate safety and efficacy.

The present methods for treating or preventing a central nervous system disorder can further comprise administering another therapeutic agent to the animal being administered the compound or a pharmaceutically acceptable salt of the compound. In one embodiment the other therapeutic agent is administered in an effective amount.

Effective amounts of the other therapeutic agents are well known to those skilled in the art. However, it is well within the skilled artisan's purview to determine the other therapeutic agent's optimal effective amount range. The compound or a pharmaceutically acceptable salt of the compound and the other therapeutic agent can act additively or, in one embodiment, synergistically. In one embodiment, where another therapeutic agent is administered to an animal, the effective amount of the compound or a pharmaceutically acceptable salt of the compound is less than its effective amount would be where the other therapeutic agent is not administered. In this case, without being bound by theory, it is believed that the compound or a pharmaceutically acceptable salt of the compound and the other therapeutic agent act synergistically. In some cases, the patient in need of treatment is being treated with one or more other therapeutic agents. In some cases, the patient in need of treatment is being treated with at least two other therapeutic agents. In one embodiment, the other therapeutic agent is selected from the group consisting of one or more anti-depressant agents (e.g., SSRIs, monoamine oxidase inhibitors, norepinephrine reuptake inhibitors, and serotonin and noradrenaline reuptake inhibitors), anti-anxiety agents (e.g., benzodiazepines, serotonin 1A (5-HT_{1A} ) agonists or antagonists (such as 5-HT_{1A} partial agonists), or corticotrophin releasing factor), anti-psychotic agents (e.g., phethiazine, piperainze phenothiazines, butyrophenones, substituted benzamides, thioxanthine, haloperidol, olanzapine, clozapine, risperidone, pimozide, aripiprazol, or ziprasidone), or cognitive enhancers (e.g., acetylcholinesterase or cholinesterase inhibitors, cholinergic receptor agonists, or serotonin receptor antagonists, drugs that modulate the level of soluble Aβ amyloid fibril formation or amyloid plaque burden, or drugs that protect neuronal activity).

In one embodiment, the compound or a pharmaceutically acceptable salt of the compound is administered concurrently with another therapeutic agent.

In one embodiment, a composition comprising an effective amount of the compound or a pharmaceutically acceptable salt of the compound and an effective amount of another therapeutic agent within the same composition can be administered.

In another embodiment, a composition comprising an effective amount of the compound or a pharmaceutically acceptable salt of the compound and a separate composition comprising an effective amount of another therapeutic agent can be concurrently administered. In another embodiment, an effective amount of the compound or a pharmaceutically acceptable salt of the compound is administered prior to or subsequent to administration of an effective amount of another therapeutic agent. In this embodiment, the compound or a pharmaceutically acceptable salt of the compound is administered while the other therapeutic agent exerts its therapeutic effect, or the other therapeutic agent is administered while the compound or a pharmaceutically acceptable salt of the compound exerts its preventative or therapeutic effect for treating or preventing a central nervous system disorder.

Thus, in one embodiment, a composition comprising an effective amount of the compound or a pharmaceutically acceptable salt of the compound of the present invention and a pharmaceutically acceptable carrier is provided. In another embodiment, the composition further comprises a second therapeutic agent. In some embodiments, the second therapeutic agent includes one or more other antidepressants, anti-anxiety agents, anti-psychotic agents or cognitive enhancers. In another embodiment, the pharmaceutically acceptable carrier is suitable for oral administration and the composition comprises an oral dosage form.

In one embodiment, the compounds or pharmaceutically acceptable salts of the compounds described herein are useful as modulators of the activity of a 5-HT_{1A} receptor. For example, in one embodiment, the compounds or pharmaceutically acceptable salts of the compounds described herein bind to a 5-HT_{1A} receptor. In one embodiment, the compounds or pharmaceutically acceptable salts of the compounds described herein are useful as 5-HT_{1A} receptor antagonists. Compounds that modulate the activity of 5-HT_{1A} receptors, such as for example by binding to or antagonizing the receptor, can be readily identified by those skilled in the art using numerous art-recognized methods, including standard pharmacological test procedures such as those described herein. Accordingly, in some embodiments, the compounds and pharmaceutically acceptable salts of the compounds described herein are useful for treating a mammal with a central nervous system disorder that is mediated through the 5-HT_{1A} pathway. Central nervous system disorders include, without limitation, anxiety-related disorders, cognition-related disorders, depression and depression-related disorders, and schizophrenia and other psychotic disorders. Thus, in one embodiment, the compounds and pharmaceutically acceptable salts of the compounds described herein that act as 5-HT_{1A} receptor modulators are useful for treating a mammal with a cognition-related disorder, an anxiety-related disorder, depression or schizophrenia.

Exemplary cognition-related disorders (e.g., cognitive dysfunction) include, without limitation, mild cognitive impairment (MCI), dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorders (e.g., attention deficit hyperactivity disorder (ADHD)) and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the embodiments described herein.

Exemplary anxiety-related disorders include, without limitation, generalized anxiety disorder, attention deficit disorder, attention deficit hyperactivity disorder, obsessive compulsive disorder, substance addiction, withdrawal from drug, alcohol or nicotine addiction, panic disorder, panic attacks, post traumatic stress disorder, premenstrual dysphoric disorder, social anxiety disorder, eating disorders such as anorexia nervosa and bulimia nervosa, vasomotor flushing, and phobias, including social phobia, agoraphobia, and specific phobias. Substance addition includes, without limitation, drug, alcohol or nicotine addiction.

In one embodiment, the compounds or pharmaceutically acceptable salts of the compounds described herein are useful as modulators of serotonin reuptake. For example, in some embodiments, the compounds or pharmaceutically acceptable salts of the compounds described herein can block the reuptake of the brain neurotransmitter serotonin. Accordingly, the compounds or pharmaceutically acceptable salts of the compounds described herein are useful for the treatment or prevention of conditions commonly treated by the administration of serotonin selective reuptake inhibitor (SSRI) antidepressants, such as depression (including but not limited to major depressive disorder, childhood depression and dysthymia), anxiety, panic disorder, post-traumatic stress disorder, premenstrual dysphoric disorder (e.g., pre-menstrual syndrome), attention deficit disorder (with and without hyperactivity), obsessive compulsive disorders (including but not limited to trichotillomania), obsessive compulsive spectrum disorders (including but not limited to autism), social anxiety disorder, generalized anxiety disorder, obesity, eating disorders such as anorexia nervosa, bulimia nervosa, vasomotor flushing, cocaine and alcohol addiction, sexual dysfunction (including but not limited to premature ejaculation), incontinence (including, but not limited to fecal incontinence, urge incontinence, overflow incontinence, passive incontinence, reflex incontinence, stress urinary incontinence urinary exertional incontinence and urinary incontinence), and pain (including, but not limited to migraine, chronic back pain, phantom limb pain, neuropathic pain such as diabetic neuropathy, and post herpetic neuropathy) and related illnesses.

The compounds or pharmaceutically acceptable salts of the compounds described herein are also useful for the treatment or prevention of conditions mediated through the 5-HT_{1A} receptors (e.g., those commonly treated by the administration of 5-HT_{1A} antagonists), such as depression, such as single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder, pediatric depression, child abuse induced depression and postpartum depression; bipolar disorders or manic depression, for example, bipolar I disorder, bipolar II disorder and cyclothymic disorder; conduct disorder; disruptive behavior disorder; disorders of attention and learning such as attention deficit hyperactivity disorder (ADHD) and dyslexia; behavioral disturbances associated with mental retardation, autistic disorder, pervasive development disorder and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia, substance-induced psychotic disorder, shared psychotic disorder, and psychotic disorder due to a general medical condition; delirium, dementia, and amnestic and other cognitive or neurodegenerative disorders, such as Parkinson's disease (PD), Huntington's disease (HD), Alzheimer's disease, senile dementia, dementia of the Alzheimer's type, mild cognitive impairment (MCI), memory disorders, loss of executive function, vascular dementia, and other dementias, for example, due to HIV disease, head trauma, Parkinson's disease, Huntington's disease, Pick's disease, Creutzfeldt Jakob disease, or due to multiple etiologies; cognitive deficits associated with neurological conditions including, for example, Parkinson's disease (PD), Huntington's disease (HD), Alzheimer's disease; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; chemical dependencies and addictions (e.g., dependencies on, or addictions to, alcohol, heroin, cocaine, benzodiazepines, nicotine, or phenobarbitol); behavioral addictions such as an addiction to gambling; and ocular disorders such as glaucoma and ischemic retinopathy; sexual dysfunction associated with drug treatment (e.g., sexual dysfunction associated with SSRI's).

In some embodiments, the compounds and pharmaceutically acceptable salts of the compounds described herein have dual-acting mechanisms. That is, the compounds and pharmaceutically acceptable salts of the compounds have an ability to modulate serotonin reuptake, as well as an ability to modulate 5-HT_{1A} receptors (e.g., through binding or antagonism).

In one embodiment, a method for modulating the activity of a 5-HT_{1A} receptor is provided. The method includes contacting the receptor with one or more compounds or pharmaceutically acceptable salts of the compounds described herein. In one embodiment, a method of binding a 5-HT_{1A} receptor in a patient is provided. The method includes administering to the patient an effective amount of one or more compounds or pharmaceutically acceptable salts of the compounds described herein. In one embodiment, a method of antagonizing a 5-HT_{1A} receptor is provided. The method includes administering an effective amount of one or more compounds or pharmaceutically acceptable salts of the compounds described herein. In some embodiments, the method includes administration to a patient suffering from a 5-HT_{1A} -related disorder. In one embodiment, a method of modulating serotonin reuptake in a patient is provided. The method includes administering an effective amount of one or more compounds or pharmaceutically acceptable salts of the compounds described herein.

In one embodiment, a method for treating depression, comprising administering to a mammal in need thereof a compound or a pharmaceutically acceptable salt of a compound or pharmaceutically acceptable salt of a compound described herein in an amount effective to treat depression is provided. In one embodiment, the method for treating depression includes administering a second therapeutic agent. In some embodiments, the second therapeutic agent is an anti-depressant agent, an anti-anxiety agent, an anti-psychotic agent, or a cognitive enhancer.

In some embodiments, pharmaceutical compositions or medicaments are provided. The pharmaceutical compositions or medicaments include one or more compounds or pharmaceutically acceptable salts of the compounds described herein. In some embodiments, the pharmaceutical compositions also include one or more pharmaceutically acceptable carriers. In some embodiments, the pharmaceutical composition is useful for modulating the activity of a 5-HT_{1A} receptor (e.g., by binding or antagonizing the receptor). In some embodiments, the pharmaceutical composition is useful for modulating serotonin reuptake in a patient. In some embodiments, the pharmaceutical composition is useful for treating a central nervous system disorder. In one embodiment, a pharmaceutical composition for treating depression is provided. The composition includes a compound or a pharmaceutically acceptable salt of a compound described herein.

The compounds and pharmaceutically acceptable salts of compounds described herein can be prepared using a variety of methods starting from commercially available compounds, known compounds, or compounds prepared by known methods. General synthetic routes to many of the compounds described herein are included in the following schemes. It is understood by those skilled in the art that protection and deprotection steps not shown in the Schemes may be required for these syntheses, and that the order of steps may be changed to accommodate functionality in the target molecule. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The following examples illustrate the production of representative compounds of this invention. Compounds of the present invention are prepared in accordance with the following general description and specific examples. Variables used are as defined for Formula I, unless otherwise noted.

As can be seen from Scheme 1, compounds of the general Formula I wherein Q₁ has the Formula II and Q₂ has the Formula III wherein r and t are each 0, s is 1 and X₁ is O or CH₂, can be prepared by combining the appropriately substituted piperidine (free base) of formula (2) with a suitably substituted benzodioxan methanesulfonate (e.g., R is 4-methylphenyl, 4-bromophenyl or 2-nitrophenyl) of formula (1) in a solvent such as dimethyl sulfoxide or an aliphatic alcohol such as n-butanol, at a temperature of 50-150° C. for several hours as illustrated below.

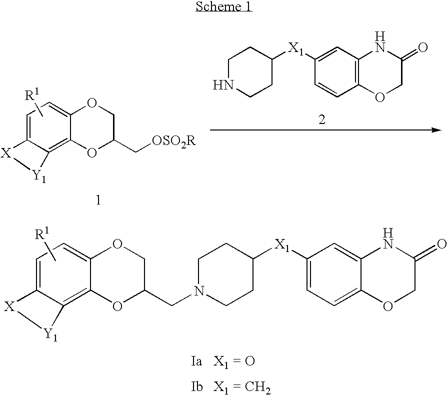

Thus, the present invention provides synthetic methods for making such compounds. The method comprising the step of:

a) reacting a compound of Formula S1:

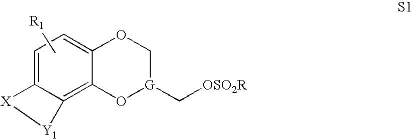

wherein:

G is a carbon atom having a hydrogen atom attached thereto, wherein the carbon atom can have either the R or S configuration;
R is a an appropriately substituted phenyl moiety;
R₁ is hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;
the group X—Y₁ is —N═C(R₂)—C(R₃)═N—, —N═C(R₂)—C(R₄)═CH—, —N═C(R₂)——N═CH—, or —N═C(R₂)—O—;
R₂ and R₃ are, independently, hydrogen, halo, C₁₋₆ alkyl, amino, or mono- or di-alkylamino in which each alkyl group has 1 to 6 carbon atoms;
R₄ is hydrogen or C₁₋₆ alkyl;
   with a compound of Formula S2:

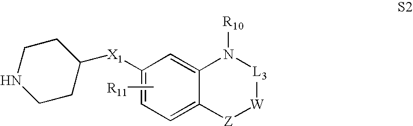

wherein:

R₁₀ is hydrogen, C₁₋₁₂ alkyl or C₄₋₂₀ alkylcycloalkyl, (CH₂)ₓcycloalkyl where x is 4-20, in some embodiments, X is 4-12;

L₃ is —C(═W′)— or CH₂;

W′ is O or S;

W is O, CH₂ or

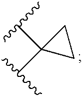

Z is O, CH₂, S or SO₂;

R₁₁ is hydrogen, C₁₋₁₂ alkyl, cyano or halogen;

provided that Z and W are not both O; and

provided that W is not O when Z is S or SO₂;

for a time and under conditions effective to form a compound of Formula S3:

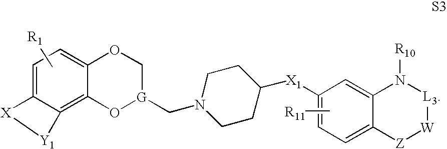

In some embodiments, Z is O, W is CH₂, and L₃ is —C(═W′)— wherein W′ is O.

In some embodiments, X—Y₁ is —N═C(R₂)—C(R₄)═CH—.

In some embodiments, Z is O, W is CH₂, L₃ is —C(═W′)— wherein W′ is O and X—Y₁ is —N═C(R₂)—C(R₄)═CH—.

Alternatively, a salt (hydrochloride, hydrobromide and the like) of the piperidine of formula (2) can be employed by heating in a solvent such as dimethylsulfoxide or an aliphatic alcohol such as n-butanol, in the presence of an organic base such as N,N-diisopropylethylamine or triethylamine, in the presence or absence of 4-(dimethylamino)pyridine.

The substituted piperidine of formula (2a) where X¹ is oxygen, can be prepared as shown in Scheme 2. A suitably protected piperidinol sulfonate (e.g., R is 4-methylphenyl, 4-bromophenyl or 2-nitrophenyl) of formula 3 (wherein P is an amino protecting group, preferably a ter-butyloxycarbonyl group, benzyloxycarbonyl or the like) is reacted with the sodium salt of 6-hydroxy-2H-1,4-benzoxazin-3(4H)-one (5) in an aprotic solvent such as N,N-dimethylformamide at 50-100° C. to provide the protected intermediate of formula (4). Alternatively, the reaction can be carried out in a microwave at 100-150 ° C. for shorter periods of time. Subsequent deprotection of (4) provides the desired substituted piperidine of formula (2a).

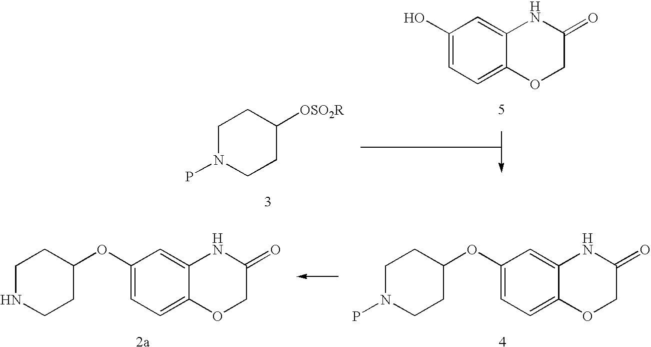

Alternatively, the coupling of 6-hydroxy-2H-1,4-benzoxazin-3(4H)-one (5) and a sulfonate of formula (3a) can be carried out in an aprotic solvent such as N,N-dimethylformamide and in the presence of an inorganic base such as cesium carbonate or the like, at 30-50° C. to provide the aforementioned intermediate of formula (4) as shown in Scheme 2a.

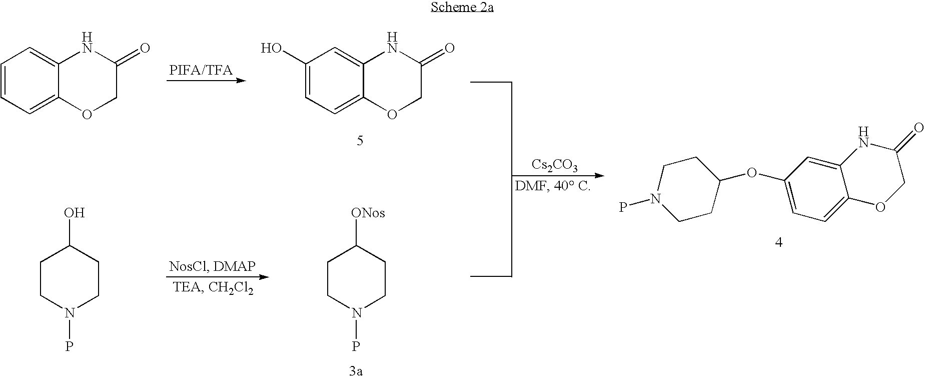

Thus, the invention provides synthetic methods described above, wherein the compound of Formula S2 is prepared by a method comprising the steps of:

(a1) reacting a compound of Formula S4:

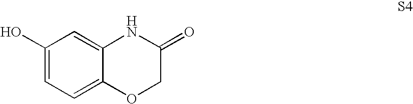

or a salt thereof;
with a compound of Formula S5:

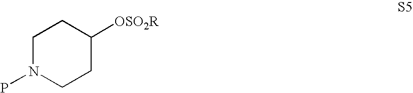

wherein P is an amino protecting group;
for a time and under conditions effective to form a compound of Formula S6:

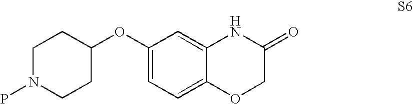

and

(a2) removing the protecting group P to provide the compound of Formula S2.

In some embodiments, the sodium salt of the compound of Formula S4 is reacted with the compound of Formula S5 in an aprotic solvent.

In some embodiments, the reaction of the compound of Formula S4 and the compound of Formula S5 are reacted in an aprotic solvent, in the presence of an inorganic base (e.g., cesium carbonate).

In some embodiments, the reaction of the compound of Formula S4 and the compound of Formula S5 are reacted in an aprotic solvent, in the presence of an inorganic base (e.g., cesium carbonate).

In some embodiments, the compound of Formula S4 is prepared by reacting a compound of Formula S7:

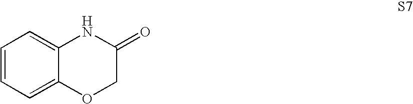

with PIFA in TFA.

In some embodiments, the compound of Formula S5 is prepared by reacting a compound of Formula S8:

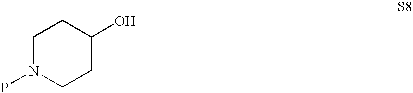

with tosyl chloride or nosyl chloride.

Alternatively, the protected piperidine of formula (4) of Scheme 2 can be prepared from 6-bromo-2H-1,4-benzoxazin-3(4H)-one (6,cf. Mazharuddin M. et al., Indian J. Chem. 7, 658(1969); J. Chem. Res., Synopses, 681, M 1120(2003); J. Nat. Products 63, 480 (2000)) and the protected piperidinol of formula (7) by a palladium catalyzed coupling (cf. Torraca K. E. J. Am. Chem. Soc. 123, 10770 (2001)), as shown in Scheme 3.

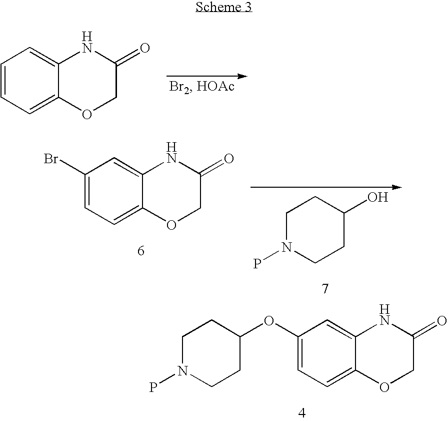

Thus, the invention provides synthetic methods as described above, wherein the compound of Formula S2 is prepared by a method comprising the steps of:

(b1) reacting a compound of Formula S7:

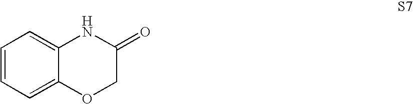

with Br₂ in acetic acid to form a compound of Formula S9:

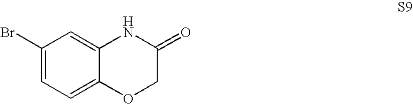

and

(b2) reacting the compound of S9 with a compound of Formula S8:

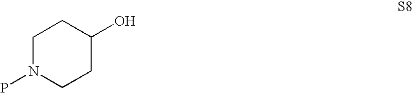

to form the compound of Formula S2.

Another preferred process for the preparation of 6-bromo-2H-1,4-benzoxazin-3(4H)-one (6) consists of a two-step process illustrated in Scheme 3a. Thus, 4-bromo-2-nitrophenol is alkylated with methyl bromoacetate in mixture of acetone and N,N-dimethylformamide and in the presence of sodium iodide and an inorganic base such as cesium carbonate at or below room temperature. The intermediate ether is then reduced and simulateneously cyclized to the desired (6) by treatment with iron powder in a mixture of ethanol and acetic acid at or below room temperature.

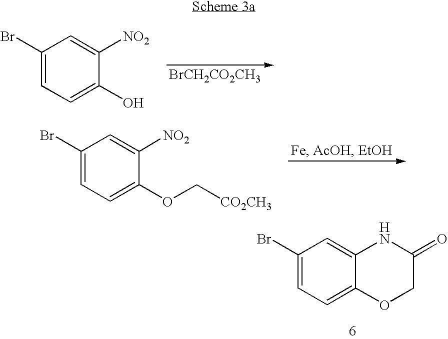

Another preferred process for the preparation of the protected piperidine of formula (4) of Scheme 2 consists of a Mitsunobu coupling of 6-hydroxy-2H-1,4-benzoxazin-3(4H)-one of formula (5) with the protected piperidinol of formula (7) in an aprotic solvent such as teterahydrofuran, as shown in Scheme 4 (cf. Tetrahedron Lett. 39, 8751 (1998); Bioorg. Med. Chem. Lett. 13, 855 (2005)).

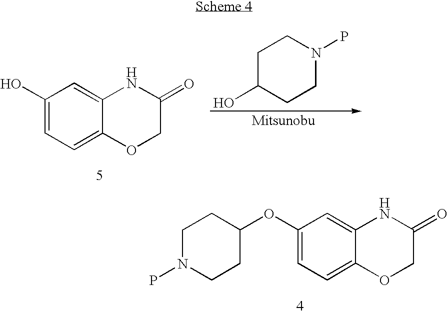

Thus, the invention provides synthetic methods as described above, wherein the compound of Formula S4 is prepared by a method comprising the steps of:

(c1) reacting a compound of Formula S4:

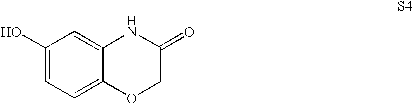

with a compound of Formula S8:

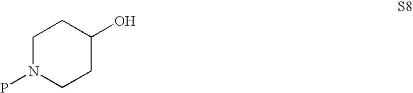

for a time and under conditions effective to form the compound of Formula S6;

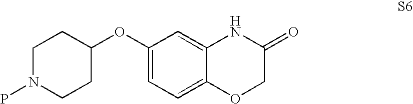

and

(c2) removing the protecting group P to provide the compound of Formula S2.

In some embodiments, the reaction of the compound of Formula S4 and S8 is performed under Mitsunobu conditions.

An alternate approach to the target compound (4) relies on the Williamson-type coupling of the phenol (5) and the piperidinol (7) as shown in Scheme 5 (cf. Aspinall H. C. et al. Tetr. Lett. 38, 4679 (1997)).

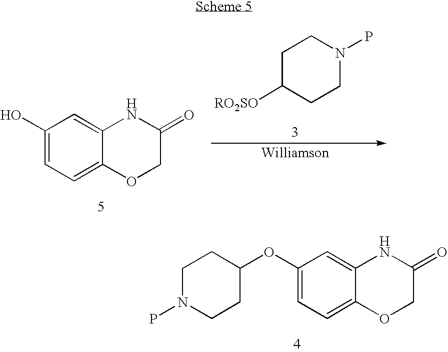

Thus, the invention provides synthetic methods as described above, wherein the reacting of the compound of Formula S4 and S5 is performed under Williamson conditions.

Alternatively, the desired compounds of formula Ia can be conveniently prepared via a Mitsunobu coupling of 6-hydroxy-2H-1,4-benzoxazin-3(4H)-one (5) and an appropriately substituted piperidin-4-ol of formula (69) in an aprotic solvent such as tetrahydrofuran, as shown in Scheme 5a below.

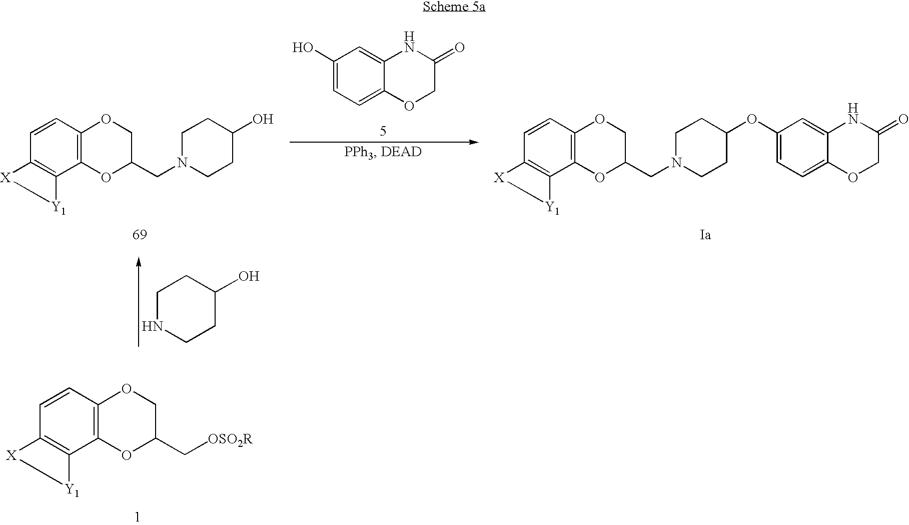

Thus, the invention provides synthetic methods for preparing compounds of formula I, comprising the step of:

a) reacting a compound of Formula S10:

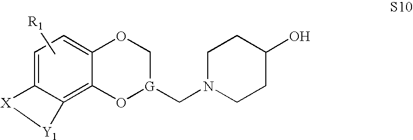

wherein:

G is a carbon atom having a hydrogen atom attached thereto, wherein the carbon atom can have either the R or S configuration;
R₁ is hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;
the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, halo, C₁₋₆ alkyl, amino, or mono- or di-alkylamino in which each alkyl group has 1 to 6 carbon atoms;
R₄ is hydrogen or C₁₋₆ alkyl; with a compound of Formula S11:

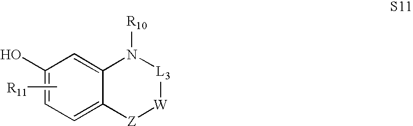

wherein:
R₁₀ is hydrogen, C₁₋₁₂ alkyl or C₄₋₂₀ alkylcycloalkyl;
L₃ is —C(═W′)— or CH₂;
W′ is O or S;
W is O, CH₂ or

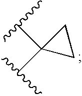

Z is O, CH₂, S or SO₂;
R₁₁ is hydrogen, C₁₋₁₂alkyl cyano or halogen;
provided that Z and W are not both O; and
provided that W is not O when Z is S or SO₂,
   for a time and under conditions effective to form a compound of Formula S12:

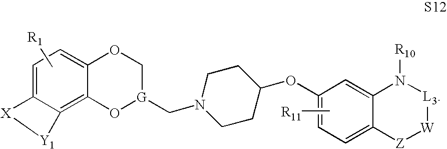

In some embodiments, Z is O, W is CH₂, and L₃ is —C(═W′)— wherein W′ is O.

In some embodiments, X—Y₁ is —N═C(R₂)—C(R₄)═CH—.

In some embodiments, Z is O, W is CH₂, L₃ is —C(═W′)— wherein W′ is O, and X—Y₁ is —N═C(R₂)—C(R₄)═CH—.

In some embodiments, the compound of Formula S10 is prepared by a method comprising the step of:

(d1) reacting a compound of Formula S1:

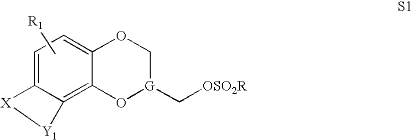

wherein R is a protecting group;

with 4-hydroxypiperidine, for a time and under conditions effective to form the compound of Formula S10.

The 6-hydroxy-2H-1,4-benzoxazin-3(4H)-one of formula (5) can be prepared starting from 6-acetyl-2H-1,4-benzoxazin-3(4H)-one (8) as shown in Scheme 6, according to the procedure described in WO 2004/089915 A1.

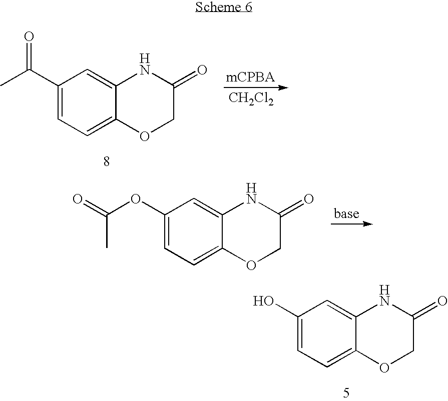

Alternatively, the compound of formula (5) can be prepared from 2H-1,4-benzoxazin-3(4H)-one according to the procedure of Itoh et al. (J. Org. Chem. 67, 7424 (2002)) as shown in Scheme 7.

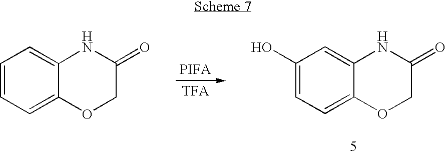

The substituted piperidine of formula 2b where, in terms of Formula I, Q₂ has the Formula III wherein X₁ is CH₂, v is 1, s is 1, r and t are each 0, and P¹ is an amino protecting group (preferably a benzyloxycarbonyl or benzyl group), can be prepared starting from 4-methoxy-5-nitro toluene as shown in Scheme 8.

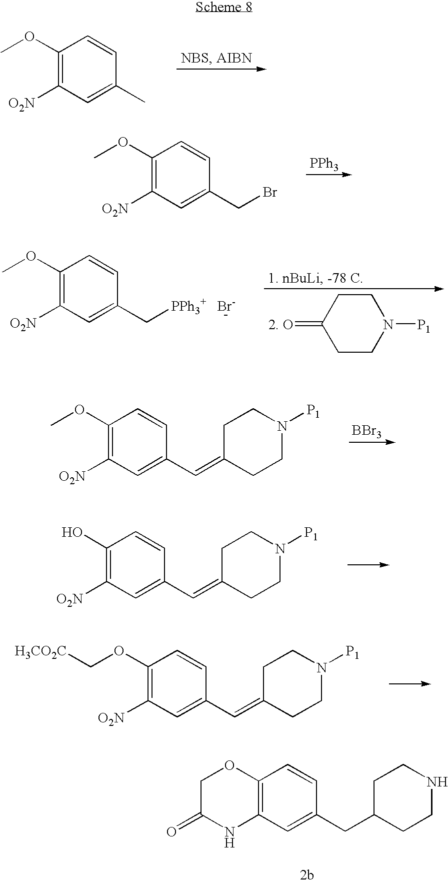

Alternatively, in the procedure of Scheme 8 the phosphonium salt can be replaced by the appropriate phosphonate in a Wadsworth-Emmons type modification of the Wittig reaction (cf. J. March, Advanced Organic Chemistry, 3^{rd} Edn, J. Wiley and Sons, NY 1985, p 848), as shown in Scheme 8a for the preparation of the substituted piperidine of formulas 2h and 2i where, in terms of Formula I, Q₂ has the Formula III wherein X₁ is CH₂, v is 2, s is 1, r and t are each 0, and P² is an amino protecting group (preferably tert-butyloxycarbonyl), and R¹⁰ is alkyl or hydrogen, respectively.

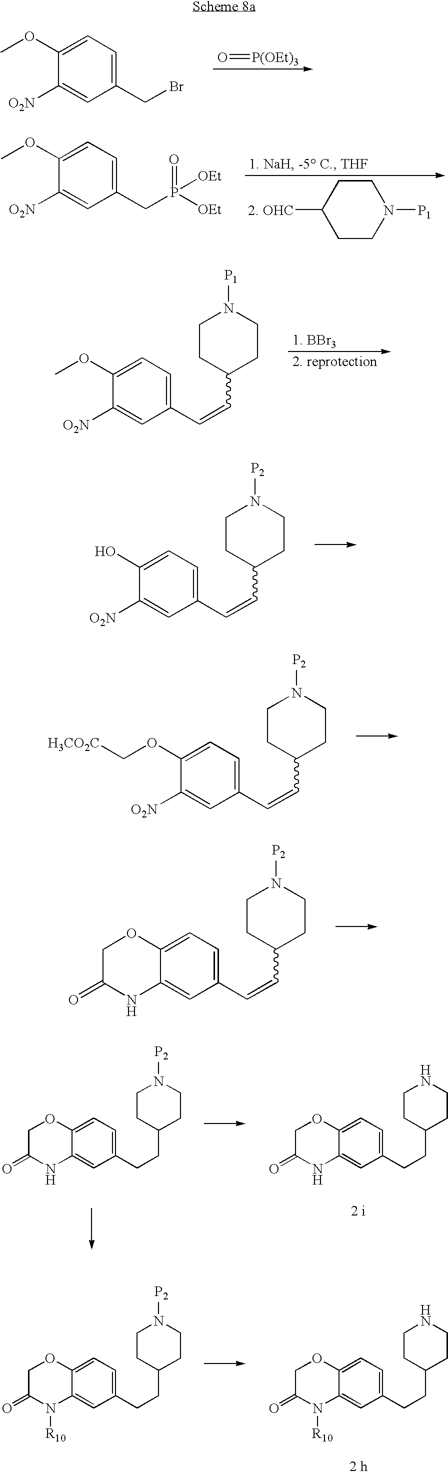

In a similar manner one can prepare as shown in Scheme 8b the substituted piperidines of formula 2j and 2k where, in terms of Formula I, Q₂ has the Formula III wherein X₁ is CH₂, v is 1, s is 1, r and t are each 0, and P₂ is as defined hereinbefore, and R¹⁰ is alkyl or hydrogen respectively.

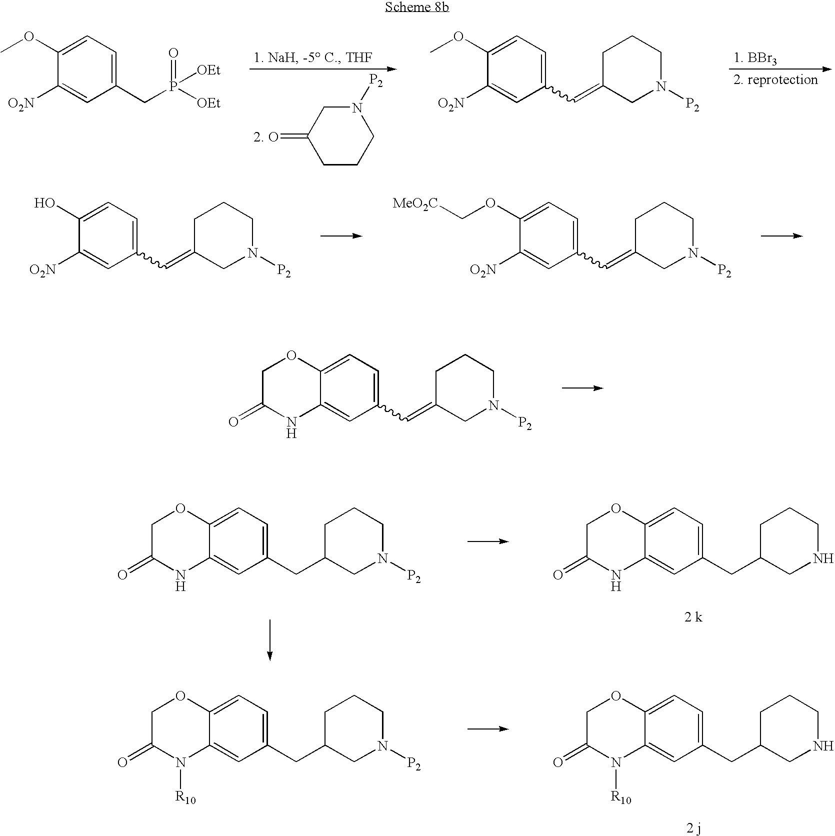

Alternatively, the substituted piperidine of formula (2b) can be prepared as shown in Scheme 9 by acylation of 2H-1,4-benzoxazin-3(4H)-one (cf. Indian J. Chem. 28B, 882 (1989); Indian J. Chem. 27B, 871 (1988); Heterocycl. Comm. 6, 477 (2000); Heterocyclic Comm. 9, 51 (2003)) to give the intermediate ketone of formula (10) which can in turn, be reduced to (11) which can be deprotected to give (2b). P₂ is an amino protecting group chosen as to be compatible with the synthetic sequence of Scheme 9.

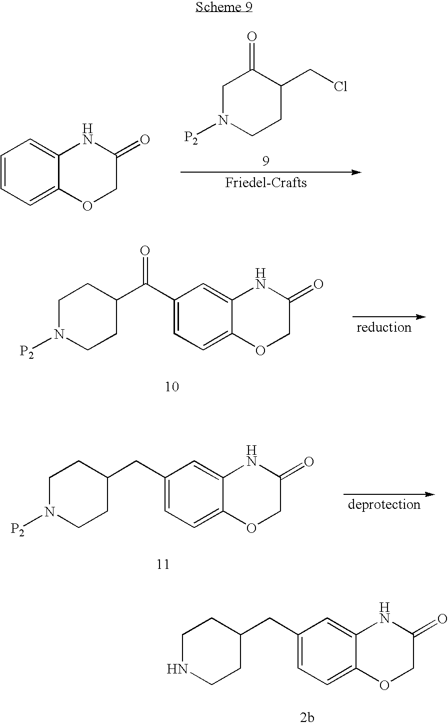

An alternative process for the preparation of a compound of formula (2c) where, in terms of Formula I, Q₂ has the Formula III wherein r and s are each 0, t is 1, and R₇ is alkyl from the intermediate ketone (10) of Scheme 9 is shown in Scheme 10.

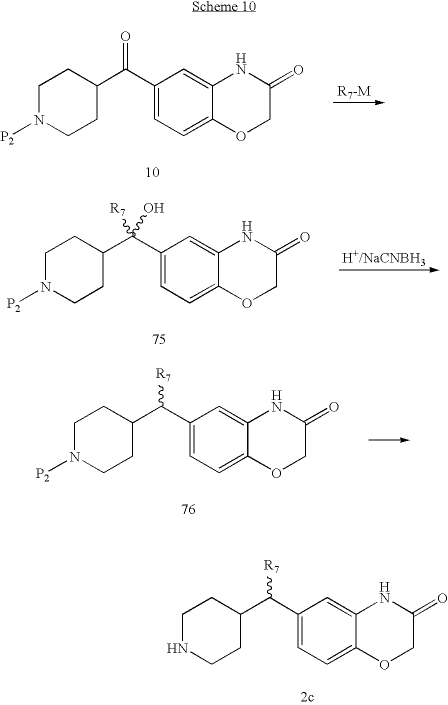

Compounds of Formula I, wherein Q₁ has the Formula II, k is 0, n is 2, each R₆ is hydrogen, L is CH, X₁ is O, W is CH₂, Z is O, R₁₀ is alkyl, Y is CH and W′ is O (cf, Example 5), can be prepared according to Scheme 1 by replacing the intermediate of formula (2) wherein X₁ is O with the 4-alkyl-6-(piperidin-4-yloxy)-2H-1,4-benzoxazin-3(4H)-one of formula (70) as shown in Scheme 10a.

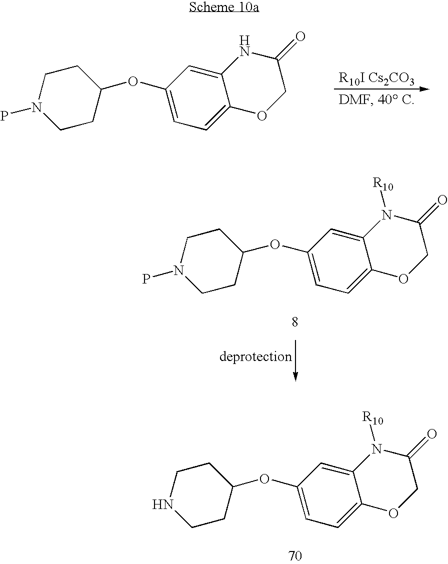

Compounds of Formula I wherein Q₁ is —N(R₈)—, and Q₂ has the Formula III wherein r is 4, s and t are each 0, X₁ is O, each R₆ₐ and R_{6b} is hydrogen, W is CH₂, Z is O, Y is CH, W′ is O, and R₁₀ is hydrogen, can be prepared by coupling the amine of formula (72) with the 6-(4-bromobutoxy)-2H-1,4-benzoxazin-3(4H)-one of formula (71) as shown in Scheme 10b.

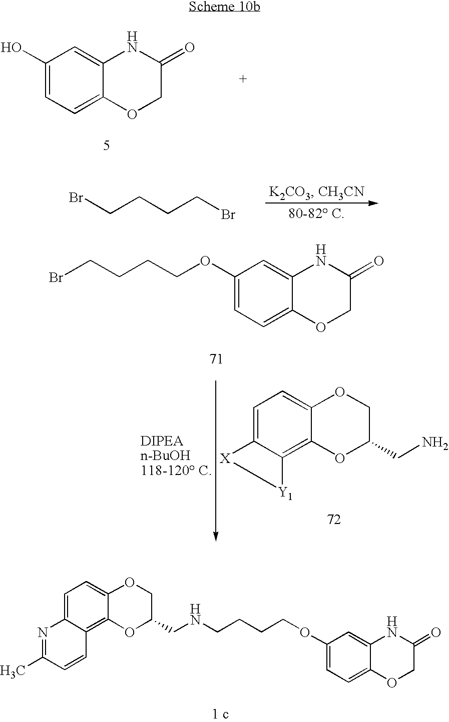

Compounds of Formula I wherein Q₁ is —N(R₈)—, and Q₂ has the Formula III wherein r is 4, s and t are each 0, X₁ is O, each R₆ₐ and R_{6b} are hydrogen, W is CH₂, Z is O, Y is CH, W′ is O, and R₁₀ is hydrogen, can be prepared according to Scheme 10b but replacing the bromide of formula formula (71) with the corresponding 6-(4-bromopropoxy)-2H-1,4-benzoxazin-3(4H)-one.

Compounds of Formula I wherein Q₁ has the Formula II, k is 0, n is 1, R₆ is hydrogen, L is CH or CH₂, rand t are each 0, X₁ is O, W is CH₂, Z is O, Y is CH, W′ is O, and R₁₀ is hydrogen (cf, Example 8), can be prepared according to Scheme 1 by replacing the intermediate of formula (2) wherein X¹ is O with the 6-[(3S)-pyrrolidin-3-yloxy]-2H,1,4-benzoxazin-3(4H)-one of formula (73) as shown in Scheme 10c.

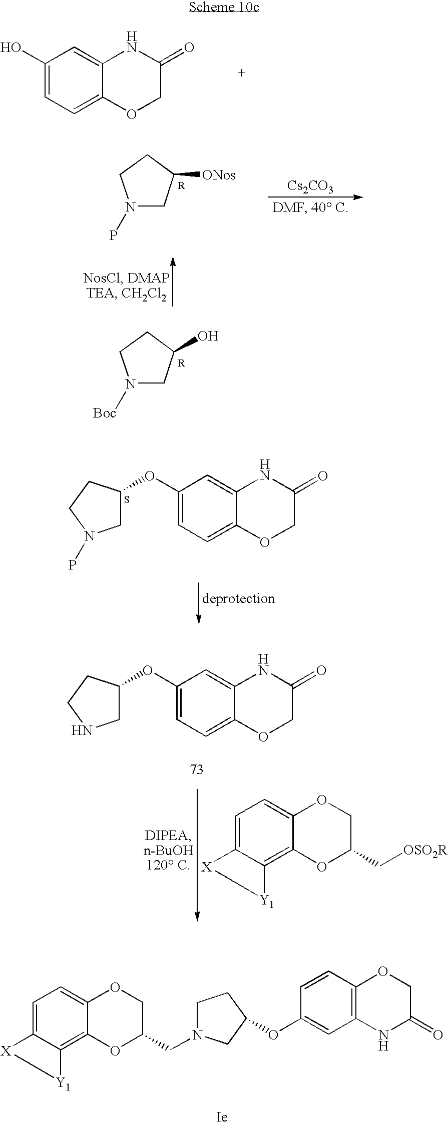

By replacing the amine (73) with its enantiomer (74) in Scheme 10c the compound of Formulas If can be prepared as shown in Scheme 11.

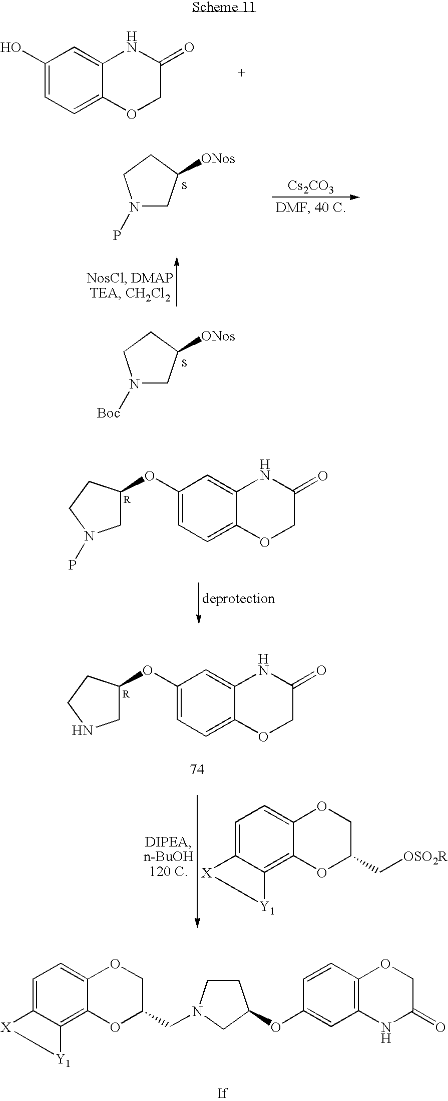

The compounds of formula I wherein Q₁ has formula II wherein J is NH and k is 1, L₁ is CH, L₂ is N, R₆ is hydrogen and n is 2, and Q₂ has formula III wherein r,s and t are each 0 can be conveniently prepared as shown in Scheme 12.

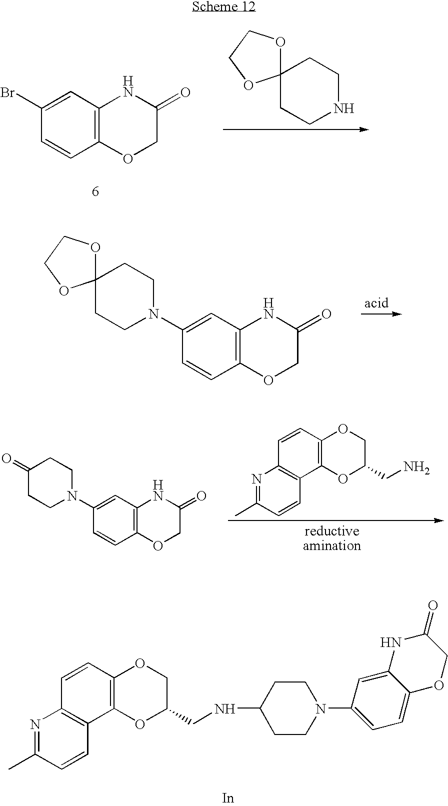

The 2,3-dihydro-1,4-dioxino[2,3-f]quinolin-2-ylmethyltosylates in which R₂ is hydrogen (R is 4-methylphenyl) appropriate to the chemistry in Scheme 1 can be prepared as illustrated in Scheme 13 below. Specifically, the appropriately substituted nitroguaiacol (19) is alkylated with allyl bromide in the presence of a suitable base such as sodium hydride to produce (20) and then demethylated by a reagent such as sodium hydroxide. The resulting 4-nitro-2-allyloxyphenol (21) is then alkylated with glycidyl tosylate or an epihalohydrin in the presence of a base such as sodium hydride to produce (22) and heated in a high boiling solvent such as mesitylene or xylene to effect both rearrangement of the allyl group and cyclization to the dioxan ring. The resulting primary alcohol (23) is converted to the tosylate by reaction with p-toluenesulfonyl chloride in the presence of a tertiary amine or alternatively to a halide by reaction with carbon tetrabromide or carbon tetrachloride in combination with triphenylphosphine. The allyl side chain is then isomerized by treatment with catalytic bis-acetonitrile palladium (II) chloride in refluxing methylene chloride or benzene to produce (24). Allylic oxidation with selenium dioxide in refluxing dioxane/water gives the o-nitrocinnamaldehyde, which upon reduction with iron in acetic acid cyclizes to the 2,3-dihydro-1,4-dioxino[2,3-f]quinoline-2-methyl-tosylate (25).

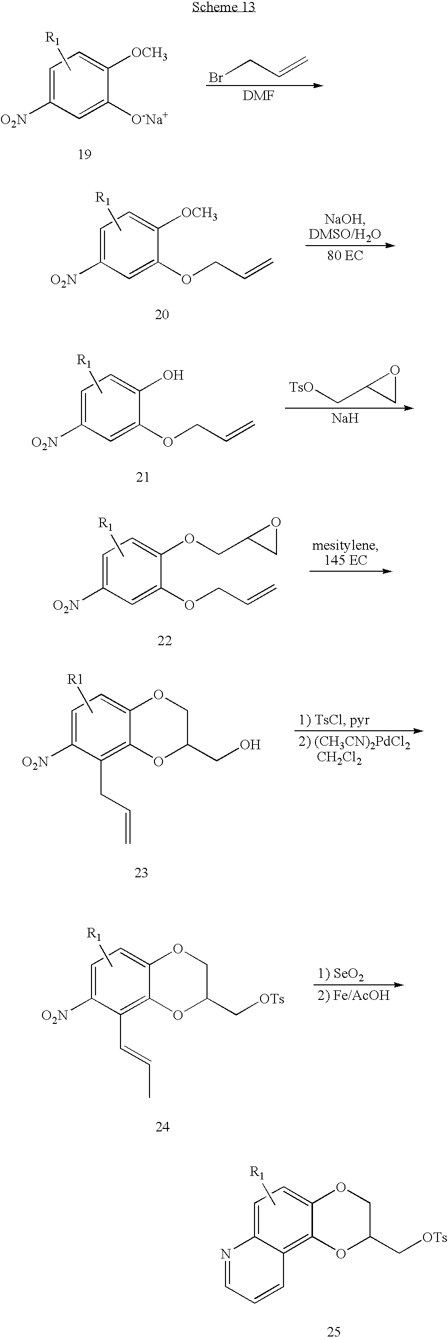

The 2,3-dihydro-1,4-dioxino[2,3-f]quinolin-2-ylmethyltosylates in which R₂ is alkyl may be prepared from the nitro olefin described above in the manner described in Scheme 14. The rearranged olefin (24) is treated sequentially with ozone and a tertiary amine or with osmium tetroxide and sodium periodate to give the o-nitrobenzaldehyde (26). Condensation with the appropriate triphenylphosphoranylidene ketone under Wittig conditions gives the o-nitrostyryl ketone (27), which upon reduction by iron in acetic acid, cyclizes to the corresponding 2,3-dihydro-1,4-dioxino[2,3-f]-quinoline-2-methyltosylate (28). Replacement of the tosylate with the appropriately substituted amines gives the title compounds of the invention.

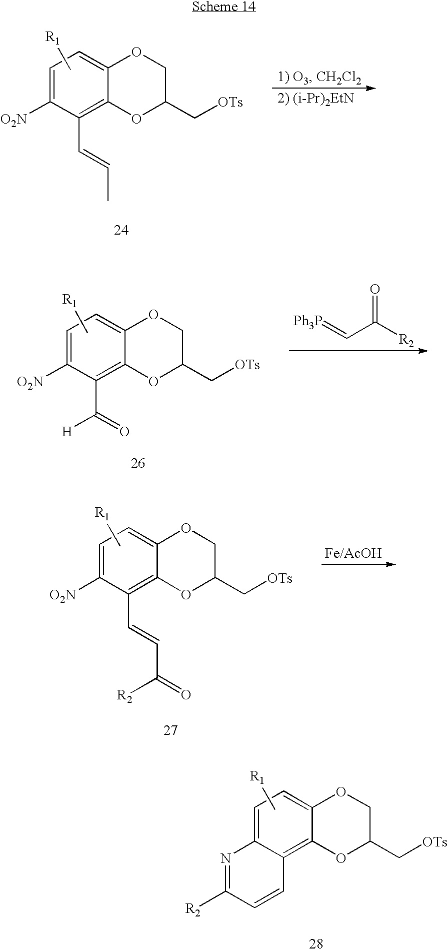

Substitution of trimethyl phosphonoacetate for the triphenylphosphoranylidene ketone in the Wittig procedure above, followed by reduction of the nitro group with tin (II) chloride and cyclization in acid gives the compounds of the invention in which R₂ is hydroxy. Alkylation of this hydroxy derivative by a suitable alkyl halide or tosylate in the presence of base gives the compounds of the invention in which R₂ is alkoxy. Treatment of the hydroxy derivative with an inorganic acid chloride such as phosphoryl chloride or bromide gives the compounds of the invention in which R₂ is halo. Substitution of diethyl cyanomethylphosphonate for the triphenyl-phosphoranylidene ketone in the Wittig procedure above, followed by reduction of the nitro group with tin (II) chloride and cyclization in acid gives the compounds of the invention in which R₂ is amino.

The o-nitrobenzaldehyde (26) used in the Wittig chemistry described in Scheme 14 may be alternatively prepared as shown in Scheme 15. The appropriate mono-allylated catechol (29) is elaborated with glycidyl tosylate as described above to produce (30) and rearranged in refluxing mesitylene. Cyclization to the benzodioxan methanol is effected by treatment with sodium bicarbonate in ethanol and the alcohol (31) is converted to the tosylate. After rearrangement of the double bond by treatment with catalytic bis-acetonitrile palladium chloride in refluxing methylene chloride to produce (32) and cleavage with ozone or osmium tetroxide/sodium periodate as described above, the resulting aldehyde (33) is regioselectively nitrated with a combination of nitric acid and tin (IV) chloride to produce (26).

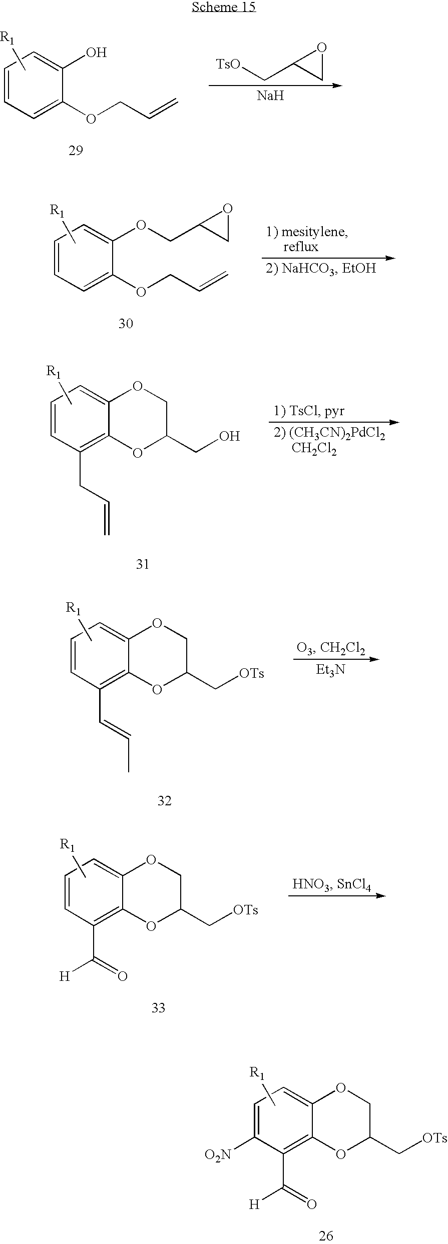

Compounds of the invention in which R₁ is attached to position 6 of the 2,3-dihydro-1,4-dioxino[2,3-f]quinoline may be alternatively prepared by a variation of the Skraup quinoline synthesis according to Scheme 16. The appropriately substituted benzodioxan methyltosylate (34) is nitrated under standard conditions with nitric acid in a solvent such as dichloroethane and the resulting nitro compound (35) reduced by treatment with hydrogen in the presence of a catalyst such as platinum on sulfide carbon. Treatment of the resulting aniline (36) with acrolein in the presence of hydrogen chloride and an oxidant such as p-chloranil or naphthoquinone gives the corresponding 2,3-dihydro-1,4-dioxino[2,3-f]quinoline (37). Replacement of the tosylate with the appropriately substituted amine as above gives the title compounds of the invention.

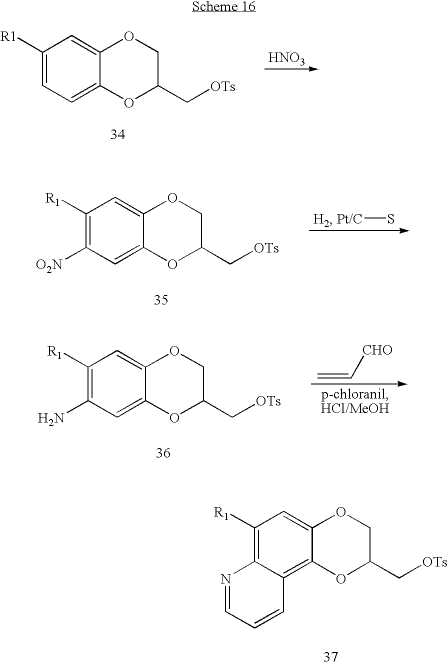

The 2,3-dihydro-1,4-dioxino[2,3-f]quinazolin-2-ylmethylamines of the invention are prepared as illustrated below (Scheme 17). The o-nitrobenzaldehyde (26) described above is converted to the oxime (38) by treatment with hydroxylamine hydrochloride in the presence of a suitable base such as sodium acetate and the nitro group reduced to the amine by hydrogenation over palladium on carbon. Cyclization to the quinazoline N-oxide is effected by treatment at reflux with the appropriate ortho ester according to the method of Ostrowski (Heterocycles, vol. 43, No. 2, p. 389, 1996). The quinazoline N-oxide may be reduced to the quinazoline (39) by a suitable reducing agent such as hydrogen over Raney-nickel. Alternatively, an extended period of reflux in the ortho ester gives the reduced quinazoline directly via a disproportionation reaction and the 2,3-dihydro-1,4-dioxino[2,3-f]quinazoline-2-methyltosylate may be isolated by column chromatography. Replacement of the tosylate or halide with the appropriately substituted amine in some high boiling solvent such as dimethyl sulfoxide gives the title compounds of the invention.

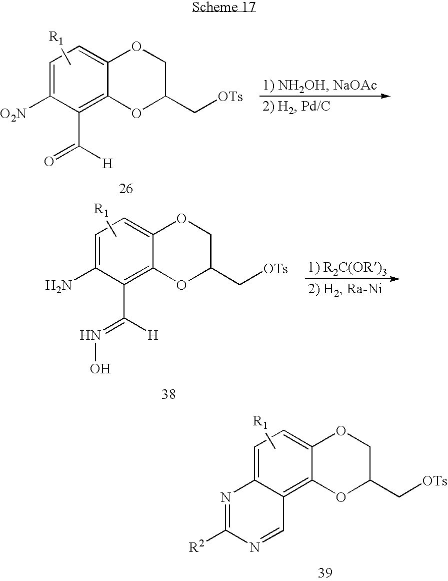

The 2,3-dihydro-1,4-dioxino[2,3-f]quinazolin-2-ylmethylamines of the invention may be alternatively prepared from the rearranged olefin described above by the method outlined in Scheme 18 below. The nitro olefin (24) is first reduced to the aniline by treatment with a suitable reducing agent such as stannous chloride dihydrate in refuxing ethyl acetate and the resulting amine acylated with the appropriate acyl halide or anhydride. The olefin (40) is then converted to the aldehyde (41) by cleavage with catalytic osmium tetroxide in the presence of excess sodium periodate. Cyclization directly to the 2,3-dihydro-1,4-dioxino[2,3-f]quinazoline-2-methyltosylate (39) or halide is effected by treatment of the amido aldehyde (41) with ammonia and replacement of the tosylate or halide with the appropriately substituted amine in some high boiling solvent such as dimethyl sulfoxide as described above gives the title compounds of the invention.

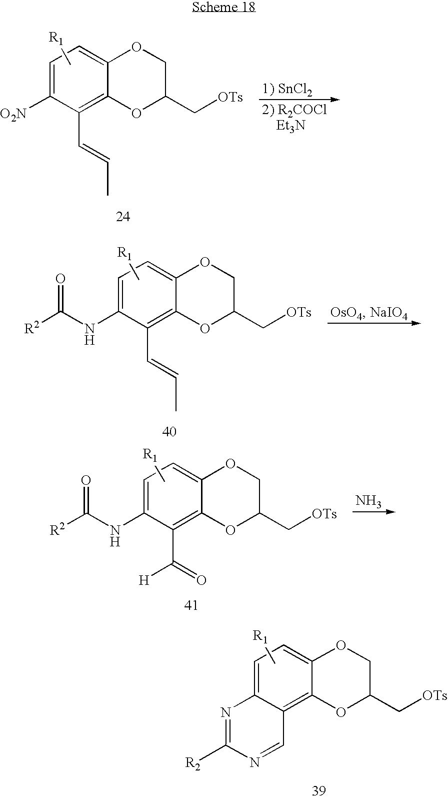

The 2,3-dihydro-1,4-dioxino[2,3-f]quinoxalin-2-ylmethylamines of the invention are prepared as illustrated in Scheme 19 below. The o-nitrobenzaldehyde (26) described above is oxidized to the o-nitrobenzoic acid (42) by a suitable oxidant such as chromium trioxide (Jones' oxidation) or sodium chlorite and the acid converted to the o-nitroaniline (43) with diphenylphosphoryl azide (DPPA) in the presence of a tertiary base such as diisopropylethylamine. Reduction of the resulting nitroaniline to the diamine (44) with hydrogen and palladium on carbon and cyclization by treatment with the appropriate dicarbonyl compound (for example, glyoxal, 2,3-butanedione, 3,4-hexanedione) gives the 2,3-dihydro-1,4-dioxino[2,3-f]quinoxaline-2-methyltosylate (45). Replacement of the tosylate with the appropriately substituted amine in some high boiling solvent such as dimethyl sulfoxide gives the title compounds of the invention.

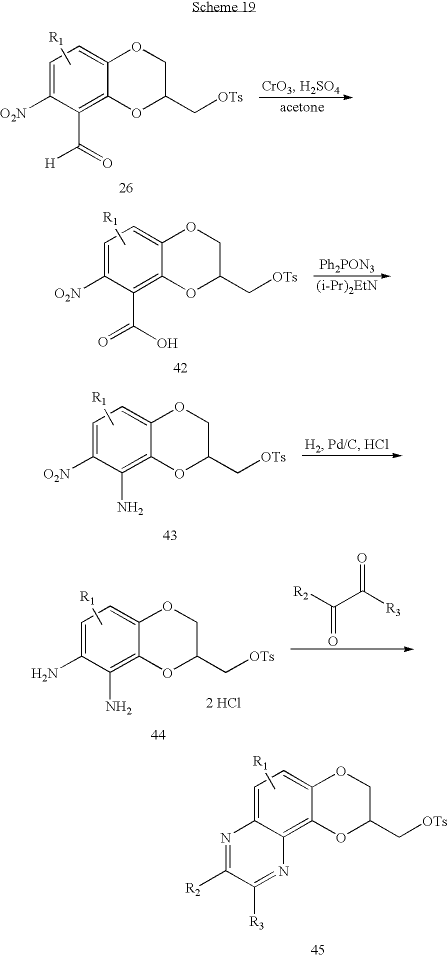

The 7,8-dihydro[1,4]dioxino[2,3-g][1,3]benzoxazol-8-ylmethylamines of the invention are prepared as illustrated in Scheme 20 below. The o-amidobenzaldehyde (41) described in Scheme 18 is converted to the phenol (46) by treatment with meta-chloroperoxybenzoic acid in a Baeyer-Villager reaction and cyclization to the 7,8-dihydro[1,4]dioxino[2,3-g][1,3]benzoxazole (47) is effected by treatment at reflux with an appropriate dehydrating agent such as an ortho ester or an acid catalyst such as p-toluenesulfonic acid. Replacement of the tosylate with the appropriately substituted amine in some high boiling solvent such as dimethyl sulfoxide gives the title compounds of the invention.

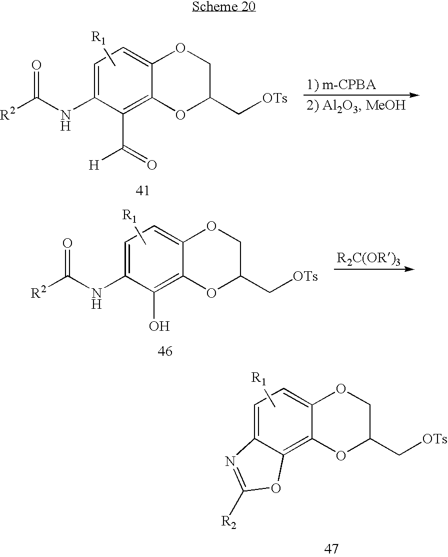

Alternatively (Scheme 21), the nitro olefin (24) may be reduced with tin (II) chloride as described in Scheme 18 above and protected with a suitable protecting group such as carbobenzoxy (Cbz) before the olefin is cleaved to the aldehyde (49) by treatment with osmium tetroxide/sodium periodate and the aldehyde converted to a phenol (50) by the Baeyer-Villager procedure. Deprotection by treatment with hydrogen over palladium on carbon gives the o-aminophenol, (51) which is cyclized to the 7,8-dihydro[1,4]dioxino[2,3-g][1,3]benzoxazole (47) by treatment with the appropriate ortho ester, carboxylic acid or anhydride. Treatment of the o-aminophenol with cyanogen bromide or chloride or a suitably substituted carbamoyl chloride leads to compounds of the invention in which R² is amino. Treatment of the o-aminophenol with carbonyl diimidazole gives the oxazolone which leads to compounds of the invention in which R² is halo via treatment with an inorganic anhydride such as phosphoryl chloride or bromide. Replacement of the tosylate with the appropriately substituted amine as above gives the title compounds of the invention.

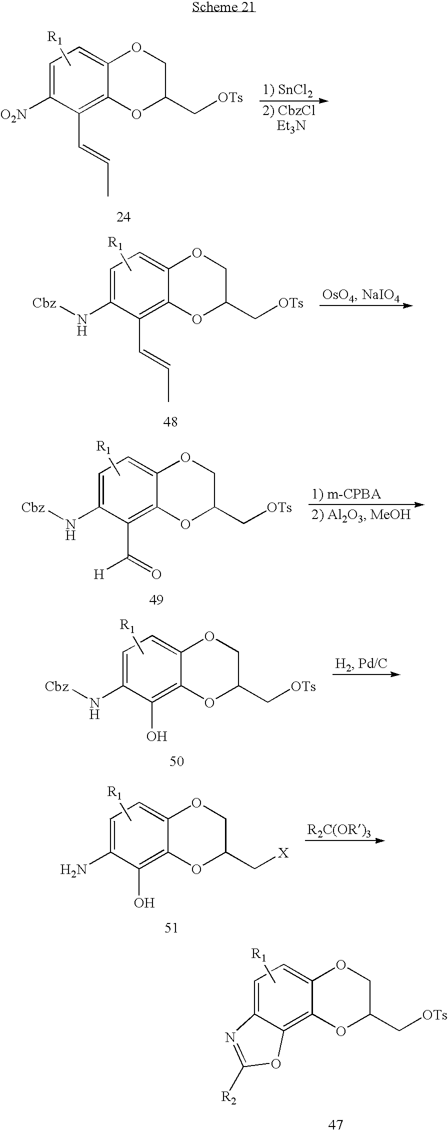

Compounds of the invention in which R₁ is hydrogen and R₂ is alkyl are most conveniently prepared according to Scheme 22 below. The appropriate 2′,3′,4′-trihydroxyacylphenone (52) is regioselectively alkylated with glycidyl tosylate or an epihalohydrin in the presence of a base such as sodium carbonate to give the corresponding 7-acyl-8-hydroxybenzodioxan-2-methanol (53). Following conversion of the ketone to the oxime (54) by reaction with hydroxylamine hydrochloride and sodium acetate, cyclization to the oxazole (55) is effected by treatment with phosphoryl chloride in the appropriate dimethylalkanoic acid amide. The resulting 7,8-di hydro-1,6,9-trioxa-3-aza-cyclopenta[a]naphthalene-8-methanol is converted to the tosylate (56) by treatment with p-toluenesulfonyl chloride in pyridine and combined with the appropriate amine as described above to give the title compounds of the invention.

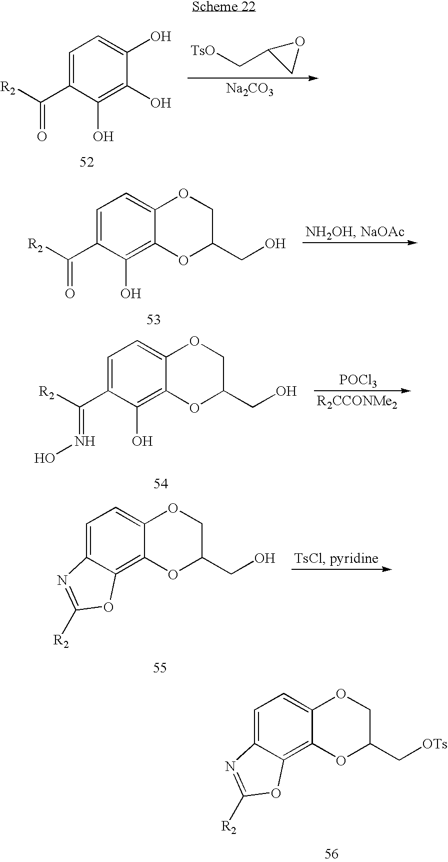

In yet another method, compounds of the present invention may be prepared in accordance with Scheme 23. The synthesis of compound I is comprised of steps that begin with halogenation of 57 where R′ is alkyl of 1-6 carbon atoms, with reagents such as N-halosuccinimide in acetonitrile to give 58 (where Hal is halogen such as Br, Cl or I). Deprotection of 58 with Lewis acids such as boron tribromide, boron trichloride, aluminum trichloride, ferric chloride, or trimethylsilyl iodide in a suitable solvent such as methylene chloride, or with strong protic acids such as HBr and HCl gives the salt 59. Free base 59 may be obtained by neutralization with an Amberlyst A-21 resin slurry in polar solvents such as ethanol or methanol. Alkylation of 59, either as the free base or as the salt, with benzyl or substituted benzyl protected glycidyl ethers

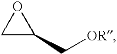

where R″ is benzyl, substituted benzyl such as 4-bromobenzyl, 3,4-dimethoxybenzyl, 2- or 4-nitrobenzyl, or 4-methoxybenzyl) in suitable polar solvents such as dimethyl sulfoxide, dimethyl formamide, or dimethyl acetamide in the presence of bases such as sodium carbonate, potassium carbonate, or triethylamine gives 60. The compound 60 is then cyclized using palladium catalysts such as tris(dibenzylideneacetone)dipalladium, tetrakis(triphenylphosphine)palladium, or palladium acetate with ligands from the group consisting of (±) BINAP and separate enantiomers thereof, (±) Tol-BINAP and separate enantiomers thereof; 1-1′-bis(diphenylphosphino)ferrocene, 1,3-bis(diphenylphosphino)propane, and 1,2 bis(diphenyl-phosphino)ethane in the presence of bases such as NaH, LiH, KH, potassium carbonate, sodium carbonate, titanium carbonate, cesium carbonate, potassium t-butoxide or potassium phosphate tribasic in a suitable solvent such as toluene, or alternatively, with copper catalyst such as copper iodide in the presence of bases such NaH, LiH, KH in a suitable solvent such as toluene to afford 61. Deprotection of 61 with Lewis acids such as boron tribromide, boron trichloride, aluminum trichloride, ferric chloride, trimethylsilyl iodide in a suitable solvent such as methylene chloride, or with strong protic acids such as HBr and HCl or under reductive cleavage conditions using Pd catalyst and hydrogen transfer reagents such as hydrogen, cyclohexene, methyl cyclohexene, or ammonium formate gives 62. The hydroxyl moiety of 62 can be activated with an aryl- or alkyl-sulfonyl chloride such as p-toluenesulfonyl chloride, methanesulfonyl chloride, 2-, 3- or 4-nitrobenzenesulfonyl chloride, or 2- or 4-bromobenzenesulfonyl chloride in the presence of bases such as triethylamine or pyridine in suitable solvents such as methylene chloride, THF, or toluene to afford 63 where R′″ is sulfonate such as p-toluenesulfonate, methanesulfonate, 2-, 3-, or 4-nitrobenzenesulfonate, or 2- or 4-bromobenzenesulfonate. The final coupling of 63 with piperidines appropriate to the invention, in the presence of bases such as diisopropyl ethylamine, potassium carbonate, or sodium carbonate in polar solvents such as THF, dioxane, DMSO, DMF, or DMA and mixtures thereof affords compounds of Formula I.

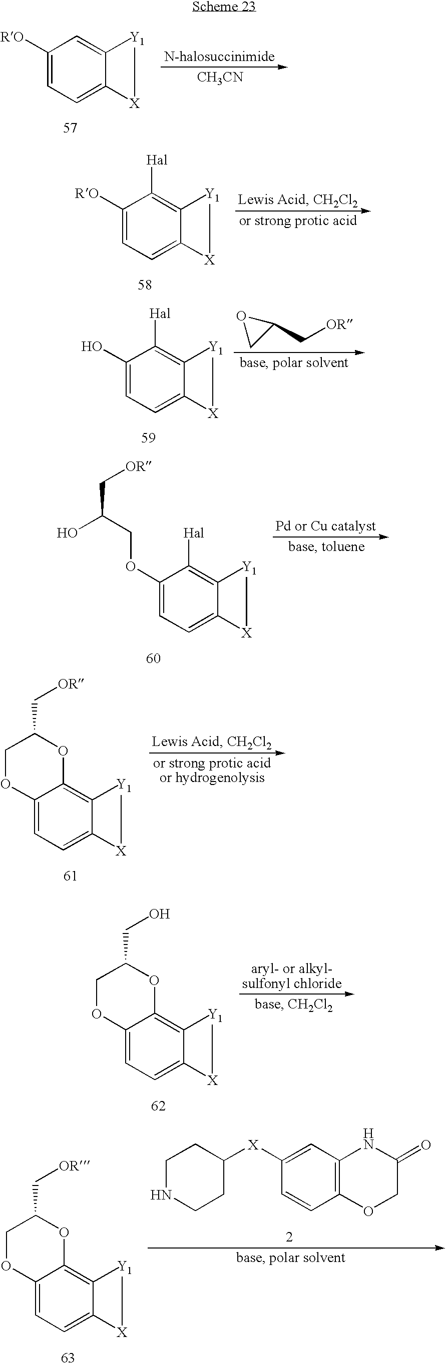

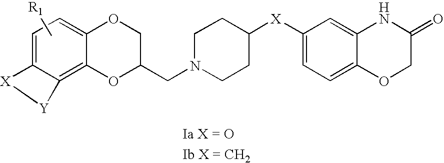

Compounds 2 and 63 can be made by any of the methods described herein or otherwise known to those of skill in the art and used in the final step to produce a compound of formula la or lb. Although Scheme 23 is depicted with formula 2 in the final step, the invention includes methods using other substituted piperidines such as those of formulas 2b, 2c, 2h, 2i, 2j, and 2k, and others. See e.g. Examples 15 and 16 which employ the compounds of formula 2j and 2k, respectively.

Affinity of Compounds of the Invention for the Serotonin Transporter

A protocol similar to that used by Cheetham et. al. (Neuropharmacol. 32:737, 1993) was used to determine the affinity of the compounds of the invention for the serotonin transporter. The compound's ability to displace ³H-paroxetine from male rat frontal cortical membranes was determined using a Tom Tech filtration device to separate bound from free ³H-paroxetine and a Wallac 1205 Beta Plate® counter to quantitate bound radioactivity. Kᵢ values thus determined for standard clinical antidepressants are 1.96 nM for fluoxetine, 14.2 nM for imipramine and 67.6 nM for zimelidine. A strong correlation has been found between ³H-paroxetine binding in rat frontal cortex and ³H-serotonin uptake inhibition.

High affinity for the serotonin 5HT_{1A} receptor was established by testing the claimed compound's ability to displace [³H] 8-OH-DPAT (dipropylamino-tetralin) from the 5HT_{1A} serotonin receptor following a modification of the procedure of Hall et al., J. Neurochem. 44, 1685 (1985) which utilizes CHO cells stably transfected with human 5HT_{1A} receptors. The 5HT_{1A} affinities for the compounds of the invention are reported below as Kᵢ values.

Determination of Antagonist or Antagonist Activity at 5HT_{1A} Receptors

Antagonist or antagonist activity at 5HT_{1A} receptors was established by using a ³⁵S-GTPγS binding assay similar to that used by Lazareno and Birdsall (Br. J. Pharmacol. 109: 1120, 1993), in which the test compound's ability to affect the binding of ³⁵S-GTPγS to membranes containing cloned human 5HT_{1A} receptors was determined. Agonists produce an increase in binding whereas antagonists produce no increase but rather reverse the effects of the standard agonist 8-OH-DPAT. The test compound's maximum inhibitory effect is represented as the Iₘₐₓ, while its potency is defined by the IC₅₀. The results of the three standard experimental test procedures described in the preceding three paragraphs were as follows:

| | 5-HT | 5HT_{1A} | |
|---|---|---|---|
| | Transporter | Receptor | |
| | Affinity | Affinity | 5HT_{1A} Function |
| Compound | Kᵢ (nM) | Kᵢ (nM) | IC₅₀ (nM) (Iₘₐₓ) |
| Example 1 | 9.5 | 0.26 | 59.3 (97) |
| Example 2 | 8.9 | 0.16 | |
| Example 5 | 11.0 | 0.54 | |
| Example 6 | 11.9 | 0.25 | |
| Example 7 | | 0.10 | |
| Example 8 | 18.2 | 1.49 | |
| Example 9 | 4.5 | 3.53 | |
| Example 10 | 58.5 | | |
| Example 11 | 81.0 | | |
| Example 12 | 6.65 | 0.29 | |
| Example 13 | 13.85 | 0.63 | |
| Example 14 | 2.66 | 0.59 | |
| Example 15 | 82.0 | 0.68 | |
| Example 16 | 5.0 | 0.40 | |
| Example 17 | 38.15 | 0.28 | |
| Example 18 | 35.20 | 0.93 | |

Antagonist or Antagonist Activity at 5HT_{1A} Receptors for example compounds 20-26 was determined using the following protocols.

Determination the Affinity of Test Compounds to Inhibit Binding of [3H]-Citalopram to Human Serotonin Transporter (h5HT-T).

### Materials and Methods:

A. Cell Culture (h5HT-T/HEK293)
1. MEDIA REAGENTS: Dulbecco's Modified Eagle Medium (DMEM) with High Glucose, L-Glutamine, 110 mg/L Sodium Pyruvate, Pyridoxine Hydrochloride containing:
a) 10% Fetal Bovine Serum, Heat-inactivated and Dialyzed (10,000 Mol Wt.)
b) 1% Pennicilin-streptomycin
c) 100 μg/ml Geneticin/G418
2. PROCEDURE FOR PASSAGING AND CELL SCALE UP:
a) Cells are grown to 80-90% confluence in T-175 flasks.
b) To split cells, aspirate medium and wash cells with Dulbecco's Phosphate Buffered Saline (D-PBS) without Calcium Chloride and magnesium chloride. Aspirate PBS from cells.
c) Trypsinize cell with 5 ml Trypsin-EDTA (o.o5% Trypsin, 0.53 mM EDTA-4Na) for approximately 1 min.
d) Cells are grown to 80-90% confluence in T-175 flasks.
e) Dislodge cells by tapping the flask or triturating the cells from the plastic. Immediately add 15 ml Cell Culture medium to inactivate trypsin and mix well. Count cells using a hemacytometer if necessary.
f) Add 1 ml of cell suspension to new T-175 culture flasks (to a ˜1:20 dilution of the original flask) containing 30 ml fresh Cell Culture medium and mix gently. (eg. 1×T175 flask can seed 20×T175 flasks)
g) Incubate cells at 37° C., 5% CO₂, in a humidified incubator. NOTE: Cells will grow to confluence within 3-4 days.

### Membrane Preparation:

1. Cells are resuspended in 3 volumes of 50 mM Tris-HC1 (pH 7.4) to 1 volume of initial cell fraction
2. Partially homogenize cells by swirling or by using transfer pipetor to make cell solution more homogenous and aliquot solution into 4 clear 50 ml polypropylene centrifuge tubes
3. Tubes are balanced and spun down at 2,000 rpm for 10 minutes at 4° C. using the HS-4 rotor (rotor code 8)
4. Supernatant is decanted and pellets resuspend in an additional 75 ml of 50 mM Tris-HCl (pH 7.4)→˜18 ml Tris to each of 4 tubes
5. Spin cells again at 5, 000 rpm for minutes at 4° C. using the HS-4 rotor and decant supernatant
6. Resuspend final pellts in appropriate volume of 50 nM Tris to yield protein concentration of ˜6 mg/ml
   add 100 ml Tris to original cell suspension of 35 ml
   add ˜72 ml; Tris to original cell suspension of 25 ml
7. Add the appropriate final volume and split the cells into two/three 50 ml Falcon polypropylene tubes. Thoroughly vortex your cell suspension in these tubes to ensure homogeneity before aliquoting.
8. Pool the samples from the 50 nl tubes into one larger flat-bottom vessel and stir constantly on ice (4° C.).
9. While stirring, aliquot 1 ml portions out into 1.5 ml cryogenic vials.
10. Store vials at −70° C. until subsequtn asay. Retain one aliquot for protein concentration, K_{d} and Bₘₐₓ determination before starting experiments using new batch of cells. Proetin concentration is determined using the Lowry method.

### C. Binding Assay:

1. Required buffers:
Assay buffer; 50 mM Tris HCl, 120 mM NaCl and 5 mM KCl, pH 7.4
2. Binding protocol: Preliminary screening assays are performed using single replicates on duplicate plates. Dose-response binding assays are performed in a single replicates on duplicate OptiPlates (Perkin Elmer) using 0.1 nM to 3 μM of test drug.

a) To each well add 10 μL of test compound, blank compound (10 μM Fluoxetine), or assay buffer (50 mM Tris HCl, 120 mM NaCl and 5 mM KCl, pH 7.4. Each well is then supplemented with 40 μL of assay buffer.

b) To each well add 50 μL of 4 nM of [3H]-Citalopram (to give a final assay concentration of 1 nM). The isotope solution is also disuted in 50 mM Tris HCl, 120 mM NaCl and 5 mM KCl, pH 7.4
c) Resuspend membrane pellet in binding buffer to achieve a solution containing 5 μg of protein in 50 μL. This will yield a final protein concentration of 5 μg/well.
d) To each well add 0.5 mg of Wheatgerm Agglutinin (WGA) SPA beads in 50 μl of 50 mM Tris HCl, 120 mM NaCl and 5 mM KCl, pH 7.4
e) Incubate plates, while gently shaking, for at least 1 hour so as to reach equilibrium. Plates can still be read if left to incubate overnight without loss of signal.
f) Count plates on TopCount.

### Aanalysis of Results

3H-Citallopram binding is expressed as percent of specific binding, with IC50 and Kᵢ VALUES DETERMINED BY nonlinear regression analysis using the Activity Base software package. Specific binding is defined as total binding observed in the presence of 10 μM fluoxetine. Additionally, saturation analysis of the h5HT-T/HEK293 membranes using 3H-Citalopram in the range of 0.1 nM to 10 nM revealed a K_{D} value of 1 nM ±1.3 (SEM) and a Bₘₐₓ of 5.8 pmol/mg±07 (SEM).

Determination of the Inhibitory Activity of Compounds at the Serotonin 5HT1A Receptor Subtype using Scintillation Proximity Assay (SPA) Technology

### Materials and Methods:

Cells: CHO cells expressing the serotonin 5HT1A receptor subtype were grown in suspension in 10 liter bioreactor and supplied as a wet cell pellet

Binding experiments are performed in a total volume of 200 μl using a 96 well microtiter plate format (Packard Optiplate). On the day of the assay, the membranes are thawed and resuspended with enough 50 mM Tris (HCI) pH 7.5, 10 mM MgCl₂, 0.2 mM EDTA, 10 μM pargyline, and 0.1% ascorbate (assay buffer) to achieve a protein concentration of 800 μg/ml. To each well of the microtiter plate, 20 μl of 10× test compound in water containing 3.3% DMSO, 3 nM of [3H]-OH DPAT (Amersham, SA: 200 Ci/mmol), 80 μg cell membrane and assay buffer are combined to achieve a volume of 150 μl. Assay buffer and 10 μM cold 5-HT are substituted for the test compound in separate wells to define ‘total’ and ‘nonspecific’ bindings, respectively. The incubation period is initiated by the addition of 50 μl of 10 mg/ml PVT WGA SPA beads (RPNQ0060, Amersham GE Healthcare) resuspended in assay buffer for a final concentration of 0.5 mg/well. The plates are sealed and gently shaken at room temperature using an orbital shaker (setting 1.5) until equilibrium is achieved (2 h-overnight incubation). Radioactivity (CPM) is measured by Packard TopCount (1 min counting time/well).

### Analysis of Results:

Specific binding is described as the total radioactivity bound less the amount bound in the presence of 10 μM 5HT, referred to as non-specific binding (NSB). Binding in the presence of varying concentrations of test compounds is expressed as percent of specific binding in the absence of compound.

{(Bound−NSB)/(Total−NSB)}×100=% Total

Regression analysis of % bound data from ten concentrations is performed in GraphPad Prism, XL Fit or equivalent software. IC50 values are calculated using a four-parameter logistic curve fitting model and Ki values are calculated by the Cheng-Prusoff equation below:

$$Ki = \frac{IC 50}{1 + L / Kd}$$

where L is the nM concentration of the radioactive ligand used and the Kd is the dissociation constant of the ligand for the receptor. The Kd for [3H-OH DPAT in the SPA binding format is 3 nM.
Single dose

Percent inhibition: The displacement of [3H]5-HT by the test compound at h5-HT1A as determined by: [(Total DPM−compound DPM)/(Total DPM−NSB DPM)]×100.
Conc.: concentration of test compound; NBS: non-specific binding; DPM: disintegrations per min

### REFERENCES

Roberts C., Price G. W., Middlemiss D. N. Ligands for the investigation of 5-HT autoreceptor function, Brain Tesearch Bulletin, 56 (2001) 463-469

Scintillation Proximity Assay Manual, Amersham Biosciences(GE healthcare)

The results of these experimental test procedures described in the preceding paragraphs were as follows:

| | human SERT SPA | h-5HT1A |
|---|---|---|
| | binding assay | binding assay |
| Example | Ki (nm) | Ki (nM) |
| 20 | 5.88 | 1.15 |
| 21a | 68.16 | 15.53 |
| 21b | 1.74 | 1.12 |
| 22a | 60.74 | 14.8 |
| 22b | 2.79 | 0.54 |
| 23 | 12.43 | 2.42 |
| 24 | 9.77 | 2.33 |
| 25 | 146.38 | 13.78 |
| 26 | 375.85 | 360.91 |

### PREPARATION COMPOUNDS OF THE INVENTION

The following examples illustrate the production of representative compounds of this invention. Additional compounds can be made through analogous methods.

### EXAMPLE 1

### 6-[(1-{[(2S)-8-methyl-2,3-dihydro[1,4]dioxino[2,3-f]quinolin-2-yl]methyl}piperidin-4-yl)oxy]-2H-1,4-benzoxazin-3(4H)-one dihydrochloride salt

### Step A. tert-Butyl 4-{[(4-methylphenyl)sulfonyl]oxy}piperidine-1-carboxylate

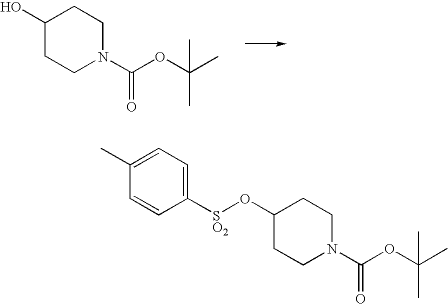

To an ice cold solution of 1-tert-butyloxycarbonyl-4-hydroxypiperidine (3 g, 14.9 mmol) in anhydrous pyridine (8 mL) was added dropwise over 10 minutes a solution of 4-methylphenylsulfonyl chloride (3.36 g, 17.6 mmol) in anhydrous pyridine (6 mL). The yellow solution was allowed to come to room temperature and stirred overnight. An additional amount (0.3 g) of the sulfonyl chloride and a catalytic amount of 4-(dimethylamino)pyridine were added and stirring continued for 5 hours at room temperature. The mixture was cooled in an ice-water bath, quenched with ice-water (60 mL) and extracted with ethyl acetate. The extracts were washed with 1N potassium hydrogen sulfate, water, 5% sodium bicarbonate and water, and dried over anhydrous magnesium sulfate. The colorless solution was evaporated to dryness to provide the title compound as a thick oil that solidified upon standing in vacuo (5.2 g).

HRMS [(+)ESI, m/z]: 356.1543 [M+H]⁺. Calcd for C₁₇H₂₆NO₅S: 356.15262.

### Step B. 4-(3-Oxo-3,4-dihydro-2H-benzo[1,4]oxazin-6-yloxy)-piperidine-1-carboxylic acid tert-butyl ester

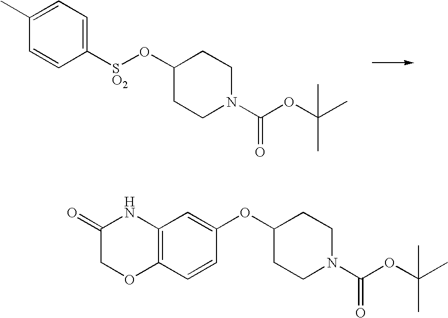

6-Hydroxy-2H-1,4-benzoxazin-3(4H)-one (0.514 g, 3.11 mmol, prepared starting from 6-acetyl-2H-1,4-benzoxazin-3(4H)-one according to the procedure described in WO 2004/089915 A1) was added portionwise to a stirred, ice cold suspension of sodium hydride (60% suspension in oil, 3.11 mmol) kept under nitrogen. After stirring at room temperature for 60 minutes, a solution of tert-butyl 4-{[(4-methylphenyl)sulfonyl]oxy}piperidine-1-carboxylate of step A (1.11 g, 3.11 mmol) in anhydrous N,N-dimethylformamide (5 mL) was added dropwise. The mixture was heated to 55-60° C. for 24 hours. The mixture was poured onto ice (15 mL) and extracted with ethyl acetate. The extracts were washed twice with brine and dried over anhydrous magnesium sulfate. Removal of the solvent provided a light brown oil. The residue was chromatographed on silica Merck-60 using a gradient from 90:10 to 65:35 of hexane/ethyl acetate to provide 0.306 g of unreacted phenol along with the desired title compound (0.337 g, 32%; 76% based on recovered unreacted starting material).

MS [(−)ES, m/z]: 347.1 [M−H]⁻

HPLC (Primesphere CN, 2×15 cm column, detection 254 nm, sample dissolved in ethanol, mobile phase 5% EtOH in hexane,r.t.): Rₜ=4.1 min.

### Step C. 6-(Piperidin-4-yloxy)-4H-benzo[1,4]oxazin-3-one hydrochloride salt

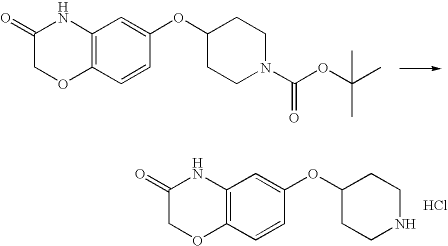

To a solution of 4-(3-oxo-3,4-dihydro-2H-benzo[1,4]oxazin-6-yloxy)-piperidine-1-carboxylic acid tert-butyl ester of Step B (0.238 g, 0.683 mmol) in ethyl acetate (2.5 mL) containing a small amount of dichloromethane was added 1N hydrochloric acid in diethylether (15 mL). The mixture was warmed to 40° C. under nitrogen for 90 minutes. Additional 1N hydrochloric acid in diethylether was added (5 mL) at 90 minutes and at 19 hours. After 20 hours at 40° C. the reaction mixture was cooled, and concentrated to small volume. The residue was treated with diethylether and a small amount of hexane and sonicated to provide an off white solid. The solid was collected, washed with diethylether and dried in vacuo to yield the desired hydrochloride salt of the title compound (0.160 g).

MS [(+)ES, m/z]: 249.1 [M+H]⁺

MS [(−)ES, m/z]: 247.1 [M−H]⁻

### Step D. 6-[(1-{[(2S)-8-Methyl-2,3-dihydro[1,4]dioxino[2,3-f]quinolin-2-yl]methyl}piperidin-4-yl)oxy]-2H-1,4-benzoxazin-3(4H)-one dihydrochloride salt

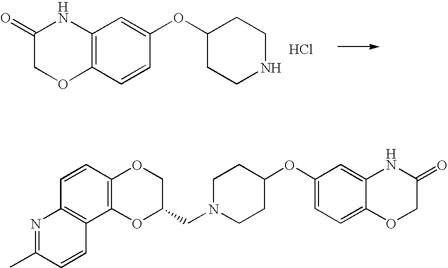

To a stirred solution of the 6-(piperidin-4-yloxy)-4H-benzo[1,4]oxazin-3-one hydrochloride salt of step C (0.140 g, 0.491 mmol) in anhydrous dimethylsulfoxide (3 mL) containing N,N-diisopropylethylamine (1.2 equivalents) kept under nitrogen was added portionwise over 30 minutes [(2R)-8-methyl-2,3-dihydro[1,4]dioxino[2,3-f]quinolin-2-yl]methyl 4-bromobenzenesulfonate (0.243 g, 0.540 mmol). The reaction mixture was stirred at 75-80° C. for ca. 24 hours, cooled, and poured into saturated aqueous sodium bicarbonate. The mixture was extracted with ethyl acetate, and the extracts were washed with brine and dried over anhydrous magnesium sulfate. The residue obtained upon removal of the solvent was chromatographed on silica Merck-60 using hexane-ethyl acetate 1:1 followed by a gradient from 50:49:1 to 50:46:4 of hexane/ethyl acetate/methanolic ammonia to provide the title compound as a colorless glass that solidified upon standing in vacuo (0.079 g). The free base was dissolved in ethyl acetate and converted to the dihydrochloride salt by the addition of 2 equivalents of 1N hydrochloric acid in diethylether. The yellow precipitate was washed with diethylether and hexane and dried in vacuo (0.070 g), m.p. 216-219° C. (dec).

MS (ES) m/z 460.2.

[α]_{D}25=−57.2° (c=1, MeOH)

MS [(+)ES, m/z]: 462.2 [M+H]⁺

MS [(−)ES, m/z]: 460.2 [M−H]⁻

HRMS [(+)ESI,m/z]: 462.2023 [M+H]⁺. Calcd for C₂₆H₂₈N₃O₅: 462.2029.

HPLC (Chromolith Monolith, 0.46×10 cm column, gradient: acetonitrile in water (0.01% TFA): Rt=1.056 min.

### EXAMPLE 2

### 6-[(1-{[(2S)-8-Methyl-2,3-dihydro[1,4]dioxino[2,3-f]quinolin-2-yl]methyl}piperidin-4-yl)methyl]-2H-1,4-benzoxazin-3(4H)-one dihydrochloride

### Step A. (4-Methoxy-3-nitrobenzyl)(triphenyl)phosphonium bromide

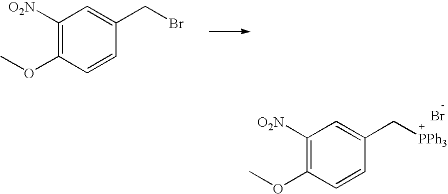

To a solution of 4-(bromomethyl)-1-methoxy-2-nitrobenzene (5.80 g, 23.77 mmol) in toluene (180 mL) was added triphenylphosphine (6.23 g, 23.77 mmol) at room temperature. The reaction mixture was refluxed for 5 hours and cooled to room temperature. The resulting solid was collected by filtrationa nd dried in vacuo to afford the title compound (8.6 g, 71%), mp: 255-257° C.

### Step B. Benzyl 4-(4-methoxy-3-nitrobenzylidene)piperidine-1-carboxylate

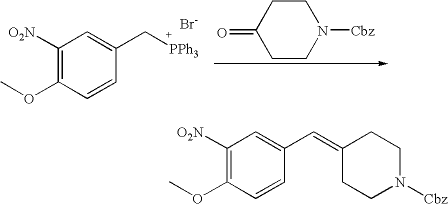

To a solution of (4-methoxy-3-nitrobenzyl)(triphenyl)phosphonium bromide of Step 1(3.75 g, 7.4 mmol) in tetrahydrofuran (35 ml) was added n-butyl lithium (2.96 ml, 2.5 M) at −78° C. To the resulting orange colored suspension was added benzyl 4-oxopiperidine-1-carboxylate. The mixture was stirred at reflux for 2 hours, cooled to room temperature, diluted with water and extracted with ethyl acetate. The organic layer was collected, washed with water and brine, dried over anhydrous magnesium sulfate and concentrated to dryness. The residue was purified by chromatography over silica gel using a gradient form 5 to 15% of ethyl acetate in hexane, to afford the title compound (0.57 g, 21%) as a yellow solid, m.p. 107-108° C.

MS [(+)ES, m/z]: 383 [M+H]⁺

### Step C. Benzyl 4-(4-hydroxy-3-nitrobenzylidene)piperidine-1-carboxylate

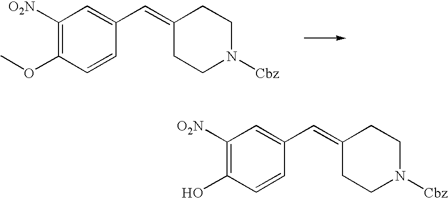

To a solution of benzyl 4-(4-methoxy-3-nitrobenzylidene)piperidine-1-carboxylate of Step B (2 mmol, 0.77 g) in dichloromethane (20 mL) was added boron tribromide at −78° C. The mixture was stirred at room temperature for 40 minutes, treated with methanol (5 mL), stirred for a few minutes and concentrated to dryness. To the residue was added tetrahydrofuran (10 mL), aqueous saturated sodium bicarbonate (10 mL) and benzyl chloroformate (0.682 g, 4 mmol) at 0° C. After stirring at room temperature for 1 hour, piperidine (2.5 ml) was added. The mixture was stirred at room temperature for 30 minutes, acidified to ca. pH 4 and extracted with ethyl acetate. The organic layer was washed with water and brine, dried and concentrated. The residue was purified by chromatography on silica gel (35% ethyl acetate in hexane) to afford the title compound (0.3 g, 41%).

MS [(−)ES, m/z]: 367 [M−H]⁻

### Step D. Benzyl 4-[4-(2-methoxy-2-oxoethoxy)-3-nitrobenzylidene]piperidine-1-carboxylate

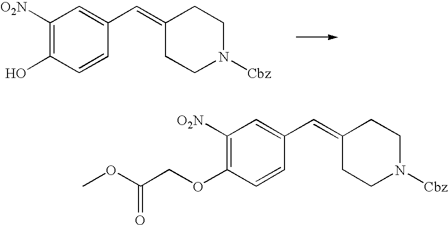

To a solution of benzyl 4-(4-hydroxy-3-nitrobenzylidene)piperidine-1-carboxylate of Step C (1.1 mmol, 0.46 g) in acetonitrile (5 mL) was added methyl bromoacetate (1.1 mmol, 0.1 mL) and potassium carbonate (1.32 mmol, 0.18 g). The mixture was heated at reflux for 15 minutes, cooled to room temperature, treated with water, and extracted with ethyl acetate (2×30 mL). The organic layer was washed with water and brine, dried over anhydrous magnesium sulfate, concentrated and the residue purified by chromatography over silica gel (40% ethyl acetate in hexane) to afford the title compound (0.15 g, 31%).

MS [(+)ES, m/z]: 441 [M+H]⁺.

### Step E. 6-(Piperidin-4-ylmethyl)-2H-1,4-benzoxazin-3(4H)-one

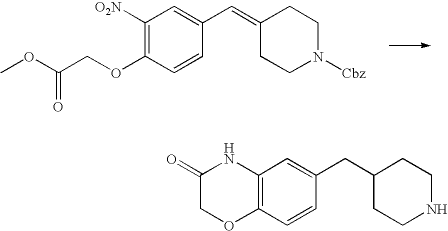

The solution of benzyl 4-[4-(2-methoxy-2-oxoethoxy)-3-nitrobenzylidene]piperidine-1-carboxylate of Step D (0.090 g ) in methanol (15 mL) was heated to 65° C., and Pd/C (0.015 g) added. The mixture was hydrogenated at 50 psi for 18 hours, filtered and concentrated to afford the title compound as an off white solid (0.060 g, 100%), mp: 105-109° C.

MS [(+)ES m/z]: 247 [M+H]⁺

MS [(−)ES m/z]: 245 [M−H]⁻

### Step F. 6-({1-[(8-Methyl-2,3-dihydro[1,4]dioxino[2,3-f]quinolin-2-yl)methyl]piperidin-4-yl}methyl)-2H-1,4-benzoxazin-3(4H)-one dihydrochloride

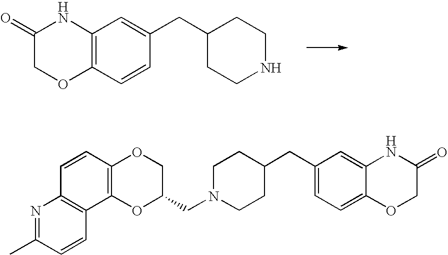

To a solution of [(2R)-8-methyl-2,3-dihydro[1,4]dioxino[2,3-f]quinolin-2-yl]methyl 4-bromobenzenesulfonate (0.119 g, 0.26 mmol) in dimethylsulfoxide (1 mL) was added 6-(piperidin-4-ylmethyl)-2H-1,4-benzoxazin-3(4H)-one of Step E (65 mg, 0.26 mmol) and N,N-diisopropylethyl amine (0.040 g, 0.312 mmol). The reaction mixture was heated at 80° C. overnight, cooled to room temperature, diluted with dichloromethane, and basified with aqueous soidium bicarbonate. The organic layer was washed with water and brine, dried over anhydrous magnesium sulfate and concentrated to a residue that was purified by chromatography over silica gel (5% methanolic ammonia in dichloromethane} to afford the desired compound. The dihydrochloride salt was prepared as a yellow solid, mp: 215° C. (dec.), by adding 1N hydrochloric acid in ether to a methanolic solution of the free base

MS [(+)ES, m/z): 460 [M+H]⁺

MS [(−)ES, m/z): 458 [M−H]⁻

HRMS: [(+)ESI, m/z]: 460.2234. Calcd for C₂₇H₃₀N₃O₄: 460.2236 [M+H]⁺.

### EXAMPLE 3

### 6-[(1-{[(2S)-8-methyl-2,3-dihydro[1,4]dioxino[2,3-f]quinolin-2-yl]methyl}piperidin-4-yl)oxy]-2H-1,4-benzoxazin-3(4H)-one dihydrochloride salt

### Step A. 6-Hydroxy-2H-1,4-benzoxazin-3(4H)-one

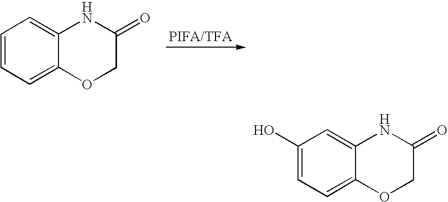

To 2H-1,4-benzoxazin-3(4H)-one (4.5 g, 0.03 mole) in trifluoroacetic acid (90 mL), under nitrogen at room temperature, was added at all once a solution of [bis(trifluoroacetoxy)iodo]benzene (PIFA) (1.2 eq, 0.036 mole, 15.5 g) in trifluoroacetic acid (80 mL). The reaction mixture was quickly brought to reflux and stirred under reflux for 20 min. The reaction mixture, while still very warm, was poured over ice-H₂O (600 mL) and the precipitate that formed was filtered, dissolved in EtOAc-MeOH and concentrated to generate slightly impure product. The filtrated was extracted with EtOAc (2×). The organic extracts were pooled, treated with brine, dried over anhydrous MgSO₄, filtered and concentrated to generate crude desired product 1. Precipitation from EtOAc-EtOH generated a batch of pure desired product. The remainder of the crude material was purified by flash column chromatography using (19:1) CH₂Cl₂-MeOH followed by (14:1) CH₂Cl₂-MeOH as elution solvent. A total of 3.5 g (70% yield) of desired product was isolated as off-white to pale brown solid.

LC-MS: [M+H]⁺m/z=166.1 and [M−H]⁻ m/z=164.1; ¹HNMR/DMSO-d₆: consistent for desired product.

### Step B. tert-Butyl 4-{[(2-nitrophenyl)sulfonyl]oxy}piperidine-1-carboxylate

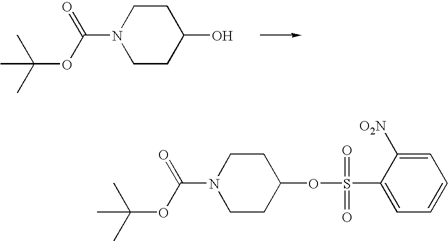

To an ice-cold solution of tert-butyl-4-hydroxy-1-piperidinecarboxylate (10.0 g, 0.049 mole), 2-nitrobenzenesulfonyl chloride (1.2 eq, 12.9 g, 0.059 mole) and 4-(dimethylamino)pyridine (0.05 eq, 0.3 g, 0.0024 mole) in anhydrous methylene chloride (60 mL) was added dropwise over 20 minutes triethylamine (2.5 eq, 17.4 mL, 0.124 mole). The solution was warmed to room temperature and stirred overnight. The precipitate was filtered off, washed with CH₂Cl₂ and the filtrate concentrated. The residue was extracted with ether, washed with saturated sodium bicarbonate (3×), dried over anhydrous MgSO₄, filtered and concentrated to generate the crude product as a light brown oil. The crude product was chromatographed on silica Merck-60 using a gradient from (9:1) to (3:1) of Hex/EtOAc to provide 12.75 g (66% yield) of pure desired product as a thick yellow oil which solidified upon standing.

¹H NMR/DMSO-d₆: consistent for desired product.

### Step C. tert-Butyl 4-[(3-oxo-3,4-dihydro-2H-1,4-benzoxazin-6-yl)oxy]piperidine-1-carboxylate

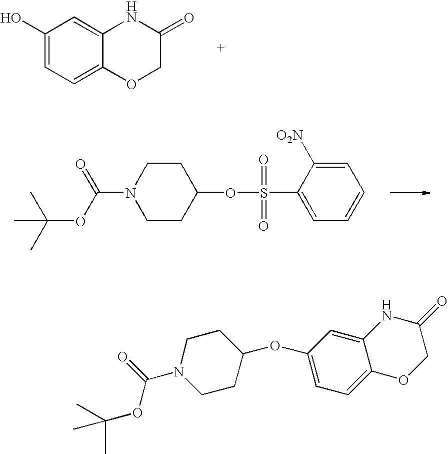

To 6-hydroxy-2H-1,4-benzoxazin-3(4H)-one of Step A (2.5 g, 0.015 mole) in anhydrous DMF (20 mL), under nitrogen at room temperature, was added cesium carbonate (1.8 eq, 0.027 mole, 8.8 g). The reaction mixture was stirred at room temperature for 30 min and at 40-45° C. for another 30 min. Then, tert-butyl 4-{[(2-nitrophenyl)sulfonyl]oxy}piperidine-1-carboxylate of Step B (1.8 eq, 0.027 mole, 10.6 g) dissolved in anhydrous DMF (30 mL) was added dropwise over a 45 min period (about ¾ of the solution). The reaction mixture was stirred at 40° C. for 30 min and the remainder of the nosylate solution added dropwise over a 15 min period. The reaction mixture was stirred at 40° C. for another 3 hrs at which time more cesium carbonate (0.2 eq, 0.003 mole, 0.97 g) was added. The reaction mixture was then stirred at 40° C. overnight. It was cooled to room temperature and poured over ice-H₂O (500 mL). It was made neutral with saturated NH₄Cl and extracted with EtOAc (2×). The organic extracts were pooled, treated with brine, dried over anhydrous MgSO₄, filtered and concentrated to generate the crude product. The crude product was purified on Isco Combi-Flash Retrieve system using a 120 g silica gel column and (1:1) Hex-EtOAc as elution solvent. 2.48 g (48% yield) of desired product was isolated as a pale yellow solid. The product was found to be identical to the product of Example 1, Step B.

LC-MS: [M−H]⁻ m/z=347.1

LC (10 to 100% CH₃CN): >96% pure

¹HNMR/DMSO-d₆: consistent for desired product

### Step D. 6-(Piperidin-4-yloxy)-4H-benzo[1,4]oxazin-3-one hydrochloride salt

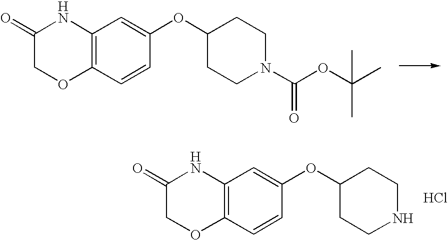

The mixture of 4-(3-oxo-3,4-dihydro-2H-benzo[1,4]oxazin-6-yloxy)-piperidine-1-carboxylic acid tert-butyl ester of Step C (2.45 g, 0.007 mole) and 1.25 N hydrochloric acid in ethanol (25 equiv, 0.175 mole, 140 mL) was refluxed under nitrogen for 45 minutes. The reaction mixture was cooled, and concentrated to dryness. The yellow residue was triturated with diethylether and a small amount of hexane and sonicated to provide an off white solid. The solid was collected, washed with diethylether and hexane, and dried in vacuo to yield the desired hydrochloride salt of the title compound, m.p. 186° C.

MS [(−)ES, m/z]: 247.1 [M−H]⁻

Anal. (C₁₃H₁₆N₂O₃.1 HCl. 0.25 H₂O) C,H,N.

### Step E. 6-[(1-{[(2S)-8-Methyl-2,3-dihydro[1,4]dioxino[2,3-f]quinolin-2-yl]methyl}piperidin-4-yl)oxy]-2H-1,4-benzoxazin-3(4H)-one dihydrochloride salt

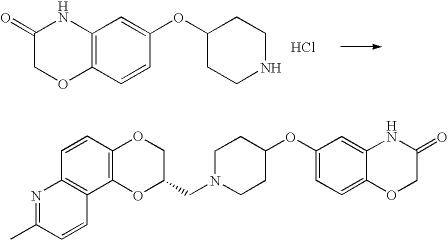

The 6-(piperidin-4-yloxy)-4H-benzo[1,4]oxazin-3-one hydrochloride salt of step D was converted to the title compound according to the procedure of Example 1, Step D.

[α]_{D}²⁵=−49.6° (c=1% solution, DMSO)

¹H NMR (DMSO-d₆): consistent for desired product

MS (APPI) m/z 462

Anal. Calcd for C₂₆H₂₇N₃O₅. 1.90 HCl. H₂O: C, 56.90; H, 5.68; N, 7.66. Found: C, 57.36; H, 5.58; N, 7.81.

### EXAMPLE 4

### 6-[(1-{[(2S)-8-Methyl-2,3-dihydro[1,4]dioxino[2,3-f]quinolin-2-yl]methyl}piperidin-4-yl)oxy]-2H-1,4-benzoxazin-3(4H)-one dihydrochloride salt

### Step A. 1-{[(2S)-8-Methyl-2,3-dihydro[1,4]dioxino[2,3-f]quinolin-2yl]methyl}piperidin-4-ol

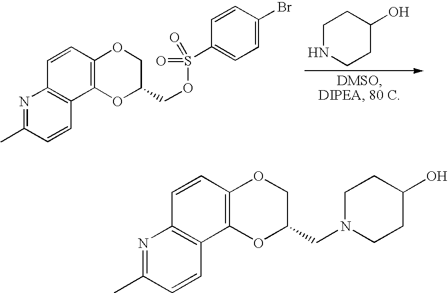

A mixture of [(2R)-8-methyl-2,3-dihydro[1,4]dioxino[2,3-f]quinolin-2-yl]methyl 4-bromobenzenesulfonate (45 g, 100 mmol) and 4-hydroxypiperidine (25.3 g, 250 mmol) in anhydrous DMSO (150 ml) was stirred under nitrogen at 87° C. for 2 hours, monitoring by HPLC (the peak of the bromobenzene sulfonate disappeared after ca. 1.5 h). The resulting red-brown clear solution was cooled down to room temperature and poured at stirring into 1.3 L of aqueous NaHCO₃ (1L of conc. and 300 ml of water). The resulting somewhat cloudy warm solution (the mixing is slightly exothermic) was stirred at room temperature for 3 h, upon which time a crystalline precipitate came out. The solid was filtered, washed with water, suspended in acetonitrile, filtered and air-dried overnight to give 27.4 g (87%) of the the title compound as beige crystals.

¹HNMR was found to be consistent for the desired product.

### Step B. 6-[(1-{[(2S)-8-methyl-2,3-dihydro[1,4]dioxino[2,3-f]quinolin-2-yl]methyl}piperidin-4-yl)oxy]-2H-1,4-benzoxazin-3(4H)-one

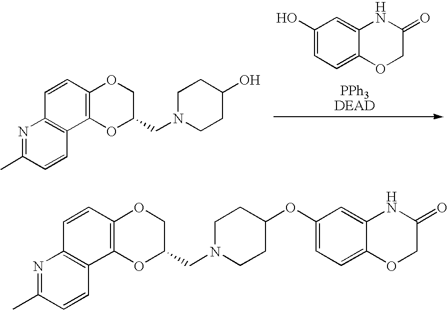

To a mixture of 1-{[(2S)-8-methyl-2,3-dihydro[1,4]dioxino[2,3-f]quinolin-2yl]methyl}piperidin-4-ol of Step A (12.5 g, 40 mmol), 6-hydroxy-2H-1,4-benzoxazin-3(4H)-one of Example 3, Step A (4.4. g, 27 mmol), and triphenylphosphine (10.48 g, 40 mmol) in 150 ml of anhydrous THF under nitrogen at room temperature was added dropwise a solution of diethyl azodicarboxylate (7.0 g, 40 mmol) in 30 ml of anhydrous THF. The exothermic reaction was controlled by cooling as needed. At the end of the addition the initial suspension turned into a dark-brown clear solution. To that solution were added 50 ml of 2N HCl in isopropanol. The yellow precipitate was filtered, washed with isopropanol, dissolved in 100 ml of 30% aq. acetic acid, the solution extracted with dichloromethane (50 ml), basified with 10N NaOH to pH 5-6, and extracted at that pH with dichloromethane (3×100 ml). The combined organic extracts were washed with aqueous NaHCO₃ (2×50 ml), dried over sodium sulfate, evaporated, and purified on silica gel, loading in methylene chloride, washing with methylene chloride/ethyl acetate 1:1 (1 L), ethyl acetate (1 L), and ethyl acetate/methanol/triethylamine 95:5:1 (1 L). The fractions containing product were combined, evaporated, crystallized from ethyl acetate/MTBE 4:1, and the white crystals washed with MTBE/heptane 1:1 to yield the title compound (6.5 g, 52%), m. p. 149-150° C. This material corresponds to the free base of the hydrochloride salt described in Examples 1 and 3.

### EXAMPLE 5

### 4-Methyl-6-[(1-{[(2S)-8-methyl-2,3-dihydro[1,4]dioxino[2,3-f]quinolin-2-yl]methyl}piperidin-4-yl)oxy]-2H-1,4-benzoxazin-3(4H)-one dihydrochloride salt

### Step A. tert-Butyl 4-[(4-methyl-3-oxo-3,4-dihydro-2H-1,4-benzoxazin-6-yl)oxy]piperidine-1-carboxylate

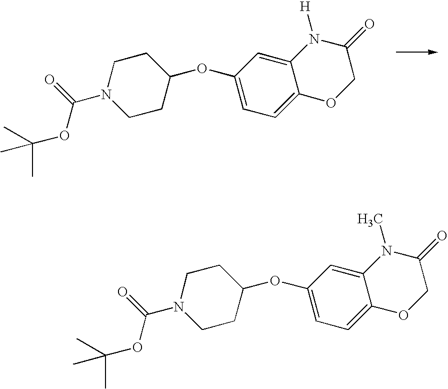

To tert-butyl 4-[(3-oxo-3,4-dihydro-2H-1,4-benzoxazin-6-yl)oxy]piperidine-1-carboxylate of Example 3, Step C (0.33 g, 0.94 mmol) in anhydrous DMF (6.5 mL), under nitrogen at room temperature, was added cesium carbonate (1.1 eq, 1.03 mmole, 0.33 g). The reaction mixture was stirred at room temperature for 15 minutes and at 40° C. for 15 minutes. Iodomethane (1.2 eq, 1.13 mmol, 70 μL) was added and the reaction mixture stirred at 40° C. for 2 hrs. More iodomethane (0.2 eq, 0.11 mmol, 7 μL) was added and the reaction mixture stirred at 40° C. for another 30 min. The reaction mixture was quenched with H₂O and made neutral with saturated NH₄Cl. It was extracted with EtOAc (2×). The organic extracts were pooled, back-washed once with H₂O, washed with brine, dried over anhydrous MgSO₄, filtered and concentrated to generate the crude product. The crude product was purified on Isco Combi-Flash Retrieve system using a 40 g silica gel column and (3:2) Hex-EtOAc as elution solvent. 0.267 g (78% yield) of desired product was isolated as a gum.

MS: [M+H]⁺m/z=363.1

¹HNMR/DMSO-d₆ was found to be consistent for the desired product.

### Step B. 4-Methyl-6-(piperidin-4-yloxy)-2H-1,4-benzoxazin-3(4H)-one hydrochloride salt

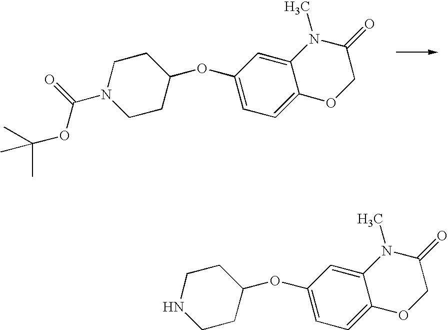

To tert-butyl 4-[(4-methyl-3-oxo-3,4-dihydro-2H-1,4-benzoxazin-6-yl)oxy]piperidine-1-carboxylate of Step A (0.32 g, 0.88 mmol) was added 1.25N HCl/EtOH (10 eq, 8.8 mmol, 7 mL). The reaction mixture was brought to reflux and kept under reflux for 1.5 hrs. It was cooled to room temperature and concentrated. The residue was triturated with Et₂O/hexane and the precipitate was filtered and washed thoroughly with Et₂O/hexane. It was then dried under vacuum generating the hydrochloride salt of the title compound, m.p. 215° C. (dec)

MS (ES) m/z 263.1

¹HNMR (DMSO-d₆) was found to be consistent for the desired product.

Anal. Calcd for C₁₄H₁₈N₂O₃.1.10 HCl: C, 55.60; H, 6.37; N, 9.26. Found: C, 55.32; H, 6.04; N, 9.28.

### Step C. 4-Methyl-6-[(1-{[(2S)-8-methyl-2,3-dihydro[1,4]dioxino[2,3-f]quinolin-2-yl]methyl}piperidin-4-yl)oxy]-2H-1,4-benzoxazin-3(4H)-one dihydrochloride salt

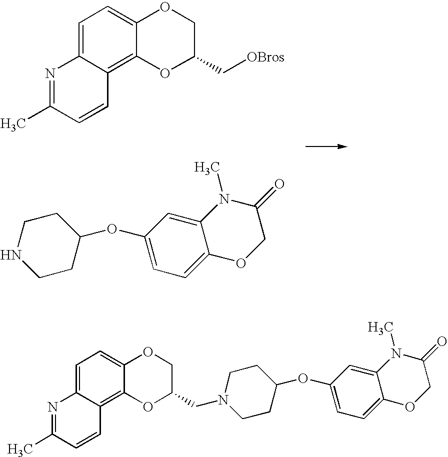

To 4-methyl-6-(piperidin-4-yloxy)-2H-1,4-benzoxazin-3(4H)-one hydrochloride salt of Step B (0.19 g, 0.63 mmol) in n-butanol (5 mL), under nitrogen at room temperature, was added N,N-diisopropylethylamine (3 eq, 1.89 mmol, 0.33 mL). The reaction mixture was brought to 118-120° C. and [(2R)-8-methyl-2,3-dihydro[1,4]dioxino[2,3-f]quinolin-2-yl]methyl 4-bromobenzenesulfonate (1.2 eq, 0.75 mmol, 0.34 g) added portionwise over a 15 minute period. The reaction mixture was then stirred at 118-120° C. overnight. After cooling to room temperature, it was diluted with EtOAc and washed once with H₂O. The aqueous layer was extracted once more with EtOAc. The organic extracts were pooled, treated with brine, dried over anhydrous MgSO₄, filtered and concentrated to generate the crude product. The crude product was purified on Isco Combi-Flash Retrieve system using a 40 g silica gel column and (6:3:1) Hex-EtOAc-MeOH (1% NH₄OH) as elution solvent. The title compound (0.170, g 57% yield) was isolated as a yellowfoam. It was then converted to the dihydrochloride salt by dissolution in THF and addition of 1M HCl/Et₂O (2.4 eq) generating a yellow brown solid, mp 164° C. (dec). [α]_{D}²⁵=−34.2° (c=1% solution, DMSO).

¹H NMR (DMSO-d6) was found to be consistent for the desired product

MS (ES) m/z 476.1

Anal. Calcd for C₂₇H₂₉N₃O₅.2.00 HCl.1.20 H₂O: C, 56.89; H, 5.91; N, 7.37. Found: C, 56.86; H, 6.07; N, 7.21.

### EXAMPLE 6

### 6-[4-({[(2S)-8-Methyl-2,3-dihydro[1,4]dioxino[2,3-f]quinolin-2-yl]methyl}amino)butoxy]-2H-1,4-benzoxazin-3(4H)-one dihydrochloride salt

### Step A. 6-(4-Bromobutoxy)-2H-1,4-benzoxazin-3(4H)-one

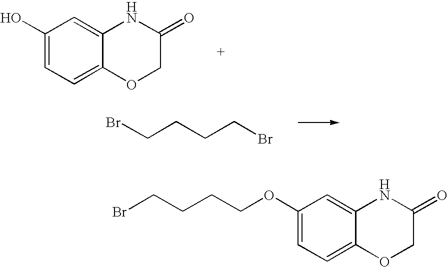

To 6-hydroxy-2H-1,4-benzoxazin-3(4H)-one (0.25 g, 1.51 mmol) in anhydrous acetonitrile (8 mL), under nitrogen at room temperature, was added potassium carbonate (1 eq, 1.51 mmol, 208 mg) and 1,4-dibromobutane (1.1 eq, 1.66 mmol, 0.2 mL). The slurry was brought to reflux (80-82 ° C.) and stirred at that temperature overnight. The reaction mixture was cooled to room temperature and quenched with saturated NH₄Cl. It was extracted with EtOAc (2×), the organic extracts pooled, treated with brine, dried over anhydrous MgSO₄, filtered and concentrated to generate the crude product. The crude product was purified on Isco Combi-Flash Retrieve system using a 40 g silica gel column and (3:1) Hex-EtOAc as elution solvent. The title compound (0.179 g,40% yield) was isolated as a white solid.

¹HNMR/DMSO-d₆: consistent for desired product.

### Step B. 6-[4-({[(2S)-8-Methyl-2,3-dihydro[1,4]dioxino[2,3-f]quinolin-2-yl]methyl}amino)butoxy]-2H-1,4-benzoxazin-3(4H)-one dihydrochloride salt

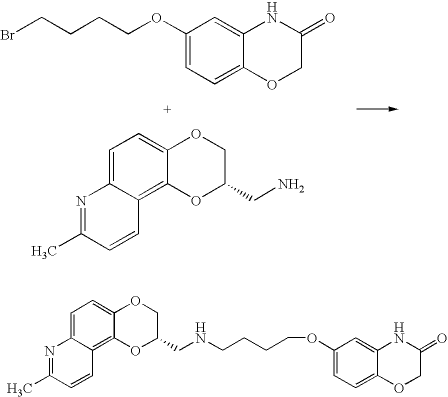

To 1-[(2S)-8-methyl-2,3-dihydro[1,4]dioxino[2,3-f]quinolin-2-yl]methanamine (1.4 eq, 0.348 mmol, 80 mg) in n-butanol (3 mL), under nitrogen at room temperature, was added N,N-diisopropylethylamine (3 eq, 0.747 mmol, 0.13 mL) and a solution of 6-(4-bromobutoxy)-2H-1,4-benzoxazin-3(4H)-one of Step B (1 eq, 0.249 mmol, 75 mg) in n-BuOH (3 mL). The reaction mixture was brought to 118-120° C. and kept at that temperature over the weekend. The reaction mixture was then cooled down to room temperature and quenched with saturated NH₄Cl. It was extracted with EtOAc (2×), the organic extracts pooled, treated with brine, dried over anhydrous MgSO₄, filtered and concentrated to generate the crude product. The crude product was purified on Isco Combi-Flash Retrieve system using a 40 g silica gel column and (5:4:1) EtOAc-Hex-MeOH (1% NH₄OH) as elution solvent. The pure title compound (0.058 g, 51% yield) was isolated as a yellow gum along with 0.024 g of bis-alkylated product. The title compound was converted to the dihydrochloride salt by dissolution in EtOAc and addition of 1M HCl/Et₂O (2.4 eq) generating a yellow-brown solid, m.p. 143 ° C. (dec). [α]_{D}²⁵=−46.4° (c=1% solution, DMSO)

¹H NMR (DMSO-d₆) was found to be consistent for the desired product

MS [(−)ES, m/z]: 448.2 [M−H]⁻

Anal. Calcd for C₂₅H₂₇N₃O₅.2.00 HCl . H₂O: C, 55.56; H, 5.78; N, 7.78. Found: C, 55.55; H, 5.66; N, 7.62.

### EXAMPLE 7

### 6-[3-({[(2S)-8-Methyl-2,3-dihydro[1,4]dioxino[2,3-f]quinolin-2-yl]methyl}amino)propoxy]-2H-1,4-benzoxazin-3(4H)-one dihydrochloride salt

### Step A. 6-(3-Bromopropoxy)-2H-1,4-benzoxazin-3(4H)-one

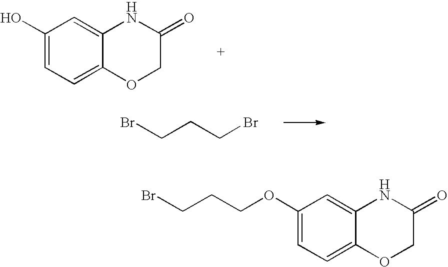

A mixture of 6-hydroxy-2H-1,4-benzoxazin-3(4H)-one (545 mg, 3.3 mmol) and potassium carbonate (1 eq, 3.36 mmol, 465 mg) in anhydrous acetonitrile (34.5 mL) was left to stir at room temperature under nitrogen for 15 minutes. 1,3-Dibromopropane (1.2 eq, 3.96 mmol, 0.4 mL) was then added dropwise over 15 minutes. The resulting mixture was then heated to 82° C. and stirred at that temperature overnight. The reaction mixture was cooled to room temperature and poured into ice water. It was then extracted with EtOAc (4×), the organic extracts pooled, treated with brine, dried over anhydrous MgSO₄, filtered and concentrated to generate the crude product. The crude product was chromatographed on silica Merck-60 using (1:1) Hex-EtOAc as elution solvent to yield the pure title compound (0.183 g, 16% yield).

¹H NMR/DMSO-d₆ was found to be consistent for the desired product

MS [(−)ES m/z]: 284 [M−H]⁻

### Step B. 6-[3-({[(2S)-8-Methyl-2,3-dihydro[1,4]dioxino[2,3-f]quinolin-2-yl]methyl}amino)propoxy]-2H-1,4-benzoxazin-3(4H)-one dihydrochloride salt

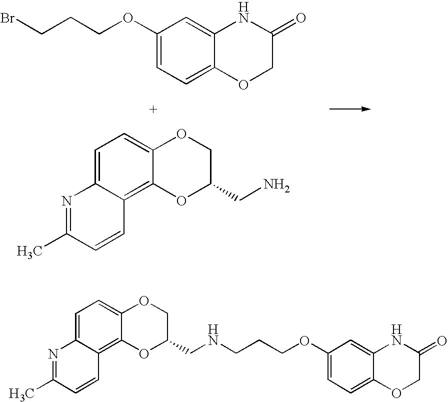

To 6-(3-bromopropoxy)-2H-1,4-benzoxazin-3(4H)-one of Step A (182.9 mg, 0.639 mmol) in anhydrous dimethylsulfoxide (2.21 mL), under nitrogen at room temperature, was added 1-[(2S)-8-methyl-2,3-dihydro[1,4]dioxino[2,3-f]quinolin-2-yl]methanamine (191.3 mg, 0.831 mmol) in anhydrous dimethylsulfoxide (2.21 mL) and triethylamine (0.72 mL). The reaction mixture was then heated to 90° C. and kept at that temperature overnight. The reaction mixture was cooled to room temperature and diluted with EtOAc (40 mL). It was washed with water (3×), treated with brine, dried over anhydrous MgSO₄, filtered and concentrated to generate the crude product. The residue was chromatographed on silica Merck-60 using 95:5 CH₂Cl₂/MeOH to provide pure title compound as a fluffy beige solid (0.070 g, 25% yield). The latter was converted to the dihydrochloride salt by dissolution in EtOAc and addition of 1M HCl/Et₂O (2 eq) generating a yellow solid, m.p. 184° C. (dec).

¹H NMR (DMSO-d₆) was found to be consistent for the desired product.

MS [+)(ES, m/z]:=436.1 [M+H]⁺

Anal. Calcd for C₂₄H₂₅N₃O₅.2.00 HCl.1.6 H₂O: C, 53.66; H, 5.67; N, 7.82. Found: C, 53.26; H, 5.52; N, 7.61.

### EXAMPLE 8

### 6-{[(3S)-1-{[(2S)-8-Methyl-2,3-dihydro[1,4]dioxino[2,3-f]quinolin-2-yl]methyl}pyrrolidin-3-yl]oxy}-2H-1,4-benzoxazin-3(4H)-one dihydrochloride salt

### Step A. tert-Butyl (3R)-3-{[(2-nitrophenyl)sulfonyl]oxy}pyrrolidine-1-carboxylate

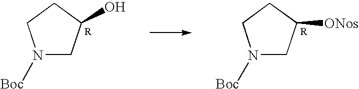

To an ice-cold solution of tert-butyl (3R)-3-hydroxypyrrolidine-1-carboxylate (1.0 g, 5.34 mmol), 2-nitrobenzenesulfonyl chloride (1.42 g, 6.41 mmol) and 4-(dimethylamino)pyridine (0.032 g) in anhydrous methylene chloride (6.44 mL) was added dropwise over 20 minutes triethylamine (1.86 mL, 13.35 mmol). The solution was then left to warm to room temperature and was stirred overnight. The precipitate was filtered, washed with CH₂Cl₂ and the filtrate concentrated. The residue was taken up in EtOAc/H₂O and extracted with EtOAc. The organic extracts were washed with saturated NaHCO₃ (3×), dried over anhydrous MgSO₄, filtered and concentrated to generate the crude product. The residue was chromatographed on silica Merck-60 using (1:1) hexane/EtOAc to provide pure title compound as a thick yellow oil (1.46 g, 73% yield). ¹H NMR/DMSO-d₆ was found to be consistent for the desired product.

### Step B. tert-Butyl (3S)-3-[(3-oxo-3,4-dihydro-2H-1,4-benzoxazin-6-yl)oxy]pyrrolidine-1-carboxylate

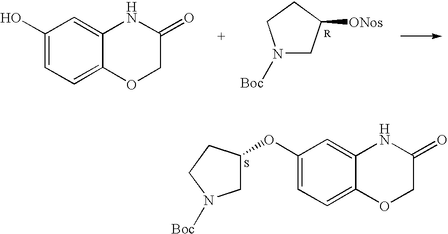

6-hydroxy-2H-1,4-benzoxazin-3(4H)-one (88.7 mg, 0.537 mmol) and cesium carbonate (350 mg, 1.074 mmol) were stirred in anhydrous DMF (2.31 mL) under nitrogen at room temperature for 30 minutes. tert-Butyl (3R)-3-{[(2-nitrophenyl)sulfonyl]oxy}pyrrolidine-1-carboxylate of Step A (0.2 g, 0.537 mmol) in anhydrous DMF (2.32 mL) was then added dropwise over 10 minutes. The reaction mixture was brought to 40° C. and stirred at that temperature overnight. The mixture was then cooled to room temperature and poured into ice water. It was extracted with EtOAc (4×) and the organic extracts were washed with H₂O, treated with brine, dried over anhydrous MgSO₄, filtered and concentrated to generate the crude product. The residue was chromatographed on silica Merck-60 using (1:1) hex/EtOAc as elution solvent to provide the title compound (0.079 g, 44% yield) as a colorless oil.

¹H NMR/DMSO-d₆ was found to be consistent for the desired product

MS [(−)ES, m/z]: 333 [M−H]⁻

### Step C. Synthesis of 6-[(3S)-pyrrolidin-3-yloxy]-2H-1,4-benzoxazin-3(4H)-one

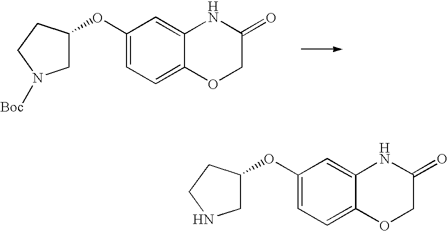

To tert-butyl (3S)-3-[(3-oxo-3,4-dihydro-2H-1,4-benzoxazin-6-yl)oxy]pyrrolidine-1-carboxylate of Step A (462 mg, 1.38 mmol) was added 1.25 N HCl/EtOH (5.52 mL). The mixture was brought to reflux and kept under reflux for 45 minutes. It was cooled down to room temperature and concentrated to remove the ethanol. The off-white residue was triturated with Et₂O and a little hexane. The fluffy off-white precipitate was then filtered and washed with Et₂O/hexane. It was dried under vacuum generating the hydrochloride salt of the title compound (330 mg, 92% yield).

¹H NMR/DMSO-d₆ was found to be consistent for the desired product

MS [(+)ES, m/z]: 235 [M+H]⁺; MS [(−)ES, m/z]: 233 [M−H]⁻

To 6-[(3S)-pyrrolidin-3-yloxy]-2H-1,4-benzoxazin-3(4H)-one hydrochloride salt (343 mg, 1.267 mmol) in anhydrous methanol (2.58 mL) was added two scoops of Amberlyst 21 ion exchange resin. The mixture was then left to stir overnight at room temperature. The resin was filtered off and washed with methanol followed by CH₂Cl₂/iPrOH (4:1). The filtrate was concentrated to give the title compound (free base) as an off-white solid (159.9 mg, 53% yield).

¹H NMR/DMSO-d₆ was found to be consistent for the desired product

MS [(+)ES, m/z]: 235 [M+H]⁺; MS [(−)ES, m/z]: 233 [M−H]⁻

### Step D. 6-{[(3S)-1-{[(2S)-8-Methyl-2,3-dihydro[1,4]dioxino[2,3-f]quinolin-2-yl]methyl}pyrrolidin-3-yl]oxy}-2H-1,4-benzoxazin-3(4H)-one dihydrochloride salt

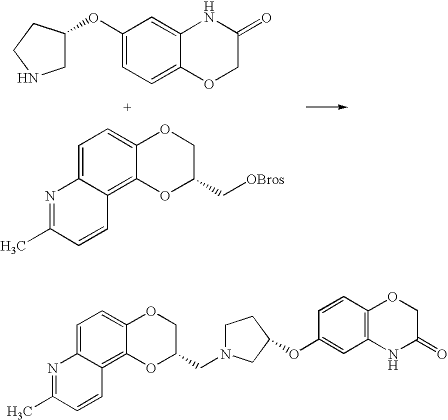

To 6-[(3S)-pyrrolidin-3-yloxy]-2H-1,4-benzoxazin-3(4H)-one of Step C (free base) (159.9 mg, 0.683 mmol) in n-butanol (4.01 mL) under nitrogen at room temperature was added N,N-diisopropylethylamine (0.36 mL). The mixture was heated to 120° C. and [(2R)-8-methyl-2,3-dihydro[1,4]dioxino[2,3-f]quinolin-2-yl]methyl 4-bromobenzenesulfonate (307.6 mg, 0.683 mmol) added portionwise over 35 minutes. The reaction mixture was kept at 120° C. for 48 hours. It was cooled to room temperature, poured over saturated NaHCO₃ solution and extracted with EtOAc (3×). The organic extracts were pooled, treated with brine, dried over anhydrous MgSO₄, filtered and concentrated to generate the crude product. The residue was chromatographed on silica Merck-60 using (3:1) EtOAc/heane followed by (6:3:1) hexane-EtOAc-MeOH (1% NH₄OH) to provide the pure dtitle compound as a white solid (200 mg, 68% yield). Approximately 80 mg of the free base was converted to the dihydrochloride salt by dissolution in EtOAc and addition of 1M HCl/Et₂O (2 eq) generating a yellow solid, m.p. 183° C. (dec).

[α]_{D}²⁵=−37.8° (c=1% solution, DMSO)

¹H NMR (DMSO-d₆) was found to be consistent for the desired product

MS [(−)ES, m/z]: 446.2 [M−H]⁻

Anal. Calcd for C₂₅H₂₅N₃O₅.2.00HCl.1.4 H₂O: C, 55.03; H, 5.50; N, 7.70. Found: C, 55.24; H, 5.50; N, 7.37.

### EXAMPLE 9

### 6-{[(3R)-1-{[(2S)-8-Methyl-2,3-dihydro[1,4]dioxino[2,3-f]quinolin-2-yl]methyl}pyrrolidin-3-yl]oxy}-2H-1,4-benzoxazin-3(4H)-one dihydrochloride salt

### Step A. tert-Butyl (3S)-3-{[(2-nitrophenyl)sulfonyl]oxy}pyrrolidine-1-carboxylate

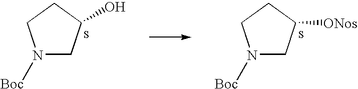

To tert-butyl (3S)-3-hydroxypyrrolidine-1-carboxylate (500 mg, 2.67 mmol), 2-nitrobenzenesulfonyl chloride (709 mg, 3.20 mmol) and 4-(dimethylamino)pyridine (16 mg) in anhydrous dichloromethane (3.22 mL) was added dropwise over 20 minutes triethylamine (0.93 ml, 6.68 mmol). The solution was then left to warm to room temperature and stirred overnight. The precipitate was filtered, washed with CH₂Cl₂ and the filtrate concentrated. The residue was taken up in EtOAc/H₂O and extracted with EtOAc (2×). The organic extracts were washed with saturated NaHCO₃ (3×), dried over anhydrous MgSO₄, filtered and concentrated to generate the crude product. The residue was chromatographed on silica Merck-60 using (1:1) hex-EtOAc to provide the title compound as a thick yellow oil (662.8 mg, 67% yield).

¹H NMR/DMSO-d₆ was found to be consistent for the desired product.

### Step B. tert-Butyl (3R)-3-[(3-oxo-3,4-dihydro-2H-1,4-benzoxazin-6-yl)oxy]pyrrolidine-1-carboxylate

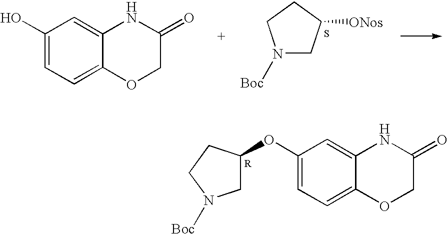

6-Hydroxy-2H-1,4-benzoxazin-3(4H)-one (539.7 mg, 3.268 mmol) and cesium carbonate (2.13 g, 6.536 mmol) were stirred in anhydrous DMF (14.1 mL) under nitrogen at room temperature for 30 minutes. tert-Butyl (3S)-3-{[(2-nitrophenyl)sulfonyl]oxy}pyrrolidine-1-carboxylate of Step A (1.24 g, 3.286 mmol) in anhydrous DMF (14.1 mL) was added dropwise over 10 minutes. The reaction mixture was brought to 40° C. and stirred at that temperature overnight. The mixture was then cooled to room temperature and poured into ice water. It was then extracted with EtOAc (4×) and the organic extracts were pooled, washed with H₂O, treated with brine, dried over anhydrous MgSO₄, filtered and concentrated to generate the crude product. The residue was chromatographed on silica Merck-60 using (1:1) hex/EtOAc as elution solvent to provide the pure title compound (267 mg, 25% yield) as a colorless oil.

¹H NMR/DMSO-d₆ was found to be consistent for the desired product

MS [(−)ES, m/z]: 333 [M−H]⁻

### Step C. 6-[(3R)-Pyrrolidin-3-yloxy]-2H-1,4-benzoxazin-3(4H)-one hydrochloride salt

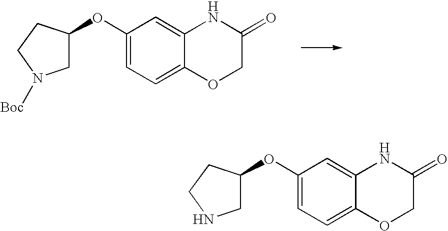

To tert-butyl (3R)-3-[(3-oxo-3,4-dihydro-2H-1,4-benzoxazin-6-yl)oxy]pyrrolidine-1-carboxylate of Step B (267.2 mg, 0.799 mmol) was added 1.25 N HCl/EtOH (3.20 mL). The mixture was brought to reflux and kept under reflux for 45 minutes. It was cooled to room temperature and concentrated to remove ethanol. The residue was then triturated with ether and hexane. The resulting off-white precipitate was filtered and washed with ether/hexane. It was then dried under vacuum generating the hydrochloride salt of the title compound (172.6 mg, 80% yield). mp 180° C. (dec).

[α]_{D}²⁵=+18.00° (c=1% solution, DMSO)

MS [(+)ES, m/z]: 235.1 [M+H]⁺

¹H NMR/DMSO-d₆ was found to be consistent for the desired product.

Anal. Calcd for C₁₂H₁₄N₂O₃. HCl: 0.30 H₂O: C, 52.20; H, 5.69; N, 10.15. Found: C, 52.52; H, 5.22; N, 10.07.

### Step D. 6-{[(3R)-1-{[(2S)-8-Methyl-2,3-dihydro[1,4]dioxino[2,3-f]quinolin-2-yl]methyl}pyrrolidin-3-yl]oxy}-2H-1,4-benzoxazin-3(4H)-one dihydrochloride salt

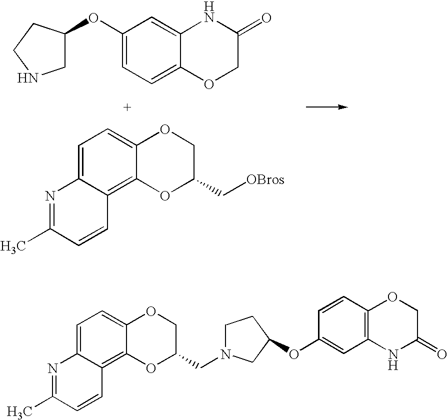

To 6-[(3R)-pyrrolidin-3-yloxy]-2H-1,4-benzoxazin-3(4H)-one hydrochloride salt of Step C (172.6 mg, 0.638 mmol) in n-butanol (3.75 mL) under nitrogen at room temperature was added N,N-diisopropylethylamine (0.33 mL). The mixture was heated to reflux (˜118° C.) and [(2R)-8-methyl-2,3-dihydro[1,4]dioxino[2,3-f]quinolin-2-yl]methyl 4-bromobenzenesulfonate (287.3 mg, 0.638 mmol) was added portionwise over 35 minutes. The mixture was kept at 120° C. for 48 hours, then cooled to room temperature, poured over saturated NaHCO₃ solution and extracted with EtOAc (3×). The organic extracts were pooled, treated with brine, dried over anhydrous MgSO₄, filtered and concentrated to generate the crude desired product. The residue was chromatographed on silica Merck-60 using (3:1) EtOAc-hex followed by (6:3:1) hex-EtOAc-MeOH (1% NH₄OH) to provide the pure title compound as a beige solid (143 mg, 52% yield). Approximately 75 mg of the free base was converted to the dihydrochloride salt by dissolution in EtOAc and addition of 1M HCl/Et₂O (2 eq) generating a yellow solid, m.p. 183° C. (dec)

[α]_{D}²⁵=−77.4° (c=1% solution, DMSO)

¹H NMR (DMSO-d₆) was found to be consistent for the desired product

MS[(−)ES, m/z]: 446.2 [M−H]⁻

Anal. Calcd for C₂₅H₂₅N₃O₅.2.00 HCl.1.8 H₂O: C, 54.32; H, 5.58; N, 7.60. Found: C, 53.87; H, 5.42; N, 7.34.

### EXAMPLE 10

### 6-[4-(Methylamino)butoxy]-2H-1,4-benzoxazin-3(4H)-one hydrochloride salt

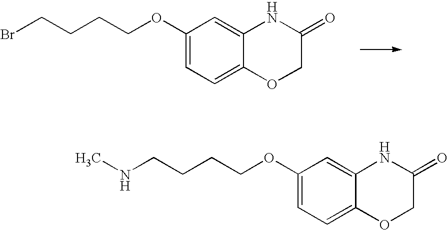

To 6-(4-bromobutoxy)-2H-1,4-benzoxazin-3(4H)-one of Example 7, Step A (125 mg, 0.416 mmol) in absolute EtOH (2 mL) was added a 40% aq solution of methylamine (0.8 mL). The reaction was stirred at room temperature for 48 hrs. It was concentrated and the residue taken up in EtOAc/1N NaOH. The organic layer was separated and the aqueous layer extracted once more with EtOAc. The organic extracts were pooled, treated with brine, dried over anhydrous MgSO₄, filtered and concentrated to generate the crude product. The crude product was purified by flash column chromatography using (9:1) CH₂Cl₂-MeOH (1% NH₄OH) followed by (4:1) CH₂Cl₂-MeOH (1% NH₄OH) as elution solvent. The title compound (72 mg, 70% yield) was isolated as a gummy solid that was then converted to the hydrochloride salt by dissolution in EtOH and addition of 1.25N HCl/EtOH (1.2 eq) generating a white solid, m.p. 210-212° C.

MS [(+)ES, m/z]: 251.1 [M+H]⁺

¹HNMR/DMSO-d₆ was found to be consistent for the desired product.

Anal. Calcd for C₁₃H₁₈N₂O₃. HCl: C, 54.45; H, 6.68; N, 9.77. Found: C, 54.16; H, 6.67; N, 9.70.

### EXAMPLE 11

### 6-[3-(Methylamino)propoxy]-2H-1,4-benzoxazin-3(4H)-one

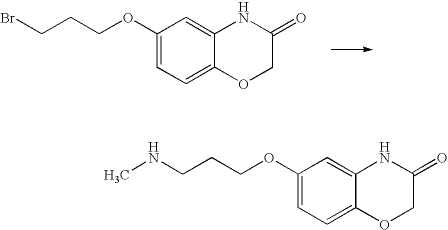

To 6-(3-bromopropoxy)-2H-1,4-benzoxazin-3(4H)-one of Example 7, Step A (127 mg, 0.447 mmol) in absolute EtOH (1.4 mL) was added a 40% aq solution of methylamine (0.72 mL). The reaction was stirred at room temperature for 4 days. It was worked up as described for Example 10. The crude product was purified by flash column chromatography using (9:1) CH₂Cl₂-MeOH (1% NH₄OH) followed by (4:1) CH₂Cl₂-MeOH (1% NH₄OH) as elution solvent. The title compound (40 mg, 38% yield) was isolated as a gum and was then converted to the hydrochloride salt by dissolution in EtOH and addition of 1.25N HCl/EtOH (1.2 eq) generating a white solid, m.p. 193-194° C.

MS [(+)ES, m/z]: 237.1 [M+H]⁺

¹HNMR/DMSO-d₆ was found to be consistent for the desired product.

Anal. Calcd for C₁₂H₁₆N₂O₃. HCl.0.60 H₂O: C, 50.83; H, 6.47; N, 9.88. Found: C, 50.85; H, 6.22; N, 9.91.

### EXAMPLE 12

### 4-Methyl-6-[(1-{[(2S)-8-methyl-2,3-dihydro[1,4]dioxino[2,3-f]quinolin-2-yl]methyl}piperidin-4-yl)methyl]-2H-1,4-benzoxazin-3(4H)-one dihydrochloride salt

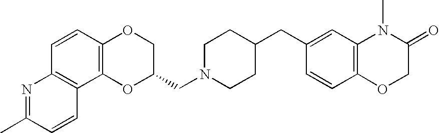

To a solution 6-[(1-{[(2S)-8-methyl-2,3-dihydro[1,4]dioxino[2,3-f]quinolin-2-yl]methyl}piperidin-4-yl)methyl]-2H-1,4-benzoxazin-3(4H)-one of Example 2 (143 mg, 0.31 mmol, free base) in DMF (2 mL) was added NaH (60% dispersion, 14.8 mg, 0.37 mmol). After stirring for 30 minutes, Mel (0.31 mmol, 0.019 ml) was added. The mixture was stirred overnight, then water was added and the aqueous mixture was extracted with EtOAc. The organic layer was washed with water and brine, dried over MgSO₄, filtered, and concentrated. Purification by SiO₂ gel chromatography [CH₂Cl₂/MeOH (saturated with NH₃) 95:5] afforded 0.11 g (78%) of the title compound as the free base. The dhydrochloride salt was prepared by the addition of HCl (1N in Et₂O) to as solution of the free base in MeOH as a yellow solid, m.p. 205-208° C.

MS [(+)ES, m/z]: 474 [M+H]⁺

### EXAMPLE 13

### 4-Methyl-6-[2-(1-{[(2S)-8-methyl-2,3-dihydro[1,4]dioxino[2,3-f]quinolin-2-yl]methyl}piperidin-4-yl)ethyl]-2H-1,4-benzoxazin-3(4H)-one dihydrochloride salt

### Step A. tert-Butyl 4-[2-(4-methyl-2-oxo-3,4-dihydro-2H-1,4-benzoxazin-6-yl)ethyl]piperidine-1-carboxylate

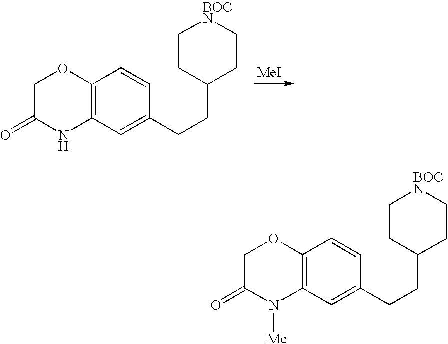

Using essentially the same procedure described for Step A of Example 15, the title compound was obtained from tert-butyl 4-[2-(2-oxo-3,4-dihydro-2H-1,4-benzoxazin-6-yl)ethyl]piperidine-1-carboxylate of Example 14, Step F in 99% yield as a white foam.

MS [(+), m/z]: 397.2 [M+Na]⁺

### Step B. 4-Methyl-6-(2-piperidin-4-ylethyl)-3,4-dihydro-2H-1,4-benzoxazin-2-one hydrochloride salt

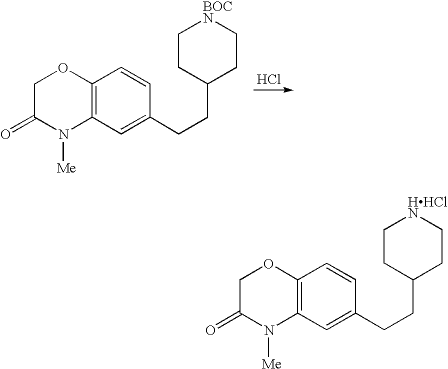

Using essentially the same procedure described for Step G of Example 14, the title compound was obtained in 88% yield from tert-butyl 4-[2-(4-methyl-2-oxo-3,4-dihydro-2H-1,4-benzoxazin-6-yl)ethyl]piperidine-1-carboxylate of Step A as a white foam, m.p. 232-233° C.

MS [(+), m/z}: 275 [M+H]⁺

### Step C. 4-Methyl-6-[2-(1-{[(2S)-8-methyl-2,3-dihydro[1,4]dioxino[2,3-f]quinolin-2-yl]methyl}piperidin-4-yl)ethyl]-2H-1,4-benzoxazin-3(4H)-one dihydrochloride salt

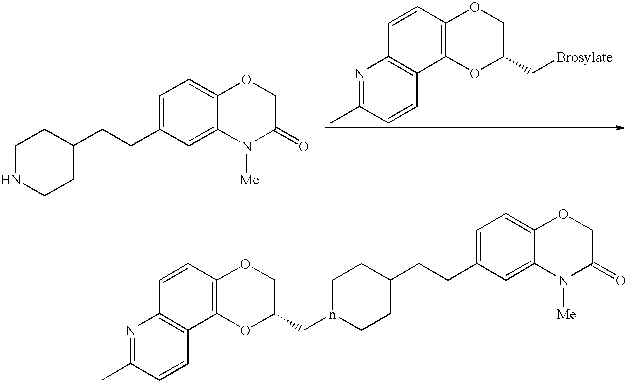

Using essentially the same procedure described for Step H of Example 14, the title compound was obtained from 4-methyl-6-(2-piperidin-4-ylethyl)-3,4-dihydro-2H-1,4-benzoxazin-2-one of Step B (free base) as a yellow solid in 65% yield, m.p. 185-189° C.

MS [(+), m/z]: 488 [M+H]⁺

### EXAMPLE 14

### 6-[2-(1-{[(2S)-8-Methyl-2,3-dihydro[1,4]dioxino[2,3-f]quinolin-2-yl]methyl}piperidin-4-yl)ethyl]-2H-1,4-benzoxazin-3(4H)-one dihydrochloride salt

### Step A. Diethyl (4-methoxy-3-nitrobenzyl)phosphonate

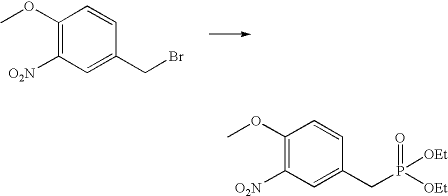

To 4-(bromomethyl)-1-methoxy-2-nitrobenzene (10 g, 40.9 mmol ) was added triethyl phosphate (10.5 g, 63 mmol). The reaction was heated at 120° C. for 3 h and purified by chromatography to afford the desired compound as colorless oil (9.0 g, 72%)

MS [(+), m/z]: 304 [M+H]⁺.

### Step B. Benzyl 4-[2-(4-methoxy-3-nitrophenyl)vinyl]piperidine-1-carboxylate

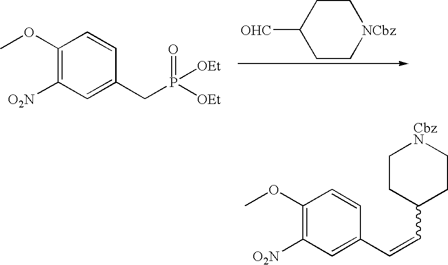

Using essentially the same procedure described for Step A of Example 16, the title compound was obtained from diethyl (4-methoxy-3-nitrobenzyl)phosphonate of Step A and 4-formyl-N-benzyloxycarbonyl-piperidine as colorless oil in 47% yield.

MS [(+), m/z]: 397 [M+H]⁺

### Step C. tert-Butyl 4-[2-(4-hydroxy-3-nitrophenyl)vinyl]piperidine-1-carboxylate

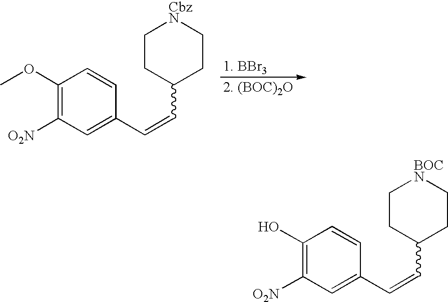

Using essentially the same procedure described for Step B of Example 16, the title compound was obtained from benzyl 4-[2-(4-methoxy-3-nitrophenyl)vinyl]piperidine-1-carboxylate of Step B in 69% yield as a yellow foam.

MS [(−),m/z]: 347 [M−H]⁻

### Step D. tert-Butyl 4-{2-[4-(2-methoxy-2-oxoethoxy)-3-nitrophenyl]vinyl}piperidine-1-carboxylate

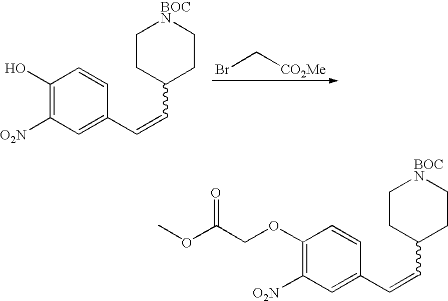

Using essentially the same procedure described for Step C of Example 16, the title compound was obtained from tert-butyl 4-[2-(4-hydroxy-3-nitrophenyl)vinyl]piperidine-1-carboxylate of Step C in 92% yield as a yellow foam.

MS [(+), m/z]: 443 [M+Na]⁺

### Step E. tert-Butyl 4-[2-(2-oxo-3,4-dihydro-2H-1,4-benzoxazin-6-yl)vinyl]piperidine-1-carboxylate

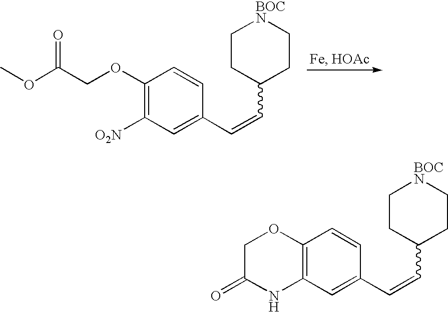

Using essentially the same procedure described for Step D Example 16, the title compound was obtained from tert-butyl 4-{2-[4-(2-methoxy-2-oxoethoxy)-3-nitrophenyl]vinyl}piperidine-1-carboxylate of Step D in 76% yield as a white solid, m.p. 182-183° C.

MS [(−), m/z]: 359 [M−H]⁻

### Step F. tert-Butyl 4-[2-(2-oxo-3,4-dihydro-2H-1,4-benzoxazin-6-yl)ethyl]piperidine-1-carboxylate

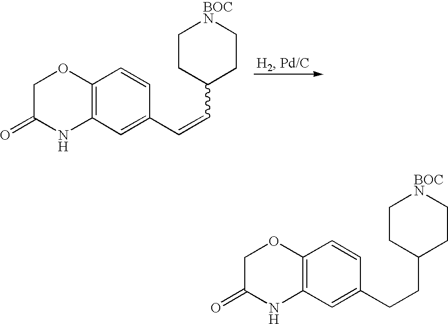

Using essentially the same procedure described for Step E of Example 16, the title compound was obtained from tert-butyl 4-[2-(2-oxo-3,4-dihydro-2H-1,4-benzoxazin-6-yl)vinyl]piperidine-1-carboxylate of Step D in 89% yield as a white foam.

MS [(−), m/z]: 359 [M−H]⁻

### Step G. 6-(2-piperidin-4-ylethyl)-3,4-dihydro-2H-1,4-benzoxazin-2-one hydrochloride salt

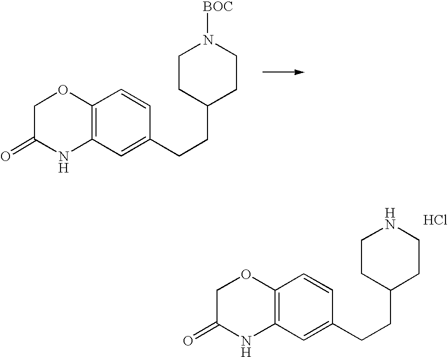

To a solution of tert-butyl 4-[2-(2-oxo-3,4-dihydro-2H-1,4-benzoxazin-6-yl)ethyl]piperidine-1-carboxylate of Step F (0.65 g, 1.8 mmol) in EtOH (5 ml) was added HCl (1N in Et₂O, 9 ml). After stirring at 40° C. for 3 hours, the mixture was cooled to room temperature and set in the refrigerator overnight. The title compound 0.46 g (87%) was collected by vacuum filtration.

MS [(+), m/z]: 261 [M+H]⁺

### Step H. 6-[2-(1-{[(2S)-8-Methyl-2,3-dihydro[1,4]dioxino[2,3-f]quinolin-2-yl]methyl}piperidin-4-yl)ethyl]-2H-1,4-benzoxazin-3(4H)-one dihydrochloride salt

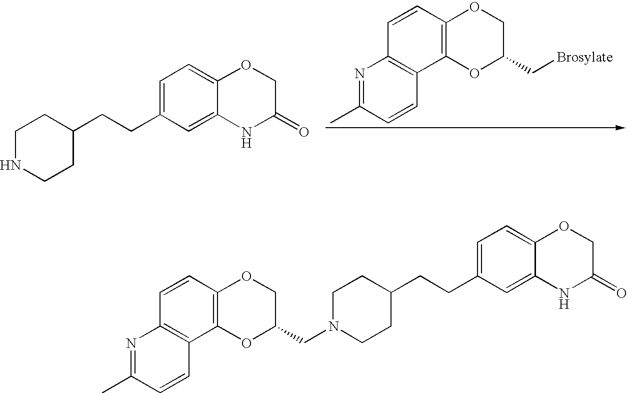

To the suspension of 6-(2-piperidin-4-ylethyl)-3,4-dihydro-2H-1,4-benzoxazin-2-one hydrochloride salt of Step G (0.296 g, 1 mmol) in n-butanol (8 ml) was added [(2R)-8-methyl-2,3-dihydro[1,4]dioxino[2,3-f]quinolin-2-yl]methyl 4-bromo benzene sulfonate (0.54 g, 1.2 mmol), and N-ethyl-N-isopropylpropan-2-amine (0.46 g, 3.6 mmol). The resulting mixture was heated at reflux for 18 hours, cooled to room temperature and the n-butanol removed in vacuo. To the resulting residue was added water and aqueous NaHCO₃, and the mixture was extracted with EtOAc. The organic layer was washed with water and brine, dried over MgSO₄, filtered and concentrated. Purification by SiO₂ gel chromatography [CH₂Cl₂/MeOH (saturated with NH₃) 95:5) afforded of the title compound as the free base (0.28 g, 59%). The dihydrochloride salt was prepared the addition of HCl (1 N in Et₂O) to a methanolic solution of the free base as a yellow solid, m.p. 202-205° C.

MS [(−), m/z]: 472 [M−H]⁻

### EXAMPLE 15

### 4-Methyl-6-[(1-{[(2S)-8-methyl-2,3-dihydro[1,4]dioxino[2,3-f]quinolin-2-yl]methyl}piperidin-3-yl)methyl]-2H-1,4-benzoxazin-3(4H)-one dihydrochloride salt

### Step A. tert-Butyl 3-[(4-methyl-2-oxo-3,4-dihydro-2H-1,4-benzoxazin-6-yl)methyl]piperidine-1-carboxylate

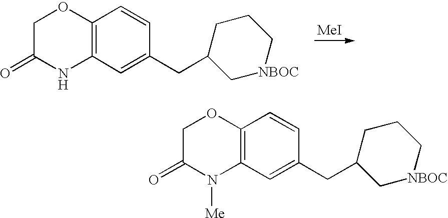

To a solution of tert-butyl 3-[(2-oxo-3,4-dihydro-2H-1,4-benzoxazin-6-yl)methyl]piperidine-1-carboxylate of Step E, Example 16 (0.17 g, 0.49 mmol ) in N,N-dimethylformamide (2.5 ml) was added NaH (60% dispersion in oil, 39 mg, 0.98 mmol). The mixture was stirred at room temperature for 0.5 hours and Mel (0.061 ml, 0.98 mmol) was added. After stirring at room temperature for 5 hours, water was added and the mixture was extracted with EtOAc. The organic layer was washed with water and brine, dried over MgSO₄ and concentrated. Purification by SiO₂ gel chromatography afforded 0.16 g (91%) of the title compound as a colorless oil.

MS [(+), m/z]: 361 [M+H]⁺

### Step B. 4-Methyl-6-(piperidin-3-ylmethyl)-3,4-di hydro-2H-1,4-benzoxazin-2-one hydrochloride salt

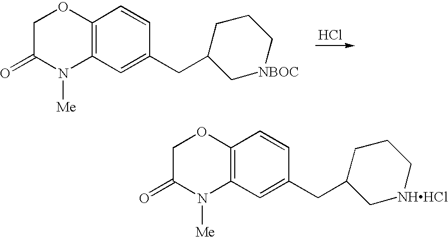

Using essentially the same procedure described for Step G of Example 14, the desired compound was obtained from tert-butyl 3-[(4-methyl-2-oxo-3,4-dihydro-2H-1,4-benzoxazin-6-yl)methyl]piperidine-1-carboxylate of Step B as a white foam

MS [(+), m/z]: 261 [M+H]⁺

### Step C. 4-Methyl-6-[(1-{[(2S)-8-methyl-2,3-dihydro[1,4]dioxino[2,3-f]quinolin-2-yl]methyl}piperidin-3-yl)methyl]-2H-1,4-benzoxazin-3(4H)-one dihydrochloride salt

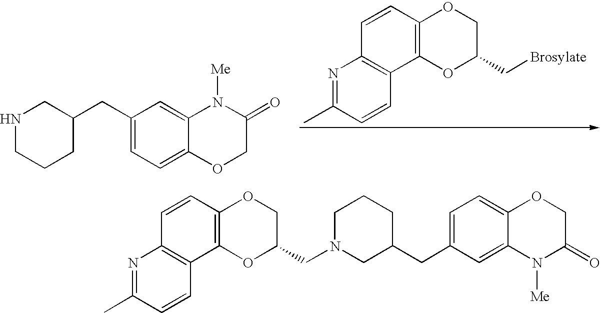

Using essentially the same procedure described for Step H of Example 14, the title compound was obtained from 4-methyl-6-(piperidin-3-ylmethyl)-3,4-dihydro-2H-1,4-benzoxazin-2-one of Step B (free base) in 43% yield as a yellow solid, m.p. 249-251° C.

MS [(+), m/z]: 474 [M+H]⁺

### EXAMPLE 16

### 6-[(1-{[(2S)-8-Methyl-2,3-dihydro[1,4]dioxino[2,3-f]quinolin-2-yl]methyl}piperidin-4-yl)methyl]-2H-1,4-benzoxazin-3(4H)-one dihydrochloride salt

### Step A. tert-Butyl 3-(4-methoxy-3-nitrobenzylidene)piperidine-1-carboxylate

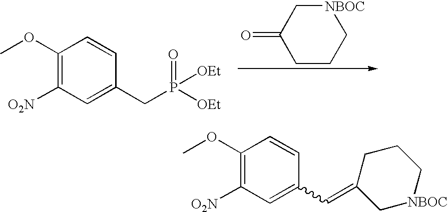

To a −5° C. solution of diethyl (4-methoxy-3-nitrobenzyl)phosphonate (6.06 g, 20 mmol) and 15-crown-15 (0.4 ml, 2 mmol) in 50 ml THF was added NaH (60% dispersion, 1 g, 24 mmol). After stirring at room temperature for 30 minutes, a solution of tert-butyl 3-oxopiperidine-1-carboxylate (4.0, 20 mmol) in THF (10 ml) was added. The reaction was stirred overnight, then was diluted with water and extracted with EtOAc. The organic layer was washed with water and brine, dried over MgSO₄, filtered, and concentrated. Purification by SiO₂ chromatography afforded 2.4 g (49%) of the title compound as a white foam.

MS [(+), m/z]: 349 [M+H]⁺

### Step B. tert-Butyl 3-(4-hydroxy-3-nitrobenzylidene)piperidine-1-carboxylate

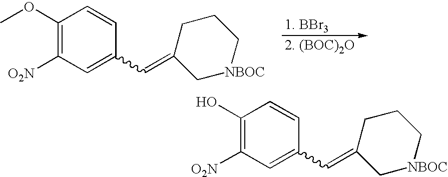

To a −78° C. solution of tert-butyl 3-(4-methoxy-3-nitrobenzylidene)piperidine-1-carboxylate of Step A (2.4 g, 6.89 mmol) in dichloromethane (50 ml) was added boron tribromide (1.3 ml, 13.78 mmol ) dropwise. The reaction was warmed to room temperature and stirred for 1 hour. After re-cooling to −78° C., methanol (15 ml) was added dropwise. The reaction was again allowed to warm to room temperature and stirred for 1 hour, then it was concentrated. The residue was dissolved in 20% H₂O/THF and (BOC)₂O (1.5 g, 6.89 mmol) and triethylamine (1.725 ml, 12.4 mmol) were added. After stirring at RT for 2 hours, the aqueous mixture was extracted with EtOAc. The organic layer was washed with water and brine, then was dried over MgSO₄, filtered and concentrated. Purification by SiO₂ gel chromatography afforded 0.52 g (22%) of the title compound as a yellow oil.

MS [(−), m/z]: 333 [M−H]⁻

### Step C. tert-Butyl 3-[4-(2-methoxy-2-oxoethoxy)-3-nitrobenzylidene]piperidine-1-carboxylate

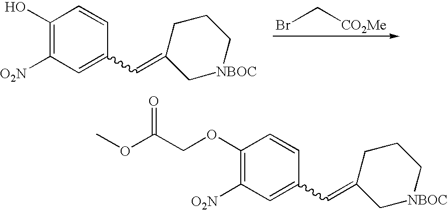

To tert-butyl 3-(4-hydroxy-3-nitrobenzylidene)piperidine-1-carboxylate of Step B (0.52 g, 1.56 mmol) in a mixture of acetone (10 ml) and DMF (1.2 ml) was added methyl bromoacetate (0.44 ml, 4.67 mmol), Cs₂CO₃ (1.54 g, 4.67 mmol), and Nal (0.11 g, 0.78 mmol). After stirring at room temperature for 1 hour, water was added and the acetone was removed under vacuum. The resulting aqueous mixture was extracted with EtOAc. The organic layer was washed with water and brine, dried over MgSO₄, filtered and concentrated. Purification by SiO₂ gel chromatography afforded 0.5 g (79%) of the title compound as a yellow oil.

MS [(+), m/z]: 429 [M+Na]⁺

### Step D. tert-Butyl 3-[(2-oxo-3,4-dihydro-2H-1,4-benzoxazin-6-yl)methylene piperidine-1-carboxylate

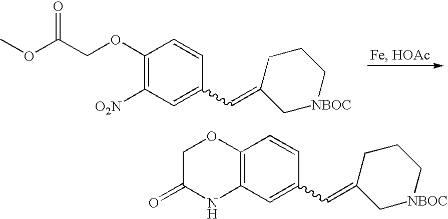

To a solution of tert-butyl 3-[4-(2-methoxy-2-oxoethoxy)-3-nitrobenzylidene]piperidine-1-carboxylate of Step C (0.5 g, 1.23 mmol) in a mixture of HOAc (7 ml) and EtOH (7 ml) was added iron (0.2 g, 3.69 mmol). After heating at 90° C. for 2 hours, the mixture was cooled to room temperature and filtered through the celite. The filtrate was concentrated to a residue which was purified by chromatography to afford 0.32 g (76%) of the title compound as a white foam.

MS [(+), m/z}: 343 [M+H]⁺

### Step E. tert-Butyl 3-[(2-oxo-3,4-dihydro-2H-1,4-benzoxazin-6-yl)methyl]piperidine-1-carboxylate

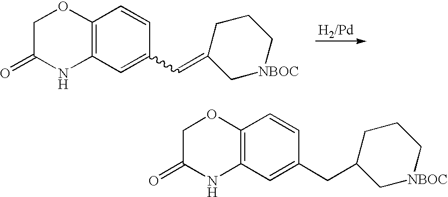

To a solution of tert-butyl 3-[(2-oxo-3,4-dihydro-2H-1,4-benzoxazin-6-yl)methylene]piperidine-1-carboxylate of Step D (0.32 g, 0.93 mmol) in EtOH (15 mL) was added 10% Pd/C (40 mg). After hydrogenation at 40 psi for 4.5 h the catalyst was removed by filtration and the filtrate was concentrated to afford 0.27 g (84%) of the title compound as a white foam.

MS [(−), m/z]: 345 [M−H]⁻

### Step F. 6-(Piperidin-3-ylmethyl)-2H-1,4-benzoxazin-3(4H)-one hydrochloride salt

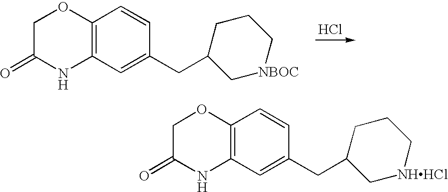

Using essentially the same procedure described for Step G of Example 14, the title compound was obtained from tert-butyl 3-[(2-oxo-3,4-dihydro-2H-1,4-benzoxazin-6-yl)methyl]piperidine-1-carboxylate of Step E in quantitative yield as a white foam.

MS [(−), m/z]: 245 [M−H]⁻

### Step G. 6-[(1-{[(2S)-8-Methyl-2,3-dihydro[1,4]dioxino[2,3-f]quinolin-2-yl]methyl}piperidin-3-yl)methyl]-2H-1,4-benzoxazin-3(4H)-one dihydrochloride salt

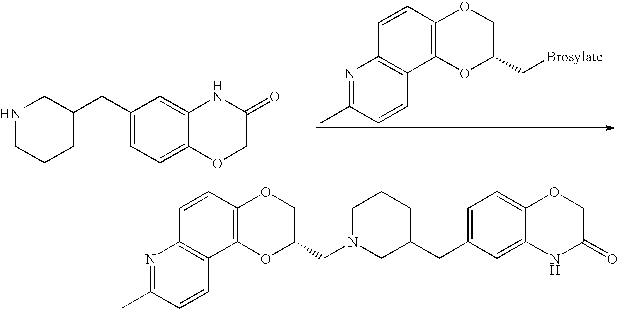

Using essentially the same procedure described for Step H of Example 14, the title compound was obtained as a mixture of diastereomers from 6-(piperidin-3-ylmethyl)-2H-1,4-benzoxazin-3(4H)-one hydrochloride salt of Step F in 50% yield as a yellow solid, m.p. 200-205° C.

MS [(+), m/z]: 460 [M+H]⁺

The diastereomers were separated by chiral HPLC and were converted to their di-hydrochloride salts:

Diastereomer 1: yellow solid, m.p. 200-205° C.

MS [(+), m/z]: 460 [M+H]+

[α]=−72.0° (1% EtOH).

Diastereomer 2: yellow solid; m.p. 200-205° C.

MS (+) 460 (M+H)+

[α]=−119.0° (1% EtOH)

### EXAMPLE 17

### 7-[(1-{[(2S)-8-Methyl-2,3-dihydro[1,4]dioxino[2,3-f]quinolin-2-yl]methyl}piperidin-4-yl)oxy]-3,4-dihydroquinolin-2(1 H)-one 2HCl

### Step A. 3-Chloro-N-(3-hydroxyphenyl)propanamide

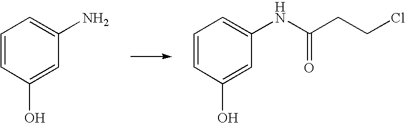

3-Chloropropanoyl chloride (9.6 g, 99 mmol) was added to a stirred mixture of 3-aminophenol (10 g, 92 mmol) and sodium bicarbonate (10 gram, 120 mmol) in a mixture of methanol (50 ml) and water (10 ml) dropwise at 0° C. over a period of 30 minutes. After the addition was complete the mixture was allowed to reach room temperature and was stirred for an additional 4 hours. It was then acidified with concentrated HCl and the product crystallized upon standing to afford the title compound (12.5 g, 67%).

¹HNMR(DMSO-d6): δ (ppm) 2.74 (t, 2H), 3.82 (t, 2H), 6.40 (d,1 H), 6.91 (d,1), 7.01 (d, 1H), 7.14 (s, 1H), 9.31 (s, 1H), 9.85 (s, 1H).

### Step B. 7-Hydroxy-3,4-dihydroquinolin-2(1H)-one

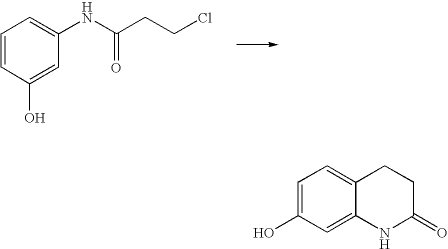

A mixture of 3-chloro-N-(3-hydroxyphenyl)propanamide of Step A (3.0 g, 15 mmol) and aluminum chloride (6.01 g, 45 mmol) was heated to 210° C. with stirring for 30 minutes until the gas evolution ceased. The mixture was then cooled to room temperature and quenched with water. The aqueous phase was extracted with ethyl acetate, dried over MgSO₄, filtered, and the solvent evaporated to leave a pink solid which was treated with cold ethyl acetate to afford the pure title compound (1.9 g, 76%), m.p. 218-220° C.

MS [(+) ESI, m/z]: 164 [M+H]⁺

### Step C. tert-Butyl 4-[(2-oxo-1,2,3,4-tetrahydroquinolin-7-yl)oxy]piperidine-1-carboxylate

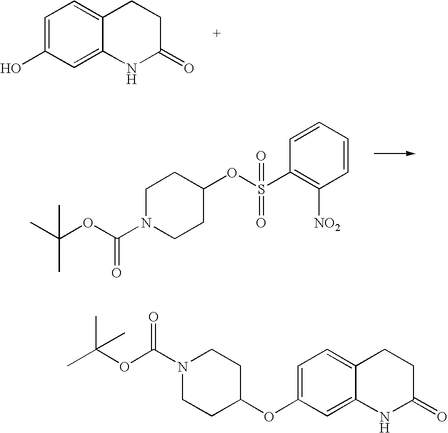

A solution of tert-butyl 4-{[(2-nitrophenyl)sulfonyl]oxy}piperidine-1-carboxylate of Example 3, step B (15.4 g, 40 mmol) in N,N-dimethylformamide (20 mL) was added to a stirred mixture of the 7-hydroxy-3,4-dihydroquinolin-2(1H)-one of step B (3.3 g, 20 mmol) and cesium carbonate (13.0 g, 40 mmol) in N,N-dimethylformamide (80 mL) dropwise at 50° C. over 60 minutes. After the addition was complete, the mixture was allowed to stir for an additional 6 hours before cooling to room temperature. The cooled mixture was partitioned between ethyl acetate and water. The organic phase was separated and dried over anhydrous magnesium sulfate. The product was purified by flash chromatography eluting with ethyl acetate/hexane to afford the title compound as a white solid (6 g, 87%), m.p. 158-160° C.

MS [(+) ESI, m/z]: 347 [M+H]⁺

### Step D. 7-(Piperidin-4-yloxy)-3,4-dihydroquinolin-2(1H)-one

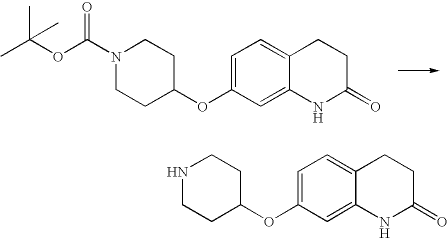

2M HCl in ether solution (34 mL, 68 mmol) was added to a solution of tert-butyl 4-[(2-oxo-1,2,3,4-tetrahydroquinolin-7-yl)oxy]piperidine-1-carboxylate of Step C (6.0 g, 17.3 mmol) in dichloromethane. The solution was stirred at room temperature overnight. The hydrochloride salt precipitated during the stirring was collected by filtration and washed with ether to afford the title compound as a white solid, m.p. 255-257° C. (4.6 g, 90% yield).

MS [(+) ESI, m/z]: 247.1 [M+H]⁺

### Step E. 7-[(1-{[(2S)-8-Methyl-2,3-dihydro[1,4]dioxino[2,3-f]quinolin-2-yl]methyl}piperidin-4-yl)oxy]-3,4-dihydroquinolin-2(1H)-one dihydrochloride salt

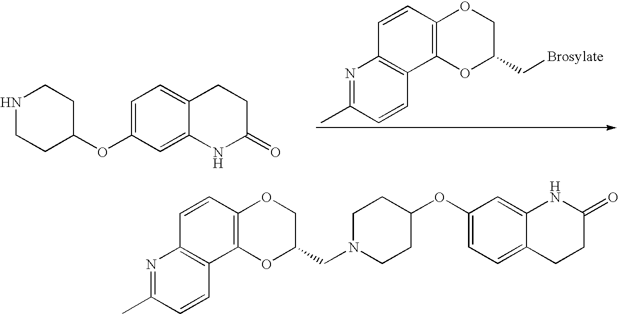

Using essentially the same procedure described for Step H of Example 14, the title compound was obtained from 7-(piperidin-4-yloxy)-3,4-dihydroquinolin-2(1H)-one of Step D in 52% yield, as a yellow solid.

MS [(+)ESI, m/z]: 460 [M+H]⁺

### EXAMPLE 18

### 6-(4-{[(8-methyl-2,3-dihydro[1,4]dioxino[2,3-f]quinolin-2-yl)methyl]amino}piperidin-1-yl)-2H-1,4-benzoxazin-3(4H)-one dihydrochloride salt

### Step A. Methyl (4-bromo-2-nitrophenoxy)acetate

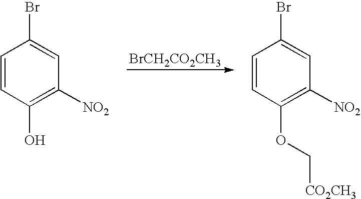

To a solution of 4-bromo-2-nitrophenol (6.54 g, 30 mmol) in acetone (90 mL) and N,N-dimethylformamide (10 mL) was added methyl bromoacetate (4.26 mL, 45 mmol), cesium carbonate (14.60 g, 45 mmol) and sodium iodide (2.25 g, 15 mmol) at room temperature. After stirring for 2 hours at 60° C., the solvents were removed under vacuum. The residue was diluted with water and extracted with ethyl acetate. The combined organic extracts were washed with water, brine, dried (MgSO₄) and concentrated to dryness to give the crude product which was triturated with hexane to give the title compound (8.20 g, 94%) as a solid.

MS [EI, m/z]: 289 [M]⁺

### Step B. 6-Bromo-2H-1,4-benzoxazin-3(4H)-one

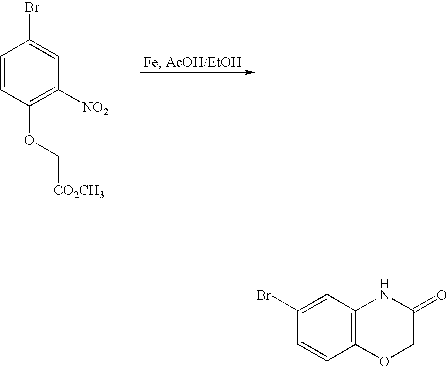

To a solution of methyl (4-bromo-2-nitrophenoxy)acetate from step A (8.1 g, 27.9 mmol) in acetic acid (75 mL) and ethanol (75 mL) was added iron powder (94.7 g, 83.8 mmol) at room temperature. After refluxing overnight, the reaction mixture was concentrated in vacuo. The residue was dissolved in ethyl acetate (300 mL), The mixture filtered through a pad of Celite and the cake washed with ethyl acetate. The filtrate was concentrated to dryness to give the title compound (5.9 g, 93%) as a solid. MS [(−)ESI,m/z]: 226 [M−H]⁻

### Step C. 6-(1,4-Dioxa-8-azaspiro[4.5]dec-8-yl)-2H-1,4-benzoxazin-3(4H)-one

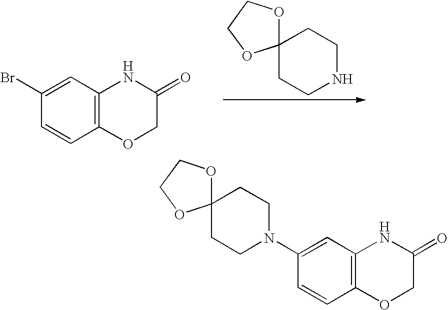

A mixture of 6-bromo-2H-1,4-benzoxazin-3(4H)-one of Step B (0.912 g, 4 mmol), 1,4-dioxa-8-azaspiro[4.5]decane (0.62 mL, 4.8 mmol), 2-(dicyclohexylphosphino)-2′-(N,N-dimethylamino)biphenyl (0.038 g), tris(dibenzylideneacetone)dipalladium (0) (0.037 g), and lithium bis(trimethylsilyl)amide (8.8 mL, 1.0 M in tetrahydrofuran, 8.8 mmol) in tetrahydrofuran (15 mL) was refluxed overnight. After cooling, the reaction was quenched with saturated aqueous ammonium chloride and diluted with ethyl acetate. The two layers were separated and the aqueous was extracted with ethyl acetate. The combined organic extracts were washed with water, brine, dried (MgSO₄) and concentrated. The crude material was purified by chromatography (silica gel, EtOAc/hexane 1:1) to afford the title compound (0.780 g, 67%) as a solid.

MS [(+)ESI, m/z]: 291 [M+H]⁺

### Step D. 6-(4-Oxopiperidin-1-yl)-2H-1,4-benzoxazin-3(4H)-one

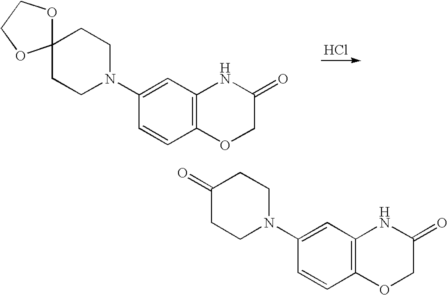

A mixture of 6-(1,4-dioxa-8-azaspiro[4.5]dec-8-yl)-2H-1,4-benzoxazin-3(4H)-one of Step C (0.770 g, 2.66 mmol) in 1N hydrochloric acid (15 mL) and tetrahydrofuran (15 mL) was heated at 80° C. overnight. After cooling, the reaction mixture was diluted with ethyl acetate and the two layers were separated. The aqueous layer was neutralized with solid sodium bicarbonate (to pH˜8) and extracted with ethyl acetate. The combined organic extracts were washed with water, brine, dried (MgSO₄) and concentrated to dryness to give the title compound (0.555 g, 85%) as a solid.

MS [(+)ESI, m/z]: 247 [M+H]⁺

### Step E. 6-(4-{[(8-methyl-2,3-dihydro[1,4]dioxino[2,3-f]quinolin-2-yl)methyl]amino}piperidin-1-yl)-2H-1,4-benzoxazin-3(4H)-one dihydrochloride salt

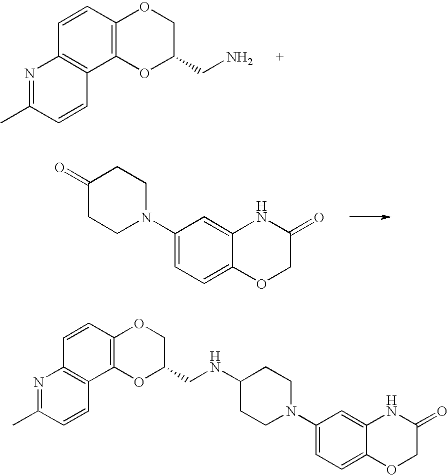

To a solution of 1-[(2S)(8-methyl-2,3-dihydro[1,4]dioxino[2,3-f]quinolin-2-yl]methanamine dihydrochloride salt (0.21 g, 0.4 mmol) in acetonitrile (10 mL) was added 6-(4-oxopiperidin-1-yl)-2H-1,4-benzoxazin-3(4H)-one of Step D (0.1 g, 4 mmol), acetic acid(0.23 mL, 4 mmol), N-ethyldiisopropylamine (0.15 mL, 0.84 mmol), and sodium triacetoxyborohydride (0.128 g, 0.6 mmol). The reaction mixture was stirred at room temperature overnight, basified with aqueous sodium carbonate, diluted with water and extracted with ethyl acetate. The organic layer was washed with water, and brine, dried over anhydrous magnesium sulfate and concentrated to dryness. The residue was purified by chromatography on silica gelusing as eluant: dichloromethane/MeOH (saturated with ammonia) 95:5, to provide the desired compound (0.04 g). The dihydrochloride salt was prepared as a yellow solid, m.p. 240° C. (dec) by treating a methanolic solution of the free base with 1N hydrochloric acid in ether.

MS [(−)ES, m/z]: 459.2 [M−H]⁻

### EXAMPLE 19

### 6-[1-(8-Methyl-2,3-dihydro-[1,4]dioxino[2,3-f]quinolin-2-ylmethyl)piperidin-4-yloxy]-4H-benzo[1,4]oxazin-3-one

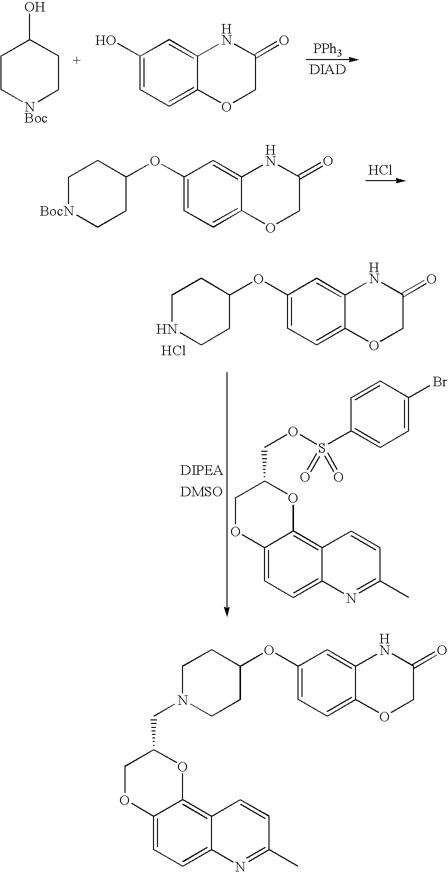

### Step A. 6-(Piperidin-4-yloxy)-4H-benzo[1,4]oxazin-3-one hydrochloride

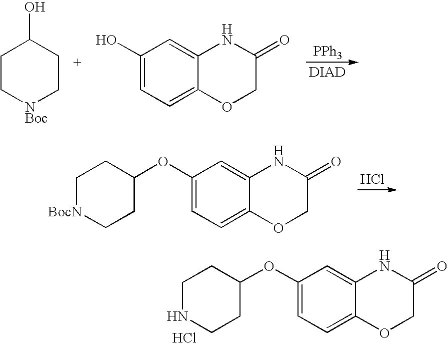

6-hydroxy-2H-benzo[b][1,4]oxazin-3(4H)-one (10.0 g, 60.55 mmol) was dissolved in THF (150 ml) under N₂. 1-BOC-4-hydroxypiperidine (14.62 g, 72.66 mmol) and triphenyl phosphine (20.96 g, 79.93) were added and the solution cooled to 0° C. DIAD (16.16 g, 79.93 mmol) was dissolved in 15 ml THF and added drop-wise over 1.5 h. The reaction was allowed to warm to room temperature over 3 h in the ice bath. After 4 h a solution of 5-6 N HCl in isopropanol (50 ml) was added and the reaction stirred 24 h. After this time the solution was filtered and the solids washed with 2×20 ml EtOAc. The filtrate was dried in a vacuum oven at 40° C. for 3 h giving 11.28 g (65%) of a white solid.

### Step B. 6-[1-(8-Methyl-2,3-dihydro-[1,4]dioxino[2,3-f]quinolin-2-ylmethyl)piperidin-4-yloxy]-4H-benzo[1,4]oxazin-3-one

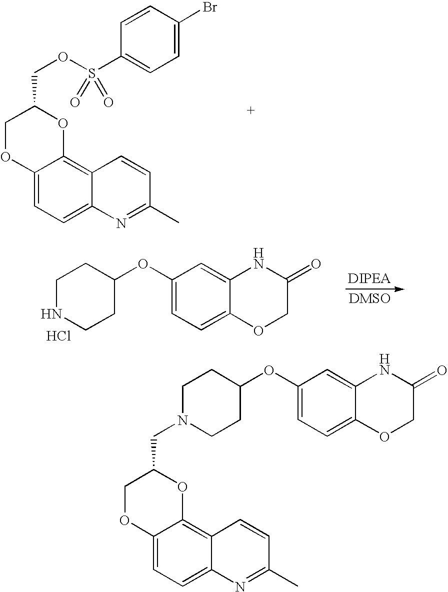

6-(Piperidin-4-yloxy)-4H-benzo[1,4]oxazin-3-one hydrochloride (3.16 g, 11.1 mmol) from Step A was dissolved in DMSO/H₂O (1:1 v/v, 6 ml). 10 N NaOH (1.1 ml) is then added resulting in a brown slurry. (8-methyl-2,3-dihydro-[1,4]dioxino[2,3-f]quinolin-2-yl)methyl 4-bromobenzenesulfonate in DMSO (4.4 ml) was added along with Hunig's base (1.58 g, 12.2 mmol). The reaction was heated to 70° C. for 6 h. After cooling to 40° C., water (30 ml) containing sodium bicarbonate (2.64 g) and potassium carbonate (0.7 g) was added. The water was decanted from the resulting gummy solid and ethanol (20 ml) was added. After stirring for 1 h, the resulting off-white solid was filtered and dried giving 6-[1-(8-methyl-2,3-dihydro-[1,4]dioxino[2,3-f]quinolin-2-ylmethyl)piperidin-4-yloxy]-2H-benzo[1,4]oxazin-3-one, 2.06 g (46%).

### EXAMPLE 20

### 7-[(1-{[(2S)-8-Methyl-2,3-dihydro[1,4]dioxino[2,3-f]quinolin-2yl]methyl}piperidin-4-yl)methyl]-3,4-dihydroquinolin-2(1H)-one

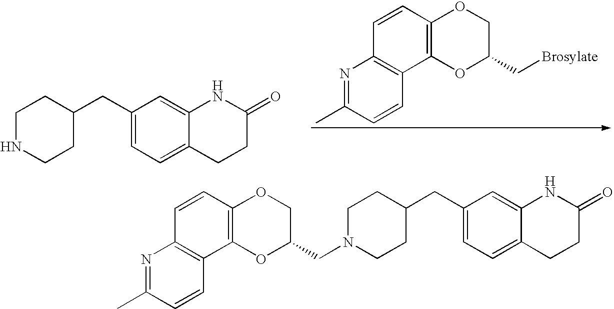

Using essentially the same procedure described for example 14 step H, the title compound was obtained as a yellow solid in 43% yield.

MS (+) 458 (M+H)⁺.

### EXAMPLE 21

### 8-Fluoro-6-[(1-{[(2S)-8-methyl-2,3-dihydro[1,4]dioxino[2,3-f]quinolin-2-yl]methyl}piperidin-4-yl)methyl]-2H-1,4-benzoxazin-3(4H)-one

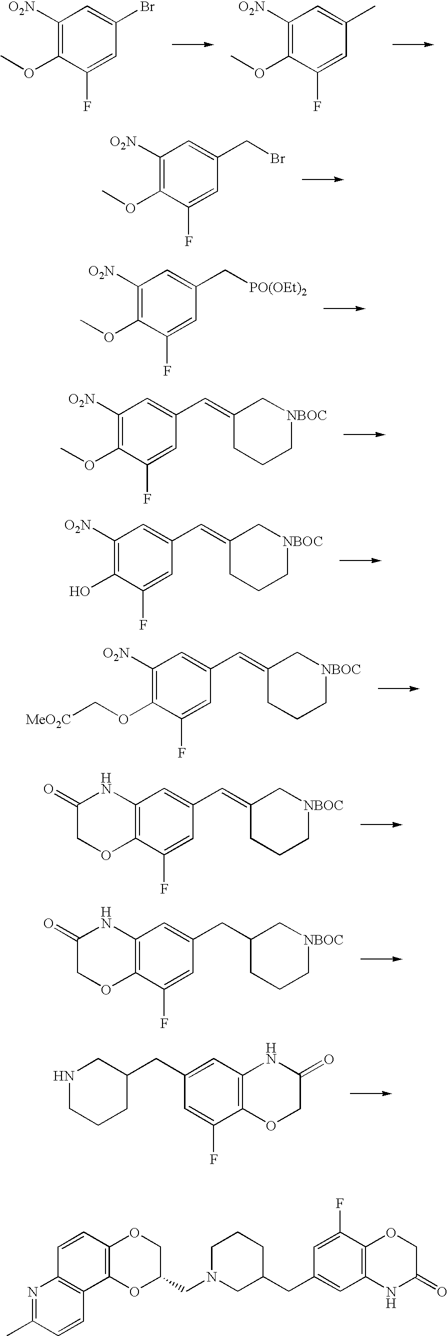

### Step A. 1-Fluoro-2-methoxy-5-methyl-3-nitrobenzene

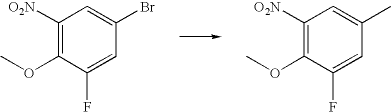

To a solution of 4-bromo-2-fluoro-6-nitrophenyl methyl ether (18 g, 72 mmol) in N,N-dimethyl formamide (30 ml) was added tetramethyl tin (6.26 ml, 38.16 mmol), palladium acetate (93 mg, 0.43 mmol), tris(4-methoxyphenyl)phosphine (0.53 g, 1.73 mmol), and N-ethyl-N-(1-methylethyl)propan-2-amine (36 mL, Step A (L37290-97) 223 mmol). The mixture was heated at 85° C. for 3 days, cooled to room temperature and filtered through Celite. The filtrate was diluted with water and extracted with ethyl acetate. The organic layer was concentrated to a residue, to which was added aqueous sodium carbonate solution. After stirring for a few minutes, the mixture was extracted with diethyl ether. The organic layer was washed with water then brine, dried (magnesium sulfate), filtered, and concentrated to afford 7.0 g (52%) of the title compound as an oil. MS (+) 186 (M+H)⁺.

### Step B. 4-(Bromomethyl)-2-fluoro-6-nitrophenyl methyl ether

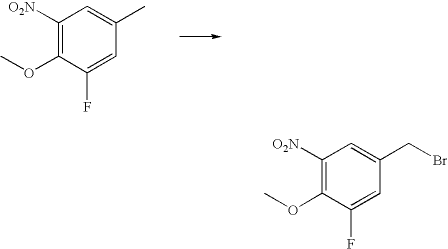

To a refluxing solution of 1-fluoro-2-methoxy-5-methyl-3-nitrobenzene (6.96 g, 37.62 mmol) in CCl₄ (200 mL) was added NBS (8 g, 45.1 mmol) and AIBN (0.185 g, 1.12 mmol) in 4 portions over 2 hours. After refluxing an additional 2 hours, the mixture was cooled to room temperature and filtered through a silica gel plug with 50% ethyl acetate in hexane. The filtrate was concentrated and triturated with 10% ethyl acetate in hexane. The resulting precipitate was collected by vacuum filtration, affording 4.0 g (40%) of the title compound as an off-white solid. MS (+) 265 (M+H)⁺.

### Step C. Diethyl (3-fluoro-4-methoxy-5-nitrobenzyl)phosphonate

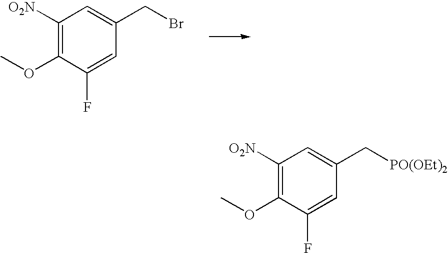

A solution of 4-(bromomethyl)-2-fluoro-6-nitrophenyl methyl ether (4.0 g, 15.15 mmol) in triethyl phosphate (4 ml) was heated at 120° C. for 2 hours, then was cooled to room temperature. Purification by silica gel chromatography afforded 4.1 g (84%) of the title compound as a yellow oil. MS (+) 321 (M+H)⁺.

### Step D. tert-Butyl -[(3-fluoro-4-methoxy-5-nitrophenyl)methylidene]piperidine-1-carboxylate

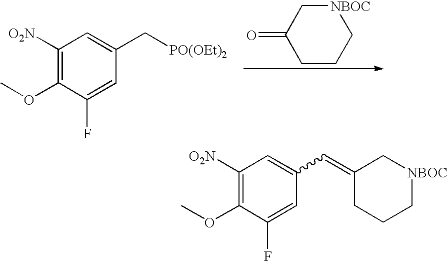

Using essentially the same procedure described for example 16 Step A, the title compound was obtained as an oil (2.3 g, 49%); MS (+) 367 (M+H)⁺.

### Step E. tert-Butyl -[(3-fluoro-4-hydroxy-5-nitrophenyl)methylidene]piperidine-1-carboxylate

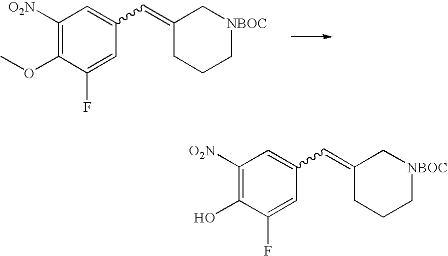

Using essentially the same procedure described for Example 22, Step G, the title compound was obtained in quantitative yield as a yellow foam. MS (+) 353 (M+H)⁺.

### Step F. tert-Butyl 3-{[5-fluoro-4-(2-methoxy-2-oxoethoxy)-3-nitrophenyl]methylidene}piperidine-1-carboxylate

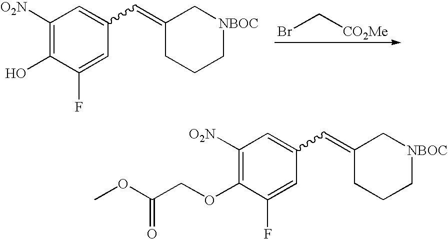

Using essentially the same procedure described for example 16 step C, the title compound was obtained as colorless oil (2.3 g, 85%); MS (+) 425 (M+H)⁺.

### Step G. tert-Butyl 3-[(8-fluoro-3-oxo-3,4-dihydro-2H-1,4-benzoxazin-6-yl)methylidene]piperidine-1-carboxylate

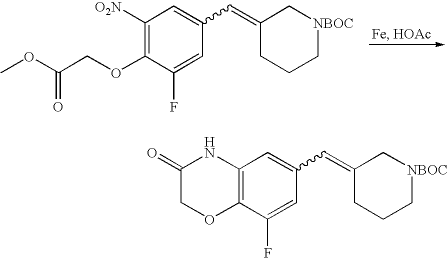

Using essentially the same procedure described for example 16 Step D, the title compound was obtained as a white foam (1.9 g, 97%); MS (+) 363 (M+H)⁺.

### Step H. tert-Butyl 3-[(8-fluoro-3-oxo-3,4-dihydro-2H-1,4-benzoxazin-6-yl)methyl]piperidine-1-carboxylate

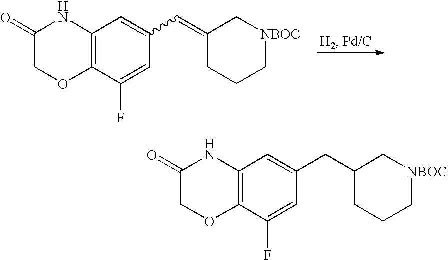

Using essentially the same procedure described for example 16 Step E, the title compound was obtained in quantitative yield as an off-white foam; MS (+) 365 (M+H)⁺.

### Step I. 8-Fluoro-6-(piperidin-3-ylmethyl)-2H-1,4-benzoxazin-3(4H)-one hydrochloride salt

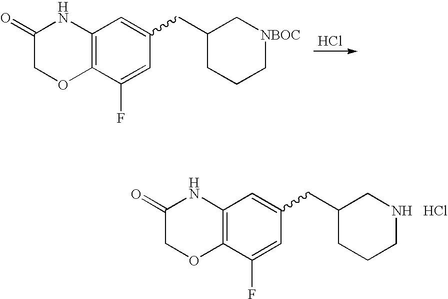

Using essentially the same procedure described for Example 14 step G, the title compound was obtained in quantitative yield as a white solid. MS (+) 265 (M+H)⁺.

### Step J. 8-Fluoro-6-[(1-{[(2S)-8-methyl-2,3-dihydro[1,4]dioxino[2,3-f]quinolin-2-yl]methyl}piperidin-4-yl)methyl]-2H-1,4-benzoxazin-3(4H)-one

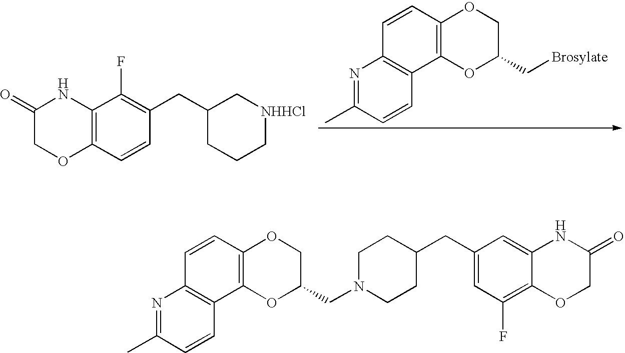

Using essentially the same procedure described for example 14 Step H, the title compound was obtained as a mixture of diastereomers in 54% yield as an off-white foam; MS (+) 478 (M+H)⁺.

The diastereomers were separated by chiral HPLC and were converted to their dihydrochloride salts:
21a. Diastereomer 1: yellow solid, m.p. 230° C. (dec); [α]=−3.0° (1% EtOH)
21b. Diastereomer 2: yellow solid, m.p. 230° C. (dec); [α]=−108.0° (1% EtOH)

### EXAMPLE 22

### 5-Fluoro-6-[(1-{[(2S)-8-methyl-2,3-dihydro[1,4]dioxino[2,3-f]quinolin-2-yl]methyl}piperidin-4-yl)methyl]-2H-1,4-benzoxazin-3(4H)-one

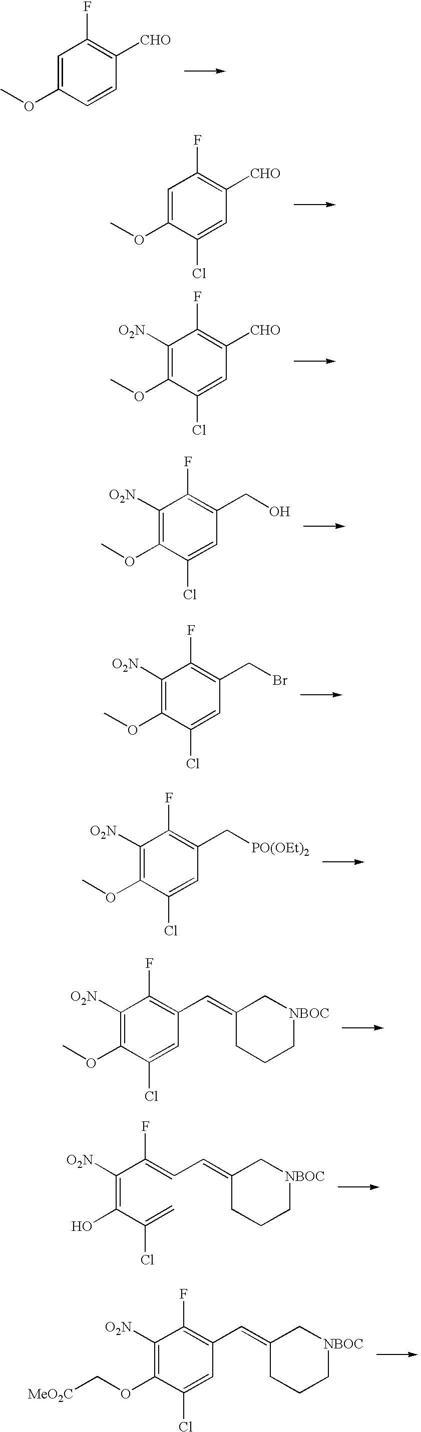

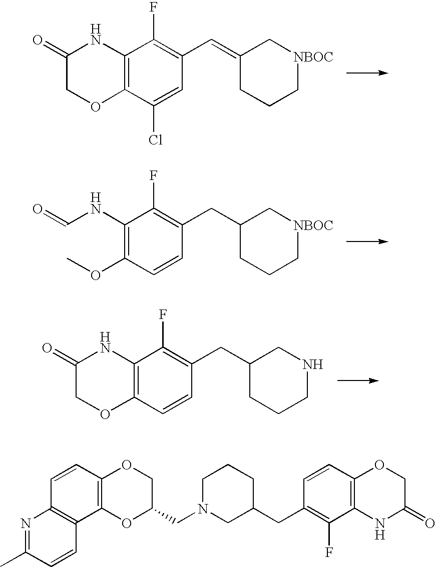

### Step A. 5-Chloro-2-fluoro-4-methoxybenzaldehyde

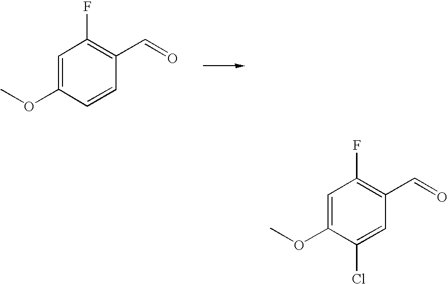

To a solution of 2-fluoro-4-methoxybenzaldehyde (3.08 g, 20 mmol) in acetic acid (6 ml) was added sulfuryl chloride (3.36 ml, 40 ml). After stirring at room temperature overnight, the reaction mixture was poured into ice water. The resultant white solid precipitate was collected by vacuum filtration, washed with water, and air dried to afford 3.8 g (quant.) of the title compound. MS (+) 189 (M+H)⁺.

### Step B. 5-Chloro-2-fluoro-4-methoxy-3-nitrobenzaldehyde

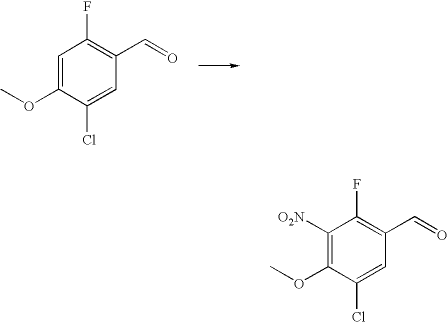

To a solution of conc. nitric acid (6.43 ml, 100 mmol) in conc. sulfuric acid (60 ml) was added 5-chloro-2-fluoro-4-methoxybenzaldehyde (18.86 g, 100 mmol) portionwise at −25° C. The mixture was stirred for 30 minutes while keeping the inner temperature bellow −15° C. The mixture was poured into ice water and extracted with dichloromethane. The organic layer was washed with aqueous sodium bicarbonate solution, water,and brine, then was dried (MgSO₄), filtered, and concentrated. Purification by chromatography on silica gel afforded 9.6 g (41%) of the title compound. MS (+) 234 (M+H)⁺.

### Step C. (5-Chloro-2-fluoro-4-methoxy-3-nitrophenyl)methanol

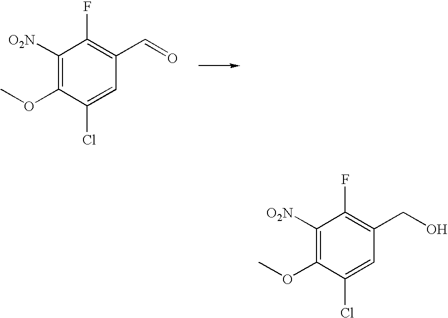

To a 0° C. solution of 5-chloro-2-fluoro-4-methoxy-3-nitrobenzaldehyde (9.6 g, 41.1 mmol) in methanol (100 ml) was added sodium borohydride (1.55 g, 41.1 mmol). The reaction was stirred at 0° C. for 30 minutes, then was acidified to pH=4 with 1N hydrochloric acid The methanol was removed in vacuo and the resulting aqueous suspension was extrated with ethyl acetate. The organic layer was washed with water, then brine, then was dried (magnesium sulfate), filtered and concentrated. Purification by chromatography on silica gel afforded 4.5 g (46%) of the title compound. MS (+) 235 (M+H)⁺.

### Step D. 1-(Bromomethyl)-5-chloro-2-fluoro-4-methoxy-3-nitrobenzene

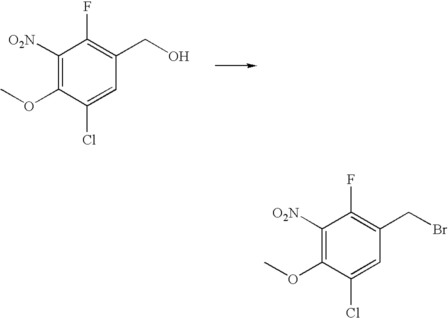

To a −5° C. solution of (5-chloro-2-fluoro-4-methoxy-3-nitrophenyl)methanol (3.8 g, 16.13 mmol) in tetrahydrofuran (70 mL) was added N-bromosuccinimide (3.45 g, 19.36 mmol ) and triphenylphosphine (5.07 g, 19.36 mmol). After stirring at 0° C. for 20 minutes, the reaction was filtered through a silica gel plug (3.5″×2″) with 20% ethyl acetate in hexane, Concetnration of the filtrate afforded a quantitative yield of the title compound. MS (+) 299 (M+H)^{+.}

### Step E. Diethyl (5-chloro-2-fluoro-4-methoxy-3-nitrobenzyl)phosphonate P6897-208

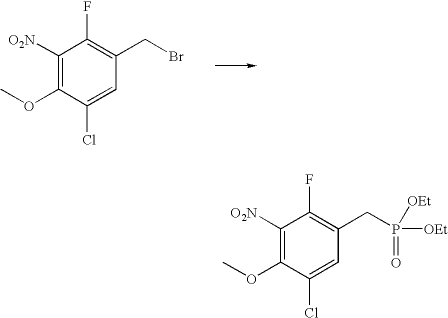

A mixture of 1-(bromomethyl)-5-chloro-2-fluoro-4-methoxy-3-nitrobenzene (4.02 g, 13.5 mmol) and triethylphosphite was heated at 120° C. for 2 hours, cooled to room temperature and chromatographed on silica gel to afford 4.39 g (91%) of the title compound as a colorless oil. MS (+) 356 (M+H)⁺.

### Step F. tert-Butyl 3-(5-chloro-2-fluoro-4-methoxy-3-nitrobenzyl)piperidine-1-carboxylate

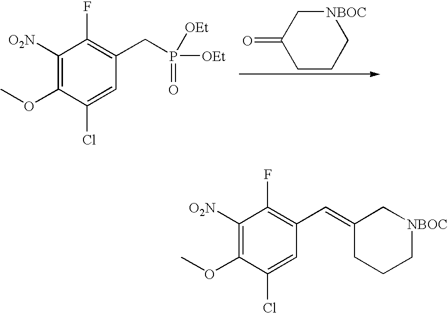

Using essentially the same procedure described for example 16 step A, the title compound was obtained as a colorless oil (1.8 g, 30%); MS (+) 401 (M+H)⁺.

### Step G. tert-Butyl 3-(5-chloro-2-fluoro-4-hydroxy-3-nitrobenzyl)piperidine-1-carboxylate

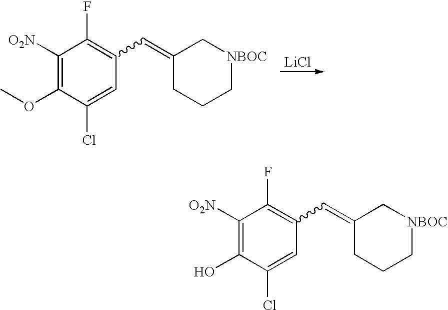

To a solution of tert-butyl 3-(5-chloro-2-fluoro-4-methoxy-3-nitrobenzyl)piperidine-1-carboxylate (1.8 g, 4.5 mmol) in N,N-dimethyl formamide (15 ml) was added lithium chloride (1.13 g, 27 mmol). The mixture was heated at reflux overnight, then cooled to room temperature. Water was added and the mixture was basified pH about 8-9 with saturated aqueous sodium carbonate solution and washed with diethyl ether. The aqueous layer was then acidified to pH=4, and extracted with diethyl ether. This organic layer was washed with water then brine, then was dried (magnesium sulfate), filtered, and concentrated to an orange foam, which was used in the next step without purification. MS (+) 387 (M+H)⁺.

### Step H. tert-Butyl (3E)-3-{[5-chloro-2-fluoro-4-(2-methoxy-2-oxoethoxy)-3-nitrophenyl]methylidene}piperidine-1-carboxylate

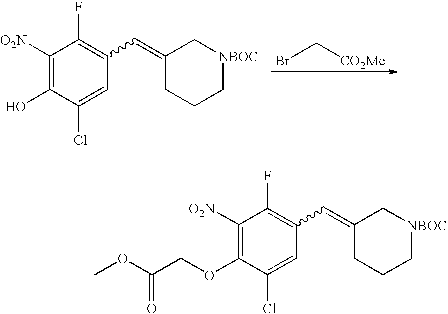

Using essentially the same procedure described for example 16 step C, the title compound was obtained as colorless oil (1.5 g, 78%); MS (+) 459 (M+H)⁺.

### Step I. tert-Butyl (3E)-3-[(8-chloro-5-fluoro-3-oxo-3,4-dihydro-2H-1,4-benzoxazin-6-yl)methylidene]piperidine-1-carboxylate

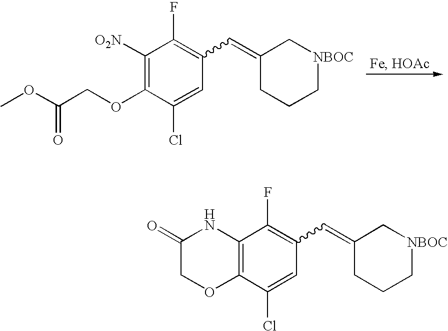

Using essentially the same procedure described for example 16 step D, the title compound was obtained in quantitative yield as a white foam. MS (+) 397 (M+H)⁺.

### Step J. tert-Butyl 3-[(5-fluoro-3-oxo-3,4-dihydro-2H-1,4-benzoxazin-6-yl)methyl]piperidine-1-carboxylate

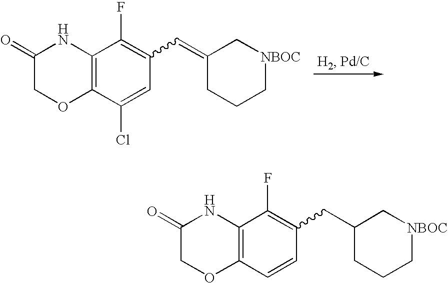

Using essentially the same procedure described for example 16 step E, the title compound was obtained as a white foam (1.1 g, 92%); MS (+) 401 (M+H)⁺.

### Step K. 5-Fuoro-6-(piperidin-3-ylmethyl)-2H-1,4-benzoxazin-3(4H)-one hydrochloride salt

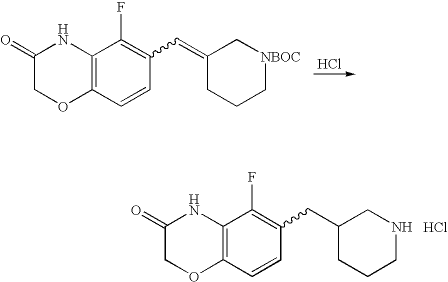

Using essentially the same procedure described for example 16 step F, the title compound was obtained in quantitative yield; MS (+) 265 (M+H)⁺.

### Step L. 5-Fluoro-6-[(1-{[(2S)-8-methyl-2,3-dihydro[1,4]dioxino[2,3-f]quinolin-2-yl]methyl}piperidin-4-yl)methyl]-2H-1,4-benzoxazin-3(4H)-one

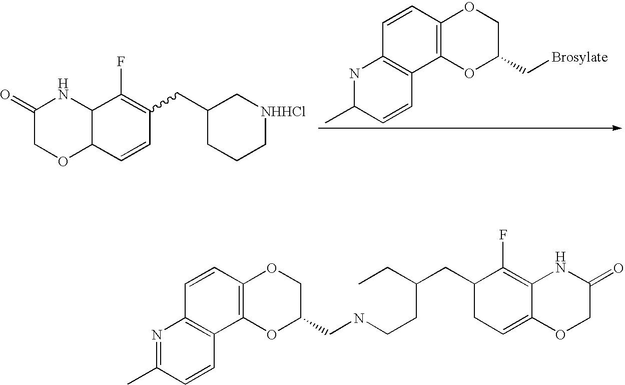

Using essentially the same procedure described for example 14 step H, the title compound was obtained as a mixture of diastereomers in 52% yield as an off-white foam; MS (+) 478 (M+H)⁺.

The diastereomers were separated by chiral HPLC and were converted to their dihydrochloride salts:
22a. Diastereomer 1: yellow solid, m.p. 231° C. (dec); [α]=−57.0° (1% EtOH)
22b. Diastereomer 2: yellow solid, m.p. 231° C. (dec); [α]=−79.0° (1% EtOH)

### EXAMPLE 23

### 7-[(8-{[(2S)-8-Methyl-2,3-dihydro[1,4]dioxino[2,3-f]quinolin-2-yl]methyl}-8-azabicyclo[3.2.1]oct-3-yl)methyl]-3,4-dihydroquinolin-2(1H)-one

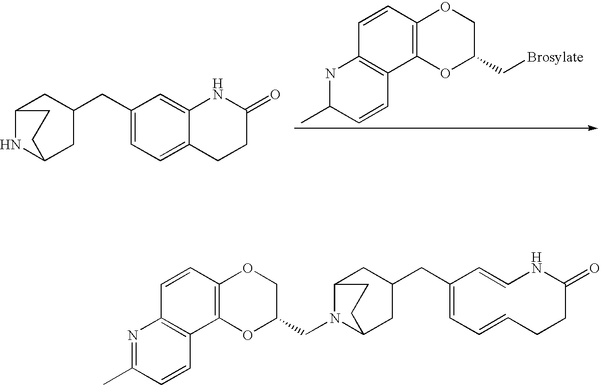

Using essentially the same procedure described for example 14 step H, the title compound was obtained as a yellow solid in 41% yield; MS (+) 484 (M+H)⁺.

### EXAMPLE 24

### 7-[(1-{[(2S)-8-Methyl-2,3-dihydro[1,4]dioxino[2,3-f]quinolin-2-yl]methyl}piperidin-3-yl)methyl]-3,4-dihydroquinolin-2(1H)-one

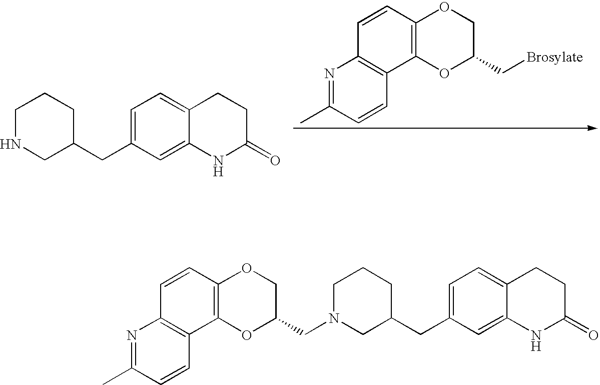

Using essentially the same procedure described for example 14 step H, the title compound was obtained as a yellow solid in 51% yield; MS (+) 458 (M+H)⁺.

### EXAMPLE 25

### 6-{[(3R)-1-{[(2S)-8-Methyl-2,3-dihydro[1,4]dioxino[2,3-f]quinolin-2-yl]methyl}piperidin-3-yl]oxy}-2H-1,4-benzoxazin-3(4H)-one

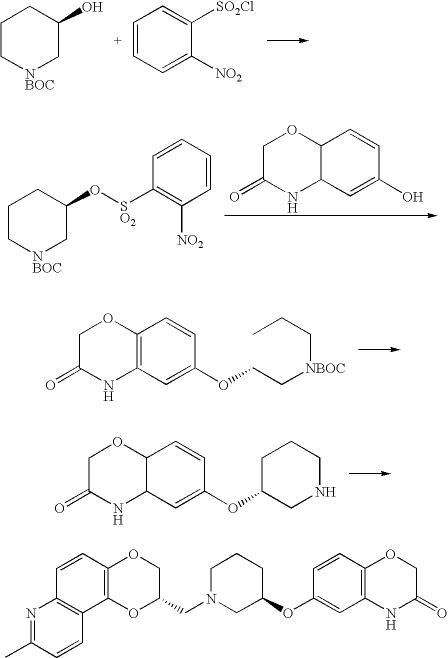

### Step A tert-Butyl (3R)-3-{[(2-nitrophenyl)sulfonyl]oxy}piperidine-1-carboxylate

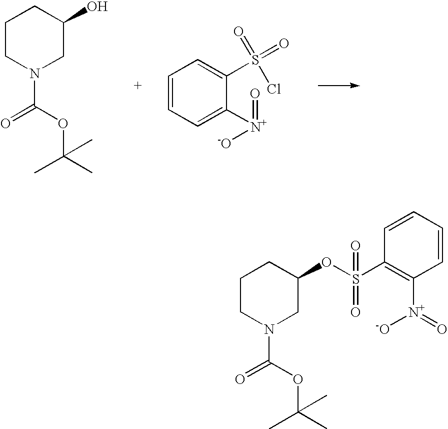

Using essentially the same procedure described for example 3 Step B, the title compound was obtained as a yellow oil (60%); MS (+) 387 (M+H)⁺.

### Step B. tert-Butyl (3R)-3-[(3-oxo-3,4-dihydro-2H-1,4-benzoxazin-6-yl)oxy]piperidine-1-carboxylate

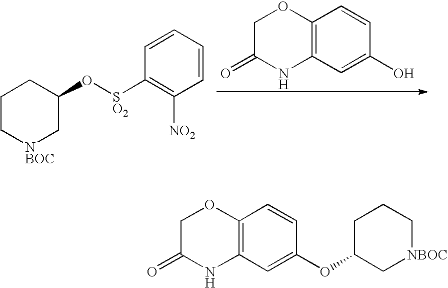

Using essentially the same procedure described for example 3 Step C, the title compound was obtained as a colorless oil (36%); MS (+) 349 (M+H)⁺.

### Step C. 6-[(3R)-Piperidin-3-yloxy]-2H-1,4-benzoxazin-3(4H)-one hydrochloride salt

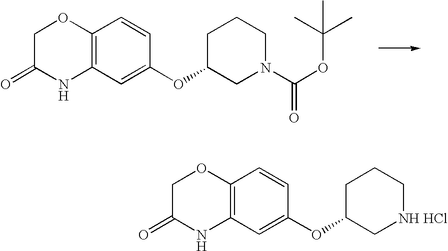

Using essentially the same procedure described for example 3 Step D, the title compound was obtained as a white solid; MS (+) 249 (M+H)⁺.

### Step D. 6-{[(3R)-1-{[(2S)-8-Methyl-2,3-dihydro[1,4]dioxino[2,3-f]quinolin-2-yl]methyl}piperidin-3-yl]oxy}-2H-1,4-benzoxazin-3(4H)-one

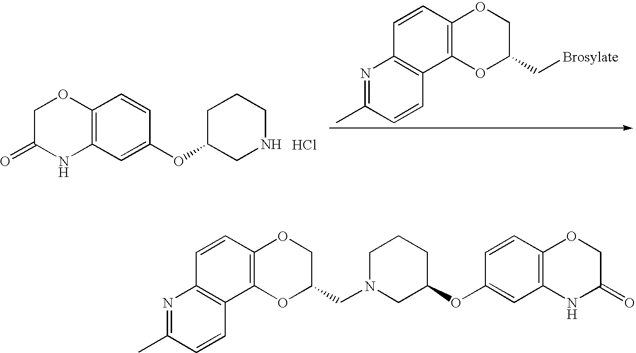

Using essentially the same procedure described for example 1 Step D, the title compound was obtained as a white solid; HRMS: calcd for [C₂₆H₂₇N₃O₃+H⁺] 462.20235; obs'd (ESI, M+H⁺) 462.2029; [α]=−101.0° (c=1% EtOH)

### EXAMPLE 26

### 6-{[(3S)-1-{[(2S)-8-Methyl-2,3-dihydro[1,4]dioxino[2,3-f]quinolin-2-yl]methyl}piperidin-3-yl]oxy}-2H-1,4-benzoxazin-3(4H)-one

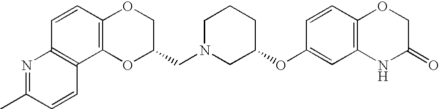

Using essentially the same procedure described for Example 25, the title compound was obtained as a yellow solid; HRMS: calcd for [C₂₆H₂₇N₃O₃+H⁺] 462.20235; obs'd (ESI, M+H⁺) 462.2023; [α]D25=−36.0° (c=1% EtOH)

When ranges are used herein for physical properties, such as molecular weight, or chemical properties, such as chemical formulae, all combinations and subcombinations of ranges specific embodiments therein are intended to be included.

The disclosures of each patent, patent application, and publication cited or described in this document are hereby incorporated herein by reference, in their entirety.

Those skilled in the art will appreciate that numerous changes and modifications can be made to the disclosed embodiments of the invention and that such changes and modifications can be made without departing from the spirit of the invention. It is, therefore, intended that the appended claims cover all such equivalent variations as fall within the true spirit and scope of the invention.

What is claimed is:

1. A compound having the Formula I: 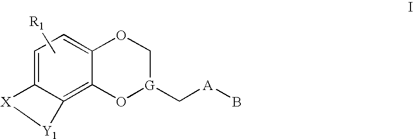
or pharmaceutically-acceptable salt thereof;
wherein:
G is a carbon atom having a hydrogen atom attached thereto, wherein the carbon atom can have either the R or S configuration;
R₁ is hydrogen, hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;
the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, hydroxy, C₁₋₁₂ alkoxy, halo, C₁₋₆ alkyl, amino, or mono- or di-alkylamino in which each alkyl group has 1 to 6 carbon atoms;
R₄ is hydrogen or C₁₋₆ alkyl;
A is -Q₁-Q₂-;
Q₁ is —N(R₈)—, 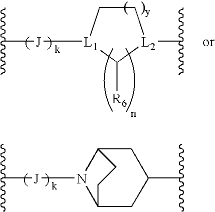
wherein:
J is NH;
k is 0 or 1;
L₁ is N, or CH;
L₂is N or CH;
R₆ is hydrogen or C₁₋₁₂ alkyl;
R₈ is hydrogen, C₁₋₁₂ alkyl C₄₋₂₀ alkylcycloalkyl, or C₃₋₈ cycloalkyl;
n is 1, 2 or 3;
y is 1 or 2, provided that when y is 2, n is 1;
provided that when k is 1, then L₁ is CH;
Q₂ has the Formula III or IV: 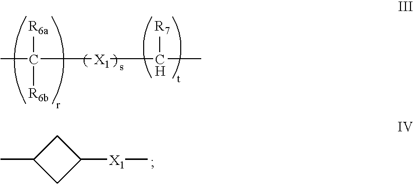
wherein:
each R₆ₐ and each R_{6b} is independently hydrogen or C₁₋₁₂ alkyl;
each R₇ is independently hydrogen or C₁₋₁₂ alkyl;
X₁ is O, NH or —(CH₂)ᵥ— where v is 1, 2, 3 or 4;
r is 0, 1, 2, 3 or 4;
s is 0 or 1;
t is 0, 1, 2 or 3;
provided that when when Q₂ has the Formula IV where X₁ is O or (CH₂)ᵥ then Q₁ is N(R₈);
B has the Formula: 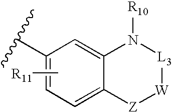
wherein:
R₁₀ is hydrogen, C₁₋₁₂ alkyl or C₄₋₂₀ alkylcycloalkyl, (CH₂)x-cycloalkyl;
L₃ is —C(═W′)— or CH₂;
W′ is O or S;
W is O, CH₂ or 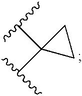
Z is O, CH₂ S or SO₂;
R₁₁ is hydrogen, C₁₋₁₂alkyl, cyano or halogen;
provided that Z and W are not both O; and
provided that W is not O when Z is S or SO₂.

2. A compound, or pharmaceutically acceptable salt thereof, -or pharmaceutically acceptable salt thereof, according to claim 1, wherein:
—X—Y₁— is —N═C(R₂)—C(R₄)═CH—;
R₁ is hydrogen;
—X—Y₁— is —N═C(R₂)—C(R₄)═CH—;
R₂ is hydrogen or C₁₋₄ alkyl;
R₄ is hydrogen or C₁₋₄ alkyl;
A is Q₁-Q_{2,} where
Q₁ is —N(R₈)—, 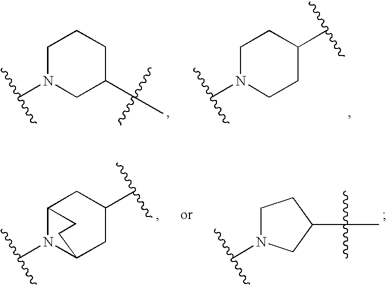
where R₈ is hydrogen or C₁₋₄ alkyl;
Q₂ is —O—, or —(CH₂)ᵥ— where v is 1 or 2;
B is 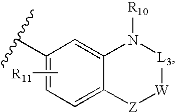
where
R₁₀ is hydrogen or C₁₋₄ alkyl;
L₃ is C=O;
W is CH₂;
Z is C or O; and
R₁₁ is hydrogen or halogen.

3. A compound, or pharmaceutically acceptable salt thereof, or pharmaceutically acceptable salt thereof, according to claim 2, wherein Q1 is N(R8).

4. A compound, or pharmaceutically acceptable salt thereof, of claim 2, wherein
Q₁ is 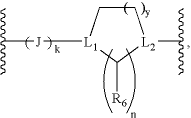
where J is NH, k is 1.

5. A compound or pharmaceutically acceptable salt thereof, of claim 4, wherein L₁ is CH, y is 1, L₂ is N, R₆ is H, and n is 2.

6. A compound or pharmaceutically acceptable salt thereof, of claim 5, wherein r, s, and t of Q₂ are each 0.

7. A compound, or pharmaceutically acceptable salt thereof, or pharmaceutically acceptable salt thereof, of claim 2, wherein
Q1 is

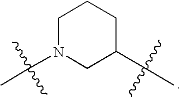

8. A compound, or pharmaceutically acceptable salt thereof, or pharmaceutically acceptable salt thereof, according to claim 7, wherein Q₂ is —O—.

9. A compound, or pharmaceutically acceptable salt thereof, or pharmaceutically acceptable salt thereof, according to claim 7, wherein Q₂ is —CH2—.

10. A compound, or pharmaceutically acceptable salt thereof, or pharmaceutically acceptable salt thereof, according to claim 2, wherein
Q1 is

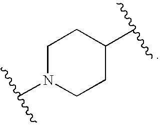

11. A compound, or pharmaceutically acceptable salt thereof, or pharmaceutically acceptable salt thereof, according to claim 10, wherein Q2 is —O—.

12. A compound, or pharmaceutically acceptable salt thereof, according to claim 10, wherein Q2 is —(CH2)ᵥ— where v is 1 or 2.

13. A compound, or pharmaceutically acceptable salt thereof, according to claim 2, wherein
Q1 is

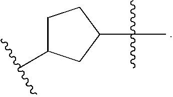

14. A compound, or pharmaceutically acceptable salt thereof, of claim 13, wherein Q2 is —O—.

15. A compound, or pharmaceutically acceptable salt thereof, of claim 2, wherein
Q1 is

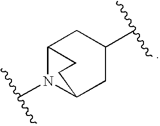

16. A compound, or pharmaceutically acceptable salt thereof, of claim 15, wherein Q2 is —CH2—.

17. The compound of claim 1, wherein Z is O; W is CH₂; and L₃ is —C(═W′)— wherein W′ is O.

18. The compound of claim 1, wherein Z is O; W is CH₂; L₃ is —C(═W′)— wherein W′ is O; and R₁₀ is hydrogen or C₁₋₆ alkyl.

19. The compound of claim 1, wherein the group X—Y₁ is —N═C(R₂)—C(R₃)═N—.

20. The compound of claim 1, wherein the group X—Y₁ is —N═C(R₂)—C(R₄)═CH—.

21. The compound of claim 1, wherein the group X—Y₁ is —N═C(R₂)—N═CH—.

22. The compound of claim 1, wherein the group X—Y₁ is —N═C(R₂)—O—.

23. The compound of claim 20, wherein Z is O; W is CH₂; L₃ is —C(═W′)— wherein W′ is O.

24. The compound of claim 23, wherein R₁₀ is hydrogen or C₁₋₆ alkyl; R₁₁ is hydrogen; R₂ is methyl; and R₄ is hydrogen.

25. The compound of claim 23, wherein Q₁ has the Formula II; and Q₂ has the Formula III.

26. The compound of claim 23, wherein Q₁ is —N(R₈)—.

27. The compound of claim 26, wherein Q₂ has the Formula IV, wherein X₁ is O or CH₂.

28. The compound of claim 26, wherein Q₂ has the Formula III, wherein r is 2, 3, or 4; s is 1; X₁ is NH; and t is 1.

29. The compound of claim 26, wherein Q₂ has the Formula III, wherein r is 3; s is 1; X₁ is O; and t is 0.

30. The compound of claim 26, wherein Q₂ has the Formula III, wherein r is 2; s is 1; X₁ is O; and t is 0.

31. The compound of claim 26, wherein Q₂ has the Formula III, wherein r is 3; s is 0; and t is 0.

32. The compound of claim 26, wherein Q₂ has the Formula III, wherein r is 2, 3 or 4; s is 0; and t is 0.

33. The compound of claim 26, wherein Q₂ has the Formula III, wherein r is 3 or 4; s is 1; X₁ is O; and t is 0.

34. The compound of claim 25, wherein Q₁ has the Formula:

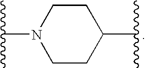

35. The compound of claim 34, wherein Q₂ is O, CH₂ or CH₂CH₂.

36. The compound of claim 35, wherein R₁₀ is hydrogen or C₁₋₆ alkyl.

37. The compound of claim 36, wherein R₂ is methyl, and R₄ is hydrogen.

38. The compound of claim 25, wherein Q₁ has the formula:

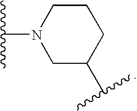

39. The compound of claim 38, wherein Q₂ is O, CH₂ or CH₂CH₂.

40. The compound of claim 25, wherein Q₁ has the formula:

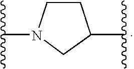

41. The compound of claim 40, wherein Q₂ is O, CH₂ or CH₂CH₂.

42 The compound of claim 1, wherein the carbon atom of said G has the S configuration.

43. The compound of claim 42, wherein said compound is substantially free of the R enantiomer of said compound.

44. The compound of claim 1, having one of the following Formulas: 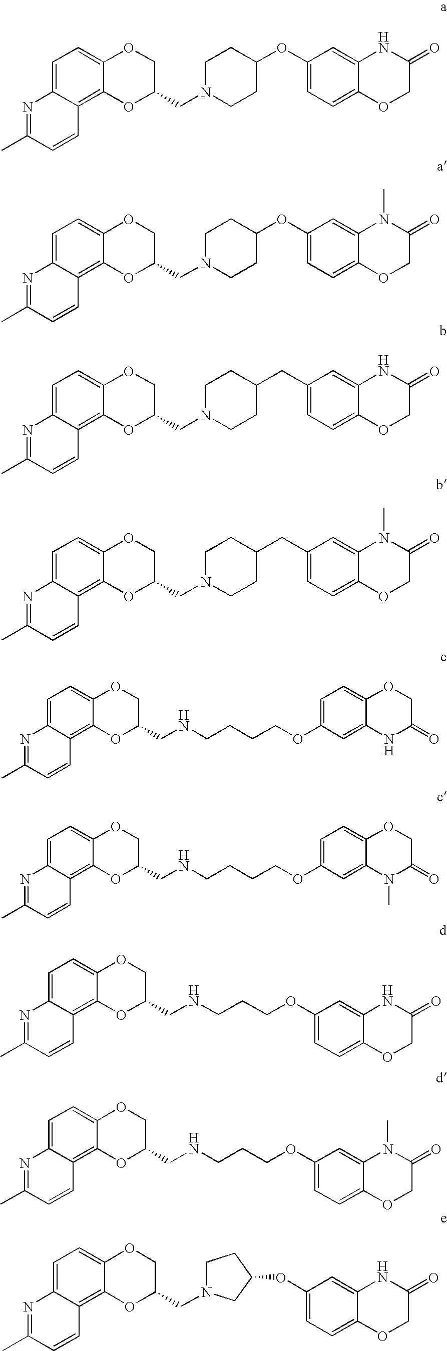 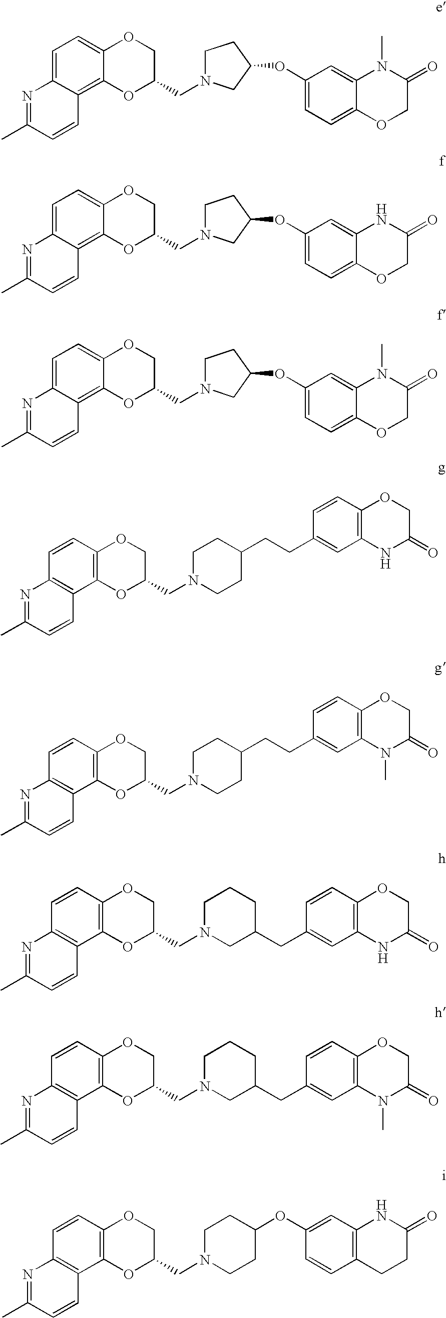 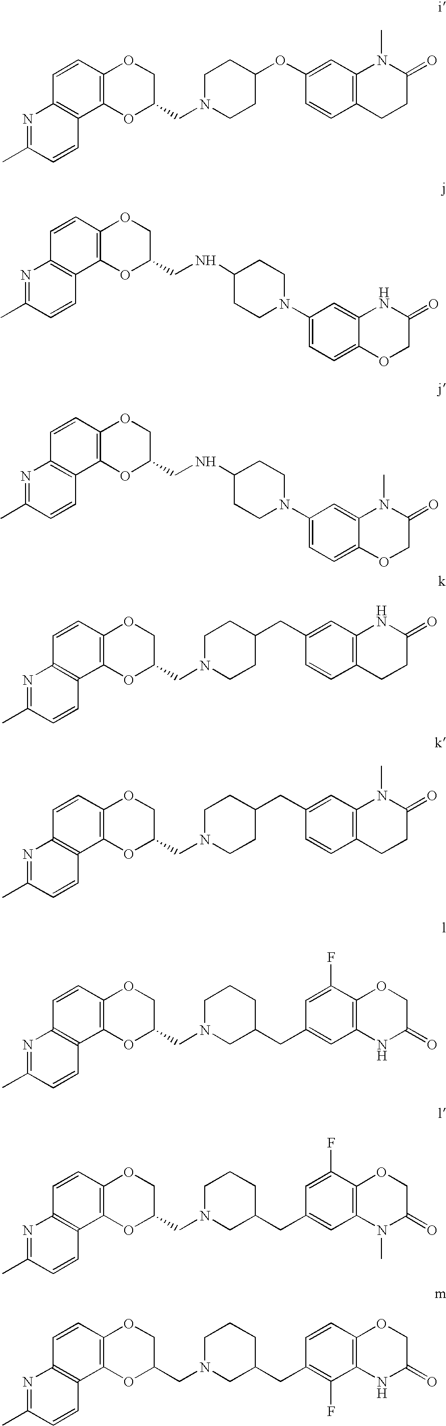 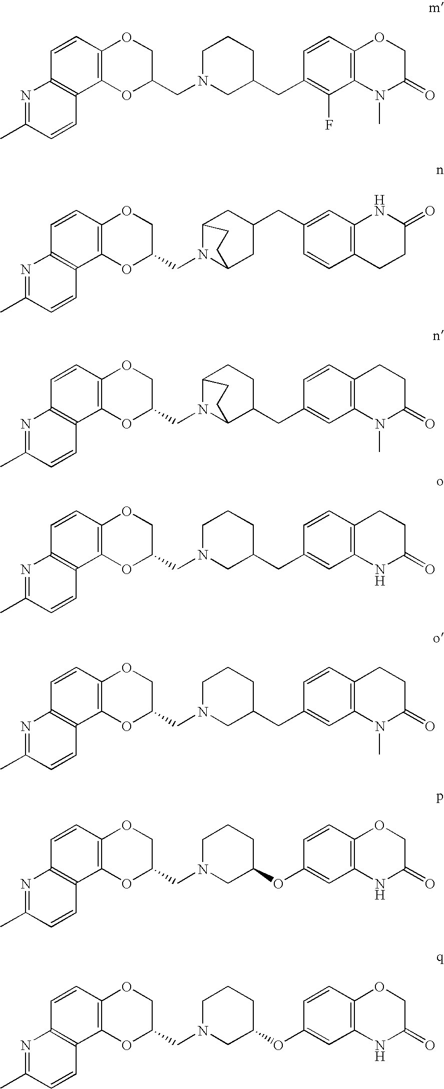
or a pharmaceuticall acceptable salt thereof.

45. A method of treating a subject suffering from a condition selected from depression, anxiety, panic disorder, post-traumatic stress disorder, premenstrual dysphoric disorder, attention deficit disorder, obsessive compulsive disorder, social anxiety disorder, generalized anxiety disorder, obesity, eating disorders, vasomotor flushing, cocaine and alcohol addiction, and sexual dysfunction, comprising the step of:
administering to said subject suffering from said condition, a therapeutically effective amount of a compound according to claim 1, or a pharmaceutically acceptable salt thereof.

46. A pharmaceutical composition, comprising:
an effective amount of a compound according to claim 1, or a pharmaceutically acceptable salt thereof; and
a pharmaceutically acceptable carrier or excipient.

47. A method comprising the step of:
a) reacting a compound of Formula S1: 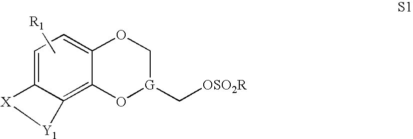
wherein:
G is a carbon atom having a hydrogen atom attached thereto, wherein the carbon atom can have either the R or S configuration;
R is a an appropriately substituted phenyl moiety;
R₁ is hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;
the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, halo, C₁₋₆ alkyl, amino, or mono- or di-alkylamino in which each alkyl group has 1 to 6 carbon atoms;
R₄ is hydrogen or C₁₋₆ alkyl;
with a compound of Formula S2: 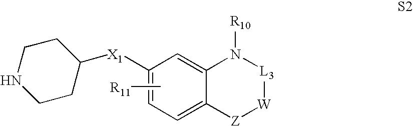
wherein:
R₁₀ is hydrogen, C₁₋₁₂ alkyl or (CH₂)ₓcycloalkyl), where x is 4-20;
L₃ is —C(═W′)— or CH₂;
W′ is O or S;
W is O CH₂ or 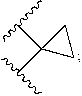
Z is O CH₂ S, or SO₂;
R₁₁ is hydrogen, C₁₋₁₂alkyl, cyano or halogen;
provided that Z and W are not both O; and
provided that W is not O when Z is S or SO₂;
for a time and under conditions effective to form a compound of Formula S3:

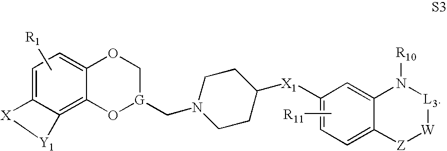

48. A method comprising the step of:
a) reacting a compound of Formula S10: 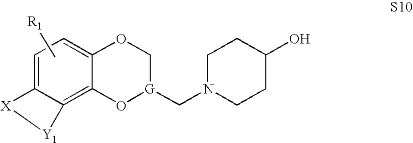
wherein:
G is a carbon atom having a hydrogen atom attached thereto, wherein the carbon atom can have either the R or S configuration;
R₁ is hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;
the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, halo, C₁₋₆ alkyl, amino, or mono- or di-alkylamino in which each alkyl group has 1 to 6 carbon atoms;
R₄ is hydrogen or C₁₋₆ alkyl;
with a compound of Formula S11: 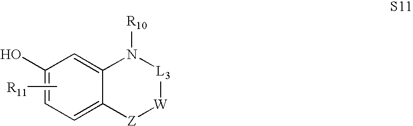
wherein:
R₁₀ is hydrogen, C₁₋₁₂ alkyl or C₄₋₂₀ alkylcycloalkyl;
L₃ is —C(═W′)— or CH₂;
W′ is O or S;
W is O, CH₂ or 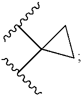
Z is O, CH₂ S or SO₂;
R₁₁ is hydrogen, C₁₋₁₂alkyl, cyano or halogen;
provided that Z and W are not both O; and
provided that W is not O when Z is S or SO₂;
for a time and under conditions effective to form a compound of Formula S12:

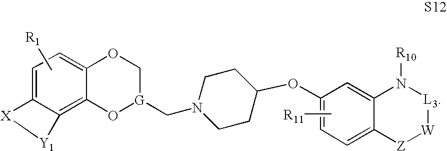

49. A method of preparing a compound of formula I 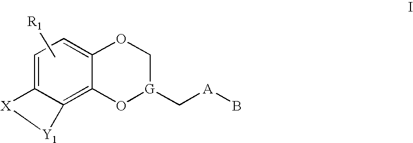
or pharmaceutically-acceptable salt thereof;
wherein:
G is a carbon atom having a hydrogen atom attached thereto, wherein the carbon atom can have either the R or S configuration;
R₁ is hydroxy, halo, cyano, carboxamido, carboalkoxy of 2 to 6 carbon atoms, trifluoromethyl, alkyl of 1 to 6 carbon atoms, alkanoyloxy of 2 to 6 carbon atoms, amino, mono- or di-alkylamino in which each alkyl group has 1 to 6 carbon atoms, alkanamido of 2 to 6 carbon atoms, or alkanesulfonamido of 1 to 6 carbon atoms;
the group X—Y₁ is —N═C(R₂)—C(R₃)═N—, —N═C(R₂)—C(R₄)═CH—, —N═C(R₂)—N═CH—, or —N═C(R₂)—O—;
R₂ and R₃ are, independently, hydrogen, hydroxy, C₁₋₁₂ alkoxy, halo, C₁₋₆ alkyl, amino, or mono- or di-alkylamino in which each alkyl group has 1 to 6 carbon atoms;
R₄ is hydrogen or C₁₋₆ alkyl;
A is -Q₁-Q₂-;
Q₁ is a group of Formula II: 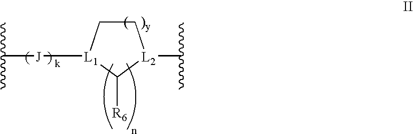
wherein:
J is NH;
k is 1;
L₁ is CH;
L₂ is N;
R₆ is hydrogen;
R₈ is hydrogen, C₁₋₁₂ alkyl C₄₋₂₀ alkylcycloalkyl, or C₃₋₈ cycloalkyl;
n is 2;
y is 1;
Q₂ has the Formula III 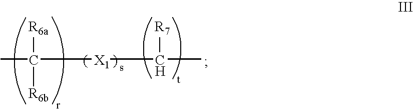
wherein:
each R₆ₐ and each R_{6b} is independently hydrogen or C₁₋₁₂ alkyl;
each R₇ is independently hydrogen or C₁₋₁₂ alkyl;
X₁ is O, NH or —(CH₂)ᵥ— where v is 1, 2, 3 or 4;
r is 0;
s is 0;
t is 0;
provided that when when Q₂ has the Formula IV where X₁ is O or (CH₂)ᵥ then Q₁ is N(R₈);
B has the Formula: 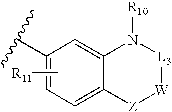
wherein:
R₁₀ is hydrogen, C₁₋₁₂ alkyl or C₄₋₂₀ alkylcycloalkyl, (CH₂)ₓ-cycloalkyl;
L₃ is —C(═W′)— or CH₂;
W′ is O or S;
W is O, CH₂ or 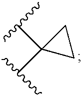
Z is O, CH₂ S or SO₂;
R₁₁ is hydrogen, C₁₋₁₂alkyl, cyano or halogen;
provided that Z and W are not both O; and
provided that W is not O when Z is S or SO₂; comprising the steps of: 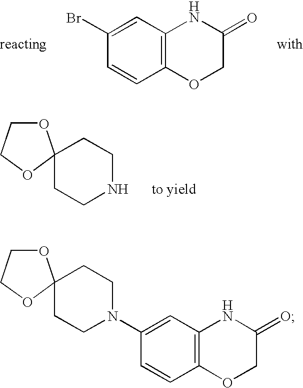
treating the resultant product with acid to yield 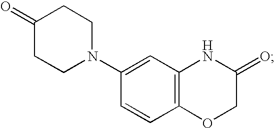
performing reductive amination with a compound of formula 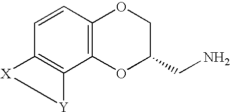
to produce a compound of formula I.
